SEQUENCE LISTING (74 sequences)

SEQ ID NO: 1 - protein, 545 aa, Artificial Sequence
  Synthetic polymer.
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 2 - protein, 144 aa, Artificial Sequence
  Synthetic polymer.
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 3 - protein, 254 aa, Artificial Sequence
  Synthetic polymer.
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 4 - protein, 205 aa, Artificial Sequence
  Synthetic polymer.
ACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 5 - protein, 234 aa, Artificial Sequence
  Synthetic polymer.
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 6 - protein, 172 aa, Artificial Sequence
  Synthetic polymer.
QRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 7 - protein, 233 aa, Artificial Sequence
  Synthetic polymer.
MDNQGVIYSDLNLPPNPKRQQRKPKGNKNSILATEQEITYAELNLQKASQDFQGNDKTYHCKDLPSAPEKLIVGILGIICLILMASVVTIVVIPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 8 - protein, 140 aa, Artificial Sequence
  Synthetic polymer.
PSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 9 - protein, 583 aa, Artificial Sequence
  Synthetic polymer.
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 10 - protein, 550 aa, Artificial Sequence
  Synthetic polymer.
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 11 - protein, 518 aa, Artificial Sequence
  Synthetic polymer.
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQSKYGPPCPPCPAPEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGKIEGRMDPSTLIQRHNNSSLNTRTQKARHCGHCPEEWITYSNSCYYIGKERRTWEESLLACTSKNSSLLSIDNEEEMKFLSIISPSSWIGVFRNSSHHPWVTMNGLAFKHEIKDSDNAELNCAVLQVNRLKSAQCGSSIIYHCKHKL

SEQ ID NO: 12-24 - skipped (13 entries)

SEQ ID NO: 25 - protein, 217 aa, Artificial Sequence
  Synthetic polymer.
APEFLGGPSVFLFPPKPKDTLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVMHEALHNHYTQKSLSLSLGK

SEQ ID NO: 26 - protein, 217 aa, Artificial Sequence
  Synthetic polymer.
APEFLGGPSVFLFPPKPKDQLMISRTPEVTCVVVDVSQEDPEVQFNWYVDGVEVHNAKTKPREEQFNSTYRVVSVLTTPHSDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSSWQEGNVFSCSVLHEALHNHYTQKSLSLSLGK

SEQ ID NO: 27 - protein, 217 aa, Artificial Sequence
  Synthetic polymer.
APEFLGGPSVFLFPPKPKDQLMISRTPEVTCVVVDVSQEDPEVQFNWYVDGVEVHNAKTKPREEQFNSTYRVVSVLTVLHQDWLSGKEYKCKVSSKGLPSSIEKTISNATGQPREPQVYTLPPSQEEMTKNQVSLTCLVKGFYPSDIAVEWESNGQPENNYKTTPPVLDSDGSFFLYSRLTVDKSRWQEGNVFSCSVLHEALHNHYTQKSLSLSLGK

SEQ ID NO: 28 - protein, 11 aa, Artificial Sequence
  Synthetic polymer.
SKYGPPCPSCP

SEQ ID NO: 29 - protein, 11 aa, Artificial Sequence
  Synthetic polymer.
SKYGPPCPPCP

SEQ ID NO: 30 - protein, 6 aa, Artificial Sequence
  Synthetic polymer.
SKYGPP

SEQ ID NO: 31 - protein, 6 aa, Artificial Sequence
  Synthetic polymer.
IEGRMD

SEQ ID NO: 32 - protein, 9 aa, Artificial Sequence
  Synthetic polymer.
GGGVPRDCG

SEQ ID NO: 33 - protein, 15 aa, Artificial Sequence
  Synthetic polymer.
IEGRMDGGGGAGGGG

SEQ ID NO: 34 - protein, 8 aa, Artificial Sequence
  Synthetic polymer.
GGGSGGGS

SEQ ID NO: 35 - protein, 12 aa, Artificial Sequence
  Synthetic polymer.
GGGSGGGGSGGG

SEQ ID NO: 36 - protein, 14 aa, Artificial Sequence
  Synthetic polymer.
EGKSSGSGSESKST

SEQ ID NO: 37 - protein, 4 aa, Artificial Sequence
  Synthetic polymer.
GGSG

SEQ ID NO: 38 - protein, 12 aa, Artificial Sequence
  Synthetic polymer.
GGSGGGSGGGSG

SEQ ID NO: 39 - protein, 15 aa, Artificial Sequence
  Synthetic polymer.
EAAAKEAAAKEAAAK

SEQ ID NO: 40 - protein, 20 aa, Artificial Sequence
  Synthetic polymer.
EAAAREAAAREAAAREAAAR

SEQ ID NO: 41 - protein, 17 aa, Artificial Sequence
  Synthetic polymer.
GGGGSGGGGSGGGGSAS

SEQ ID NO: 42 - protein, 9 aa, Artificial Sequence
  Synthetic polymer.
GGGGAGGGG

SEQ ID NO: 43 - skipped

SEQ ID NO: 44 - protein, 6 aa, Artificial Sequence
  Synthetic polymer.
GSGSGS

SEQ ID NO: 45 - protein, 10 aa, Artificial Sequence
  Synthetic polymer.
GSGSGSGSGS

SEQ ID NO: 46 - protein, 7 aa, Artificial Sequence
  Synthetic polymer.
GGGGSAS

SEQ ID NO: 47 - protein, 20 aa, Artificial Sequence
  Synthetic polymer.
APAPAPAPAPAPAPAPAPAP

SEQ ID NO: 48 - protein, 4 aa, Artificial Sequence
  Synthetic polymer.
CPPC

SEQ ID NO: 49 - protein, 5 aa, Artificial Sequence
  Synthetic polymer.
GGGGS

SEQ ID NO: 50 - protein, 10 aa, Artificial Sequence
  Synthetic polymer.
GGGGSGGGGS

SEQ ID NO: 51 - protein, 15 aa, Artificial Sequence
  Synthetic polymer.
GGGGSGGGGSGGGGS

SEQ ID NO: 52 - protein, 20 aa, Artificial Sequence
  Synthetic polymer.
GGGGSGGGGSGGGGSGGGGS

SEQ ID NO: 53 - protein, 25 aa, Artificial Sequence
  Synthetic polymer.
GGGGSGGGGSGGGGSGGGGSGGGGS

SEQ ID NO: 54 - protein, 30 aa, Artificial Sequence
  Synthetic polymer.
GGGGSGGGGSGGGGSGGGGSGGGGSGGGGS

SEQ ID NO: 55 - protein, 35 aa, Artificial Sequence
  Synthetic polymer.
GGGGSGGGGSGGGGSGGGGSGGGGSGGGGSGGGGS

SEQ ID NO: 56 - protein, 40 aa, Artificial Sequence
  Synthetic polymer.
GGGGSGGGGSGGGGSGGGGSGGGGSGGGGSGGGGSGGGGS

SEQ ID NO: 57 - protein, 16 aa, Artificial Sequence
  Synthetic polymer.
GGSGGSGGGGSGGGGS

SEQ ID NO: 58 - protein, 8 aa, Artificial Sequence
  Synthetic polymer.
GGGGGGGG

SEQ ID NO: 59 - protein, 6 aa, Artificial Sequence
  Synthetic polymer.
GGGGGG

SEQ ID NO: 60 - protein, 5 aa, Artificial Sequence
  Synthetic polymer.
EAAAK

SEQ ID NO: 61 - protein, 10 aa, Artificial Sequence
  Synthetic polymer.
EAAAKEAAAK

SEQ ID NO: 62 - protein, 15 aa, Artificial Sequence
  Synthetic polymer.
EAAAKEAAAKEAAAK

SEQ ID NO: 63 - protein, 12 aa, Artificial Sequence
  Synthetic polymer.
AEAAAKEAAAKA

SEQ ID NO: 64 - protein, 17 aa, Artificial Sequence
  Synthetic polymer.
AEAAAKEAAAKEAAAKA

SEQ ID NO: 65 - protein, 22 aa, Artificial Sequence
  Synthetic polymer.
AEAAAKEAAAKEAAAKEAAAKA

SEQ ID NO: 66 - protein, 27 aa, Artificial Sequence
  Synthetic polymer.
AEAAAKEAAAKEAAAKEAAAKEAAAKA

SEQ ID NO: 67 - protein, 46 aa, Artificial Sequence
  Synthetic polymer.
AEAAAKEAAAKEAAAKEAAAKALEAEAAAKEAAAKEAAAKEAAAKA

SEQ ID NO: 68 - protein, 5 aa, Artificial Sequence
  Synthetic polymer.
PAPAP

SEQ ID NO: 69 - protein, 18 aa, Artificial Sequence
  Synthetic polymer.
KESGSVSSEQLAQFRSLD

SEQ ID NO: 70 - protein, 12 aa, Artificial Sequence
  Synthetic polymer.
GSAGSAAGSGEF

SEQ ID NO: 71 - protein, 5 aa, Artificial Sequence
  Synthetic polymer.
GGGSE

SEQ ID NO: 72 - protein, 5 aa, Artificial Sequence
  Synthetic polymer.
GSESG

SEQ ID NO: 73 - protein, 5 aa, Artificial Sequence
  Synthetic polymer.
GSEGS

SEQ ID NO: 74 - protein, 35 aa, Artificial Sequence
  Synthetic polymer.
GEGGSGEGSSGEGSSSEGGGSEGGGSEGGGSEGGS

(19) United States
(12) Patent Application Publication
(10) Pub. No.: US 2023/0048633 A1
(43) Pub. Date: Feb. 16, 2023

(54) TGFBR2-BASED CHIMERIC PROTEINS

(71) Applicant: Shattuck Labs, Inc., Austin, TX (US)

(72) Inventors: Taylor SCHREIBER, Austin, TX (US); Casey SHUPTRINE, Austin, TX (US); George FROMM, Austin, TX (US); Suresh DE SILVA, Austin, TX (US)

(21) Appl. No.: 17/749,713

(22) Filed: May 20, 2022

Related U.S. Application Data
(60) Provisional application No. 63/191,029, filed on May 20, 2021.

(51) Int. Cl. C07K 14/71; C07K 14/705; A61P 35/00; A61P 35/02

(52) U.S. Cl. CPC C07K 14/71; C07K 14/70578; C07K 14/70503; A61P 35/00; A61P 35/02; C07K 2319/30; A61K 38/00

(57) ABSTRACT

The present invention relates, in part, to, chimeric proteins which include the extracellular domain of transforming growth factor beta receptor (TGFBR2) and their use in the treatment of diseases, such as immunotherapies for cancer and/or an inflammatory disease.

### PRIORITY

This application claims the benefit of, and priority to, U.S. Provisional Application No. 63/191,029, filed May 20, 2021, the contents of which are hereby incorporated by reference in their entirety.

### TECHNICAL FIELD

The present invention relates to, inter alia, compositions and methods, including chimeric proteins that find use in the treatment of disease, such as immunotherapies for cancer and viral infection.

### DESCRIPTION OF MATERIAL SUBMITTED ELECTRONICALLY

This application contains a sequence listing. It has been submitted electronically via EFS-Web as an ASCII text file entitled “SHK-048_SequenceListing_ST25”. The sequence listing is 49,281 bytes in size, and was created on or about Aug. 8, 2022. The sequence listing is hereby incorporated by reference in its entirety.

### BACKGROUND

Recent clinical data have demonstrated impressive patient responses to agents targeting immune coinhibitory molecules, including, for example, clinical trials that led to the approval of YERVOY, KEYTRUDA, and OPDIVO. These immunotherapies are collectively characterized as checkpoint inhibitors, and unfortunately, these therapies only provide clinical benefit for ˜15-30% of cancer patients. One potential approach to improving clinical response rates for a broader population of cancer patients includes combining a checkpoint inhibitor therapeutic with another therapy. Such combinations, when applied using multiple individual therapeutics, might lead to improved clinical benefit but are cumbersome to develop. Further, many immunotherapies are complicated by severe side effects that significantly narrow a patient's therapeutic window for treatment.

There remains a need for novel methods and compositions that provide effective immunotherapies, including consolidating multiple therapeutic mechanisms into single drugs.

### SUMMARY

Accordingly, the present invention provides, in part, compositions and methods that find use in cancer treatment by, for instance, overcoming multiple suppressive mechanisms, in the tumor microenvironment, and stimulating immune antitumor mechanisms. Similarly, the compositions and methods find use in treating an inflammatory disease.

For instance, the present invention provides, in part, chimeric proteins that, in part, operate as “traps” for TGFβ signals, thus providing, inter alia, localized reduction in TGFβ in a tumor or tumor microenvironment.

Further, the present invention provides, in part, compositions and methods that allow for dual targeting of suppressive myeloid populations by inhibiting TGFβ/TGFBR2 signaling and activation of immune cells by stimulating 4-1BB/4-1BBL, CD30/CD30L or NKG2A/HLA-E signaling. Such concurrent TGFBR2 blockade and 4-1BB/4-1BBL, CD30/CD30L or NKG2A/HLA-E agonism causes, inter alia, an overall decrease in immunosuppressive cells and a shift toward a more inflammatory milieu and an increased antitumor effect. Without being bound by theory, for example, stimulating 4-1BB/4-1BBL signaling costimulates both CD4 and CD8 T-cells, stimulating 4-1BB/4-1BBL, CD30/CD30L signaling T-cell activation, proliferation and cytokine production, and stimulating NKG2A/HLA-E signaling blocks transmission of an immune inhibitory signal to an NK cell.

In aspects, the present invention provides a heterologous chimeric protein comprising: (a) a first domain comprising a portion of transforming growth factor beta receptor (TGFBR2) that is capable of binding a TGFBR2 ligand; (b) a second domain comprising an extracellular domain of Type II transmembrane protein, the transmembrane protein being selected from 4-1BB Ligand (4-1BBL), CD30 Ligand (CD30L) and an NKG2 receptor; and (c) a linker linking the first domain and the second domain. In aspects, the present invention provides methods of treating cancer with this heterologous chimeric protein. In aspects, the present invention provides methods of treating an inflammatory disease with this heterologous chimeric protein.

In embodiments, the present invention provides a chimeric fusion protein comprising a general structure of: N terminus-(a)-(b)-(c)-C terminus, where (a) is a first domain comprising an extracellular domain of TGFBR2 that is at least 95% identical to the amino acid sequence of SEQ ID NO: 2 and is capable of binding a TGFBR2 ligand, (b) is a linker linking the first domain and the second domain and comprising a hinge-CH2-CH3 Fc domain derived from human IgG4 (e.g. at least 95% identical to the amino acid sequence of SEQ ID NO: 25, SEQ ID NO: 26, or SEQ ID NO: 27, and (c) is a second domain comprising an extracellular domain of 4-1BB Ligand (4-1BBL), that is at least 95% identical to the amino acid sequence of SEQ ID NO: 4 and is capable of binding an 4-1BB. In some embodiments, the chimeric fusion protein comprises a sequence that is at least 95% identical to the amino acid sequence of SEQ ID NO: 9. In embodiments, the present invention provides methods of treating cancer with this heterologous chimeric protein. In embodiments, the present invention provides methods of treating an inflammatory disease with this heterologous chimeric protein.

In embodiments, the present invention provides a chimeric fusion protein comprising a general structure of: N terminus-(a)-(b)-(c)-C terminus, where (a) is a first domain comprising an extracellular domain of TGFBR2 that is at least 95% identical to the amino acid sequence of SEQ ID NO: 2 and is capable of binding a TGFBR2 ligand, (b) is a linker linking the first domain and the second domain and comprising a hinge-CH2-CH3 Fc domain derived from human IgG4 (e.g. at least 95% identical to the amino acid sequence of SEQ ID NO: 25, SEQ ID NO: 26, or SEQ ID NO: 27, and (c) is a second domain comprising an extracellular domain of CD30 Ligand (CD30L), that is at least 95% identical to the amino acid sequence of SEQ ID NO: 6 and is capable of binding an CD30. In some embodiments, the chimeric fusion protein comprises a sequence that is at least 95% identical to the amino acid sequence of SEQ ID NO: 10. In embodiments, the present invention provides methods of treating cancer with this heterologous chimeric protein. In embodiments, the present invention provides methods of treating an inflammatory disease with this heterologous chimeric protein.

In embodiments, the present invention provides a chimeric fusion protein comprising a general structure of: N terminus-(a)-(b)-(c)-C terminus, where (a) is a first domain comprising an extracellular domain of TGFBR2 that is at least 95% identical to the amino acid sequence of SEQ ID NO: 2 and is capable of binding a TGFBR2 ligand, (b) is a linker linking the first domain and the second domain and comprising a hinge-CH2-CH3 Fc domain derived from human IgG4 (e.g. at least 95% identical to the amino acid sequence of SEQ ID NO: 25, SEQ ID NO: 26, or SEQ ID NO: 27, and (c) is a second domain comprising an extracellular domain of NKG2A, that is at least 95% identical to the amino acid sequence of SEQ ID NO: 8 and is capable of binding an HLA-E. In some embodiments, the chimeric fusion protein comprises a sequence that is at least 95% identical to the amino acid sequence of SEQ ID NO: 11. In embodiments, the present invention provides methods of treating cancer with this heterologous chimeric protein. In embodiments, the present invention provides methods of treating an inflammatory disease with this heterologous chimeric protein.

Any aspect or embodiment described herein can be combined with any other aspect or embodiment as disclosed herein.

### BRIEF DESCRIPTION OF THE DRAWINGS

FIG. 1A shows schematic illustrations of Type I transmembrane proteins (left protein) and Type II transmembrane proteins (right protein). FIG. 1B shows two membrane-anchored extracellular proteins, with the curved lines represents the anchoring domains; the left protein has its carboxy terminus anchored to the cell membrane and the right protein has its amino terminus anchored to the cell membrane. FIG. 1C and FIG. 1D show illustrations of chimeric proteins of the present invention; there, linkers connect the two extracellular binding domains.

FIG. 2A is a cartoon showing the structure of an illustrative chimeric protein of the present invention: TGFBR2-Fc-4-1BBL. FIG. 2B are Western blots showing characterization of the TGFBR2-Fc-4-1BBL chimeric protein. The Western blots demonstrate the chimeric protein's native state and tendency to form a multimer. Untreated samples (i.e., without a reducing agent or a deglycosylation agent, yet boiled) of the hCD86-Fc-NKG2A chimeric protein, were loaded into the lane marked as NR of each of the blots. Samples in the lane marked as R were treated with a reducing agent, β-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-TGFBR2 antibody (left blot), an anti-Fc antibody (center blot), or an anti-4-1BBL antibody (right blot).

FIG. 3A is a cartoon showing the structure of an illustrative chimeric protein of the present invention: mTGFBR2-Fc-NKG2A. FIG. 3B are Western blots showing characterization of the mouse TGFBR2-Fc-NKG2A (mTGFBR2-Fc-NKG2A) chimeric protein. The Western blots demonstrate the chimeric protein's native state and tendency to form a multimer. Untreated samples (i.e., without a reducing agent or a deglycosylation agent, yet boiled) of the mTGFBR2-Fc-NKG2A chimeric protein, were loaded into the lane marked as NR of each of the blots. Samples in the lane marked as R were treated with a reducing agent, 6-mercaptoethanol, and were boiled. Samples in the lane marked as DG were treated with a deglycosylation agent, the reducing agent, and were boiled. The lane marked as L included the protein size ladder. Each individual domain of the chimeric protein was probed using an anti-mTGFBR2 antibody (left blot), an anti-Fc antibody (center blot), or an anti-mTGFBR2 antibody (right blot).

FIG. 4 shows the results of ELISA assays demonstrating the presence of Fc domain in the TGFBR2-Fc-CD30L and TGFBR2-Fc-4-1BBL chimeric proteins. Anti-mFc antibody was coated on plates and increasing amounts of the TGFBR2-Fc-CD30L and TGFBR2-Fc-4-1BBL chimeric proteins or mFc were added to the plates for capture by the plate-bound anti-mFc antibody. The mFc IgG was used as a positive control. The binding was detected using an anti-mFc HRP.

FIG. 5 shows the results of ELISA assays demonstrating the dose-dependent binding of the Fc domain of the human CD86-Fc-NKG2A (hCD86-Fc-NKG2A) and human TGFBR2-Fc-NKG2A (hTGFBR2-Fc-NKG2A) chimeric proteins to anti-human Fc antibody. Anti-human IgG was coated on plates and increasing amounts of the indicated chimeric proteins were added to the plates. Human IgG (hIgG) and the hCD86-Fc-NKG2A chimeric protein were used as positive controls, and mouse CD80-Fc-NKG2A (mCD80-Fc-NKG2A) was used as a negative control. The binding was detected using an anti-human Fcgamma HRP.

FIG. 6 shows the results of ELISA assays demonstrating the binding of the TGFBR2-Fc-4-1BBL chimeric protein to 4-1BB. Recombinant 4-1BB-His protein was coated on plates. Increasing amounts of the TGFBR2-Fc-4-1BBL chimeric protein, or positive control protein comprising Fc-4-1BBL (Control-Fc-4-1BBL) were added for capture by the plate-bound 4-1BB-His protein. Binding was detected using an anti mFc-HRP antibody.

FIG. 7 shows the results of ELISA assays demonstrating the dose-dependent binding of the hTGFBR2-Fc-NKG2A and hSLAMF6-Fc-NKG2A chimeric proteins to HLA-E. The hSLAMF6-Fc-NKG2A chimeric protein was used as a positive control, and a chimeric protein comprising ECD of a type II transmembrane protein other than NKG2A was used as a negative control. Increasing amounts of the indicated chimeric proteins were coated on plates and detected using an anti-human HLA-E-His

FIG. 8 shows the results of ELISA assays demonstrating the binding of the TGFBR2-Fc-4-1BBL, TGFBR2-Fc-CD30L, and TGFBR2-Fc-NKG2A chimeric proteins to TGFβ1. TGFβ1 was coated on plates, the TGFBR2-Fc-4-1BBL, TGFBR2-Fc-CD30L, and TGFBR2-Fc-NKG2A chimeric proteins for capture by the plate-bound TGFβ1 protein and detected using an anti mFc-HRP antibody.

FIG. 9 shows the results of ELISA assays demonstrating the dose-dependent binding of the mTGFBR2-Fc-NKG2A chimeric protein to mTGFβ1. mTGFβ1 was coated on plates and detected using mTGFBR2-Fc-NKG2A, or mCD80-Fc-NKG2A, which was used as a negative control.

FIG. 10 shows the binding kinetics of the mTGFBR2-Fc-4-1BBL chimeric protein to 4-1BB-His protein as determined using the Octet system (ForteBio). Recombinant 4-1BB-His protein was immobilized and detected using the mTGFBR2-Fc-4-1BBL chimeric protein, Control-Fc-4-1BBL or recombinant mouse 4-1 BB Ligand protein (R&D Systems, Cat. No. 1246-4L). As shown, the mTGFBR2-Fc-4-1BBL chimeric protein bound to m4-1BB-His with a higher K_{D} than the commercially available recombinant mouse 4-1 BB Ligand protein.

FIG. 11 shows the binding kinetics of the TGFBR2-Fc-4-1BBL, TGFBR2-Fc-CD30L, and TGFBR2-Fc-NKG2A chimeric proteins to TGFβ1 protein as determined using the Octet system. Varying concentrations of the recombinant mouse TGFβ1 protein (BioLegend 763104) were covalently linked to amine-reactive (ARG2, ForteBio) OCTET tips and detected using the TGFBR2-Fc-4-1BBL, TGFBR2-Fc-CD30L, and TGFBR2-Fc-NKG2A chimeric proteins (5 μg/ml each). The data obtained with 100 nM recombinant Mouse TGFβ1 protein are shown. These data demonstrate that the TGFBR2-Fc-4-1BBL, TGFBR2-Fc-CD30L, and TGFBR2-Fc-NKG2A chimeric proteins bind the recombinant Mouse TGFβ1 protein with similar kinetics.

FIG. 12 shows the results a TGFβ1 sequestration assay. A TGFβ1 expressing tumor cell line EO771 was incubated with the indicated amounts of an anti-TGFB1 antibody, mTGFBR2-fc-4-1BBL, mTGFBR2-fc-CD30L, and mTGFBR2-fc-NKG2a. After 8 hours of incubation, the media were collected and TGFβ1 in the culture supernatant was assayed using ELISA.

FIG. 13 demonstrates the mTGFBR2-Fc-4-1BBL chimeric protein can completely inhibit TGFβ1 signaling in reporter cells. HEK BLUE TGF-β cells (Invivogen) cells were exposed to an anti-TGFB1 antibody or the mTGFBR2-Fc-4-1BBL chimeric protein for 24 hours. Aliquots of culture supernatant were removed and assayed for TGFβ signaling. Student T-Test was performed. **p<0.01, ***p<0.001.

FIG. 14A and FIG. 14B show the binding of the TGFBR2-Fc-4-1BBL chimeric protein to wild type (WT) CHO-K1 cells (FIG. 14A) or the CHO-K1/4-1BB cells (FIG. 14B) as measured by flow cytometry. The dose dependent shifts in the CHO-K1/4-1BB cells, but not WT CHO-K1 cells illustrate dose dependent binding of the mTGFBR2-Fc-4-1BBL chimeric protein to 4-1BB expressed by the CHO-K1/4-1BB cells.

FIG. 15 shows the quantitation of the binding of the TGFBR2-Fc-4-1BBL chimeric protein and Control-Fc-4-1BBL to the CHO-K1/4-1BB cells in comparison to WT CHO-K1 cells as measured by flow cytometry.

FIG. 16 demonstrates that the TGFBR2-Fc-4-1BBL chimeric protein induces apoptosis of antigen positive (EO771-OVA+) target cells mediated by the antigen activated T cells (OT-1 naïve T cells) in a dose-dependent manner. Increasing amounts of the TGFBR2-Fc-4-1BBL chimeric protein was incubated with OT-1 naïve T cells (effector cells) and OVA+ cells (target cells). Apoptosis was assessed by measuring caspase 3/7 activity.

FIG. 17 shows demonstrates that the TGFBR2-Fc-4-1BBL and TGFBR2-Fc-CD30L chimeric proteins induces apoptosis of antigen positive (EO771-OVA+) target cells mediated by the antigen activated T cells (OT-1 naïve T cells). The indicated amounts of the TGFBR2-fc-4-1BBL and TGFBR2-Fc-CD30L chimeric proteins was incubated with OT-1 naïve T cells (effector cells) and OVA+ cells (target cells). Apoptosis was assessed by measuring caspase 3/7 activity.

FIG. 18 shows the results of luciferase assays illustrating that the TGFBR2-Fc-NKG2A chimeric protein activates Qa1 signaling in a dose dependent manner. The CHO-K1/Qa1 cells used in these assays harbor an NFκB-luciferase reporter that is sensitive to the binding of a ligand to the Qa1 protein that the cells express. WT CHO-K1 cells were used as a negative control. Increasing amounts of the TGFBR2-Fc-NKG2A chimeric protein was incubated with the CHO-K1/Qa1 cells, or WT CHO-K1 cells, and the activation of the Qa1 was measured by a luciferase assay.

FIG. 19A and FIG. 19B demonstrate the efficacy of the TGFBR2-Fc-4-1BBL chimeric protein, alone or in combination with an anti-PD1 antibody against allografts of the triple negative murine breast cancer cell line EO771. C57Bl/6 mice were inoculated with EO771 cells. After the tumors were established, the mice were randomly assigned to the following treatment groups: (1) untreated, (2) an anti-PD1 antibody (clone RMP1-14), (3) an anti-TGFβ1 antibody (clone 1D11.16.8), (4) 300 μg/mouse of the TGFBR2-Fc-NKG2A chimeric protein, and (5) the anti-PD1 antibody 300 μg/mouse of the TGFBR2-Fc-NKG2A chimeric protein. Tumor volumes were measured on indicated days. FIG. 19A shows the tumor volumes plotted as a function of time.

FIG. 19B shows the tumor volumes on day 17. * denotes p 0.05 between the indicated groups, and ** denotes p 0.01 between the indicated groups.

FIG. 20A and FIG. 20B demonstrate the efficacy of the TGFBR2-Fc-NKG2A chimeric protein against allografts of the murine myelomonocytic leukemia cell line WEHI-3. Balb/c mice were inoculated with WEHI-3 cells. After the tumors were established, the mice were randomly assigned to the following treatment groups: (1) untreated, (2) 100 μg/mouse of an anti-PD1 antibody (Bioxcell clone RMP1-14), (3) 100 μg/mouse of an anti-NKG2A antibody (BioXcell clone 20D5) and (4) 300 μg/mouse of the TGFBR2-Fc-NKG2A chimeric protein. The mice were treated on days 0, 2, 4, 6, 8 and 10 post-inoculation. The mice were treated six times, two days apart. Tumor volumes were measured on indicated days. FIG. 20A shows the tumor volumes plotted as a function of time. FIG. 20B shows the tumor volumes on day 18. * denotes p≤0.05 between the indicated groups, and ** denotes p≤0.01 between the indicated groups.

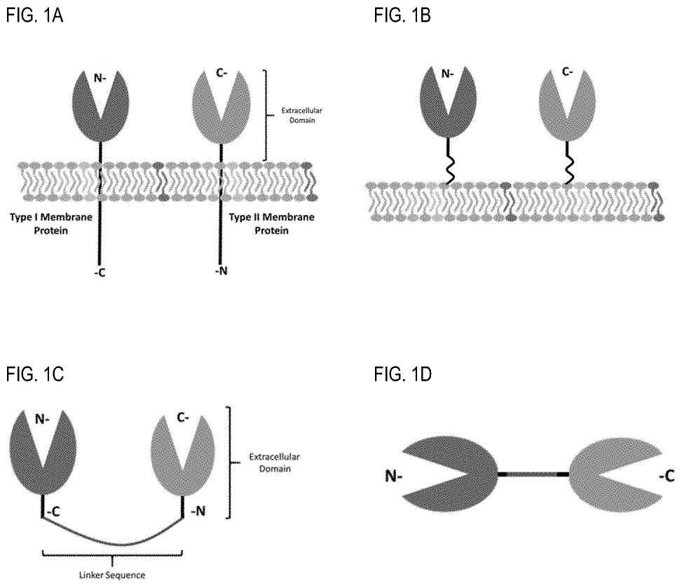

### DETAILED DESCRIPTION

The present invention is based, in part, on the discovery of engineered chimeric proteins comprising a first domain comprising a portion of transforming growth factor beta receptor (TGFBR2) that is capable of binding a TGFBR2 ligand. In embodiments, the chimeric protein further comprises a second domain comprising a portion of −1BB Ligand (4-1BBL) that is capable of binding 4-1BB, CD30 Ligand (CD30L) that is capable of binding CD30 and an NKG2 receptor that is capable of binding HLA-E. In embodiments, the first domain and the second domain are connected by a linker. In embodiments, the present chimeric protein provides an immune stimulatory signal, for example, capable of activating macrophages and antigen presenting cells, while providing a localized trap for an inhibitory signal that could otherwise shift the balance toward immunosuppression (e.g., TGFβ (e.g. TGFβ 1 and/or TGFβ3)). Embodiments of the invention thereby provide for the effective treatment of cancers and/or inflammatory diseases.

### Chimeric Proteins

In aspects, the present invention provides a chimeric protein of a general structure of: N terminus-(a)-(b)-(c)-C terminus, wherein: (a) is a first domain comprising an extracellular domain of a Type I transmembrane protein, the transmembrane protein being transforming growth factor, beta receptor II (TGFBRII), (b) is a linker comprising at least one cysteine residue capable of forming a disulfide bond, and (c) is a second domain comprising an extracellular domain of Type II transmembrane protein, the transmembrane protein being selected from 4-1BB Ligand (4-1BBL), CD30 Ligand (CD30L) and an NKG2 receptor, wherein: the linker connects the first domain and the second domain and optionally comprises one or more joining linkers. TGFBR2 is a member of the serine/threonine protein kinase family and the TGFB receptor subfamily. It is a single-pass type I membrane protein which functions as a receptor for the transforming growth factor beta (TGFβ). Binding of TGFBR2 to TGFβ has an adverse effect on anti-tumor immunity and significantly inhibits host tumor immune surveillance. TGFβ suppresses the activity of cytotoxic T lymphocytes (CTLs), through transcriptional repression of genes encoding multiple key proteins, such as perforin, granzymes and cytotoxins. TGFβ also has a significant negative impact on the differentiation, clonal expansion and function of both CD4+ and CD8+ T cells. In addition, TGFβ induces Foxp3 and generates induced regulatory T cells (Tregs). Furthermore, TGFβ mediates the lineage-specific differentiation of the Th17 cells. TGFβ also inhibits NK-cell proliferation and function. In addition, exogenous administration of TGFβ suppressed B-cell proliferation and Ig secretion. Thus, TGFβ signaling immunosuppressive and pro-tumorigenic.

Accordingly, in aspects, the present invention provides a chimeric protein comprising a domain of TGFBR2 that traps and neutralizes TGFβ. In embodiments, the present invention relates to chimeric proteins engineered to comprise a domain, e.g., the extracellular domain, of the immune inhibitory receptor transforming growth factor, beta receptor II (TGFBR2). In embodiments, the first domain comprises substantially all the extracellular domain of TGFBRII. In embodiments, the TGFBRII binds to a transforming growth factor (TGFβ). In embodiments, the TGFβ is TGFβ3 and/or TGFβ1. In embodiments, the binding to the TGFβ inhibits signaling by TGFβ3 and/or TGFβ1.

In embodiments, the present chimeric protein comprises a domain, e.g., the extracellular domain, of human TGFBR2. The human TGFBR2 comprises the amino acid sequence of SEQ ID NO: 1:

TIPPHVQKSVNNDMIVTDNNGAVKFPQLCKFCDVR
FSTCDNQKSCMSNCSITSICEKPQEVCVAVWRKND
ENITLETVCHDPKLPYHDFILEDAASPKCIMKEKK
KPGETFFMCSCSSDECNDNIIFSEEYNTSNPDLLL
VIFQVTGISLLPPLGVAISVIIIFYCYRVNRQQKL
SSTWETGKTRKLMEFSEHCAIILEDDRSDISSTCA
NNINHNTELLPIELDTLVGKGRFAEVYKAKLKQNT
SEQFETVAVKIFPYEEYASWKTEKDIFSDINLKHE
NILQFLTAEERKTELGKQYWLITAFHAKGNLQEYL
TRHVISWEDLRKLGSSLARGIAHLHSDHTPCGRPK
MPIVHRDLKSSNILVKNDLTCCLCDFGLSLRLDPT
LSVDDLANSGQVGTARYMAPEVLESRMNLENVESF
KQTDVYSMALVLWEMTSRCNAVGEVKDYEPPFGSK
VREHPCVESMKDNVLRDRGRPEIPSFWLNHQGIQM
VCETLTECWDHDPEARLTAQCVAERFSELEHLDRL
SGRSCSEEKIPEDGSLNTTK

The amino acid sequence of the extracellular domain comprising amino acids 1 to 144 (SEQ ID NO: 2):

TIPPHVQKSVNNDMIVTDNNGAVKFPQLCKFCDVR
FSTCDNQKSCMSNCSITSICEKPQEVCVAVWRKND
ENITLETVCHDPKLPYHDFILEDAASPKCIMKEKK
KPGETFFMCSCSSDECNDNIIFSEEYNTSNPDLLL
VIFQ

In embodiments, the present chimeric protein comprises the extracellular domain, of human TGFBR2, which has the amino acid sequence of SEQ ID NO: 2. In embodiments, the present chimeric proteins may comprise the extracellular domain of TGFBR2 as described herein, or a variant or a functional fragment thereof. For instance, the chimeric protein may comprise a sequence of the extracellular domain of TGFBR2 as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of the extracellular domain of TGFBR2 as described herein.

The structure of TGFBR2 is described, for example, in Hart, et al., “Crystal structure of the human TbetaR2 ectodomain—TGF-beta3 complex.” Nat Struct Biol 9: 203-208 (2002); Deep et al., “Solution structure and backbone dynamics of the TGF-beta type II receptor extracellular domain.” Biochemistry 42: 10126-10139 (2003). Derivatives of TGFBR2 can be prepared based upon available TGFBR2 structures.

In embodiments, the present chimeric proteins may comprise a variant extracellular domain of TGFBR2 in which the signal peptide (e.g., as provided in SEQ ID NO: 1) is replaced with an alternative signal peptide. In embodiments, the present chimeric protein may comprise a variant extracellular domain of TGFBR2 which is expressed from a cDNA that has been codon-optimized for expression in protein producing cells such as Chinese Hamster Ovary (CHO) or human embryonic kidney (HEK) cells. In embodiments, the present chimeric protein may comprise a variant extracellular domain of TGFBR2 which is based on an alternatively spliced variant or isoform of TGFBR2.

In embodiments, an extracellular domain of TGFBR2 refers to a portion of the protein which is capable of interacting with the extracellular environment. In embodiments, the extracellular domain of TGFBR2 is the entire amino acid sequence of the protein which is external of a cell or the cell membrane. In embodiments, the extracellular domain of TGFBR2 is a portion of an amino acid sequence of the protein which is external of a cell or the cell membrane and is needed for signal transduction and/or ligand binding as may be assayed using methods known in the art (e.g., in vitro ligand binding and/or cellular activation assays).

In embodiments, the extracellular domain of TGFBR2 refers to a portion of the protein which is capable for binding to transforming growth factor beta (TGFβ). In embodiments, the chimeric protein binds to human TGFβ with a K_{D} of less than about 1 μM, about 900 nM, about 800 nM, about 700 nM, about 600 nM, about 500 nM, about 400 nM, about 300 nM, about 200 nM, about 150 nM, about 130 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human TGFβ with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry).

In embodiments, the extracellular domain of TGFBR2 refers to a portion of the protein which is capable for binding to TGFβ1. In embodiments, the chimeric protein binds to human TGFβ (E.G. TGFB 1 AND/OR TGFB3) with a K_{D} of less than about 1 pM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to TGFB (E.G. TGFB 1 AND/OR TGFB3) with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human TGFβ with a K_{D} of from about 100 pM to about 600 pM.

In embodiments, the extracellular domain of TGFBR2 refers to a portion of the protein which is capable for binding to TGFβ2. In embodiments, the chimeric protein binds to human TGFB (E.G. TGFB 1 AND/OR TGFB3) with a K_{D} of less than about 1 pM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to TGFB (E.G. TGFB 1 AND/OR TGFB3) with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human TGFβ with a K_{D} of from about 100 pM to about 600 pM.

In embodiments, the extracellular domain of TGFBR2 refers to a portion of the protein which is capable for binding to TGFβ3. In embodiments, the chimeric protein binds to human TGFB (E.G. TGFB 1 AND/OR TGFB3) with a K_{D} of less than about 1 pM, about 900 nM, about 800 nM, about 700 nM, about 600 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to TGFB (E.G. TGFB 1 AND/OR TGFB3) with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human TGFβ with a K_{D} of from about 100 pM to about 600 pM.

In embodiments, the present chimeric protein further comprises a domain, e.g., the extracellular domain, of the immune stimulatory molecule 4-1BB ligand (4-1BBL). 4-1BBL is a type II transmembrane protein belonging to the Tumor Necrosis Factor (TNF) superfamily. In embodiments, the second domain is 4-1BBL. In embodiments, the second domain comprises substantially all the extracellular domain of 4-1BBL. In embodiments, the second domain is capable of binding 4-1BB (also known as cluster of differentiation 137 (CD137) or tumor necrosis factor ligand superfamily member 9 (TNFSF9)). In embodiments, the binding to 4-1BB costimulates both CD4 and CD8 T-cells. 4-1BBL is also known as cluster of differentiation 137 ligand (CD137L). Thus, throughout this disclosure, 4-1BBL and CD137L are synonymous, when referenced alone and/or when referenced in context of a chimeric protein, thus, for example, TGFBR2-Fc-4-1BBL is the same chimeric protein as TGFBR2-Fc-CD137L.

4-1BB ligand (4-1BBL) binds to the 4-1BB receptor on activated T Lymphocytes and antigen-presenting cells (APC). 4-1BB signaling is believed to follow an immune synapse, formed by 4-1BB+ lymphocytes and 4-1BBL+antigen-presenting cells. For example, 4-1BBL binding induces B cell proliferation and immunoglobulin production. T cells are the major 4-1BB-expressing cells and may engage 4-1BBL on macrophages and or APCs for their activation. CD8+ T cells release IL-13 as well as IFN-γ through 4-1BB signaling. In embodiments, the present chimeric protein comprises a domain, e.g., the extracellular domain, of human 4-1BBL. The human 4-1BBL comprises the amino acid sequence of SEQ ID NO: 3:

MEYASDASLDPEAPWPPAPRARACRVLPWALVAGL
LLLLLLAAACAVFLACPWAVSGARASPGSAASPRL
REGPELSPDDPAGLLDLRQGMFAQLVAQNVLLIDG
PLSWYSDPGLAGVSLTGGLSYKEDTKELVVAKAGVY
YVFFQLELRRVVAGEGSGSVSLALHLQPLRSAAGA
AALALTVDLPPASSEARNSAFGFQGRLLHLSAGQR
LGVHLHTEARARHAWQLTQGATVLGLFRVTPEIPA
GLPSPRSE

The amino acid sequence of extracellular domain human 4-1BBL (amino acids 50-254 of SEQ ID NO 3) is SEQ ID NO: 4:

ACPWAVSGARASPGSAASPRLREGPELSPDDPAGL
LDLRQGMFAQLVAQNVLLIDGPLSWYSDPGLAGVS
LTGGLSYKEDTKELVVAKAGVYYVFFQLELRRVVAG
EGSGSVSLALHLQPLRSAAGAAALALTVDLPPASS
EARNSAFGFQGRLLHLSAGQRLGVHLHTEARARHA
WQLTQGATVLGLFRVTPEIPAGLPSPRSE

In embodiments, the present chimeric protein comprises the extracellular domain of human 4-1BBL which has the amino acid sequence of SEQ ID NO: 4. In embodiments, the present chimeric proteins may comprise the extracellular domain of 4-1BBL as described herein, or a variant or functional fragment thereof. For instance, the chimeric protein may comprise a sequence of the extracellular domain of 4-1BBL as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of 4-1BBL as described herein.

4-1BBL derivatives can be constructed from available structural data, including that described by Won et al., “The structure of the trimer of human 4-1BB ligand is unique among members of the tumor necrosis factor superfamily.” J. Biol. Chem. 285: 9202-9210 (2010).

In embodiments, the present chimeric proteins may comprise a variant extracellular domain of 4-1BBL in which the signal peptide (e.g., as provided in SEQ ID NO: 3) is replaced with an alternative signal peptide. In embodiments, the present chimeric protein may comprise a variant extracellular domain of 4-1BBL which is expressed from a cDNA that has been codon-optimized for expression in protein producing cells such as Chinese Hamster Ovary (CHO) or HEK cells.

In embodiments, the extracellular domain of 4-1BBL refers to a portion of protein which is capable of interacting with the extracellular environment. In embodiments, the extracellular domain of 4-1BBL is the entire amino acid sequence of the protein which is external of a cell or the cell membrane. In embodiments, the extracellular domain of 4-1BBL is a portion of an amino acid sequence of the protein which is external of a cell or the cell membrane and is needed for signal transduction and/or ligand binding as may be assayed using methods know in the art.

In embodiments, the extracellular domain of 4-1BBL refers to a portion of the protein which is capable for binding to the 4-1BB receptor. Similar to other TNF superfamily members, membrane-bound 4-1BBL exists as a homotrimer. 4-1BBL binds to 4-1BB, a member of the TNF receptor superfamily that is expressed predominantly on antigen presenting cells.

In embodiments, the chimeric protein of the invention binds to human 4-1BB with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human 4-1BB with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human 4-1BB with a K_{D} of from about 300 pM to about 700 pM.

In embodiments, the present chimeric protein further comprises a domain, e.g., the extracellular domain, of the immune stimulatory molecule CD30 ligand (CD30L, also known as CD154). CD30L is a type II transmembrane protein belonging to the Tumor Necrosis Factor (TNF) superfamily. CD30L expressed on activated T cells, B cells, monocytes and granulocytes. CD30 has been described as a marker of memory T cells but can also be expressed by activated B cells and effector T cells. CD30 ligation by CD30L mediates pleiotropic effects including cell proliferation, activation, differentiation and cell death by apoptosis

In embodiments, the second domain is CD30L. In embodiments, the second domain comprises substantially all the extracellular domain of CD30L. In embodiments, the second domain is capable of binding CD30 (also known as Ki-1 antigen or tumor necrosis factor ligand superfamily member 8 (TNFSF8)). In embodiments, the binding to CD30 enhances T-cell activation, proliferation and cytokine production. CD30L is also known as cluster of differentiation 153 (CD153). Thus, throughout this disclosure, CD30L and CD153 are synonymous, when referenced alone and/or when referenced in context of a chimeric protein, thus, for example, TGFBR2-Fc-CD30L is the same chimeric protein as TGFBR2-Fc-CD153.

In embodiments, the present chimeric protein comprises a domain, e.g., the extracellular domain, of human CD30L. The human CD30L comprises the amino acid sequence of SEQ ID NO: 5:

MDPGLQQALNGMAPPGDTAMHVPAGSVASHLGTTS
RSYFYLTTATLALCLVFTVATIMVLVVQRTDSIPN
SPDNVPLKGGNCSEDLLCILKRAPFKKSWAYLQVA
KHLNKTKLSWNKDGILHGVRYQDGNLVIQFPGLYF
IICQLQFLVQCPNNSVDLKLELLINKHIKKQALVT
VCESGMQTKHVYQNLSQFLLDYLQVNTTISVNVDT
FQYIDTSTFPLENVLSIFLYSNSD

The amino acid sequence of extracellular domain human CD30L (amino acids 63-234 of SEQ ID NO 5) is SEQ ID NO: 6:

QRTDSIPNSPDNVPLKGGNCSEDLLCILKRAPFKK
SWAYLQVAKHLNKTKLSWNKDGILHGVRYQDGNLV
IQFPGLYFIICQLQFLVQCPNNSVDLKLELLINKH
IKKQALVTVCESGMQTKHVYQNLSQFLLDYLQVNT
TISVNVDTFQYIDTSTFPLENVLSIFLYSNSD

In embodiments, the present chimeric protein comprises the extracellular domain of human CD30L which has the amino acid sequence of SEQ ID NO: 6. In embodiments, the present chimeric proteins may comprise the extracellular domain of CD30L as described herein, or a variant or functional fragment thereof. For instance, the chimeric protein may comprise a sequence of the extracellular domain of CD30L as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of CD30L as described herein.

In embodiments, the present chimeric proteins may comprise a variant extracellular domain of CD30L in which the signal peptide (e.g., as provided in SEQ ID NO: 5) is replaced with an alternative signal peptide. In embodiments, the present chimeric protein may comprise a variant extracellular domain of CD30L which is expressed from a cDNA that has been codon-optimized for expression in protein producing cells such as Chinese Hamster Ovary (CHO) or HEK cells.

In embodiments, the extracellular domain of CD30L refers to a portion of protein which is capable of interacting with the extracellular environment. In embodiments, the extracellular domain of CD30L is the entire amino acid sequence of the protein which is external of a cell or the cell membrane. In embodiments, the extracellular domain of CD30L is a portion of an amino acid sequence of the protein which is external of a cell or the cell membrane and is needed for signal transduction and/or ligand binding as may be assayed using methods know in the art.

In embodiments, the extracellular domain of CD30L refers to a portion of the protein which is capable for binding to the CD30 receptor. Similar to other TNF superfamily members, membrane-bound CD30L exists as a homotrimer. CD30L binds to CD30, a member of the TNF receptor superfamily.

In embodiments, the chimeric protein of the invention binds to human CD30 with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human CD30 with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human CD30 with a K_{D} of from about 300 pM to about 700 pM.

In embodiments, the second domain is the NKG2 receptor selected from NKG2A, NKG2B, NKG2C, NKG2D, NKG2E, NKG2F, and NKG2H. In embodiments, the member of the NKG2 receptor is NKG2A. In embodiments, the second domain comprises substantially all the extracellular domain of NKG2A. In embodiments, the second domain is capable of binding an NKG2A ligand. In embodiments, the NKG2A ligand is HLA class I histocompatibility antigen, alpha chain E, which is also known as MHC class I antigen E (HLA-E). In embodiments, binding to the NKG2A ligand blocks transmission of an immune inhibitory signal to an NK cell. NKG2A is also known as cluster of differentiation 153 (CD153). Thus, throughout this disclosure, NKG2A and CD159a are synonymous, when referenced alone and/or when referenced in context of a chimeric protein, thus, for example, TGFBR2-Fc-NKG2A is the same chimeric protein as TGFBR2-Fc-CD159a.

In embodiments, the present chimeric protein further comprises a domain, e.g., the extracellular domain, of the immune stimulatory molecule natural killer group 2A (NKG2A), also known as CD154. NKG2A is a type II transmembrane protein expressed on the natural killer (NK) cells. NK cells avoid the killing of healthy autologous cells through MHC-I specific inhibitory receptor proteins as NKG2A. Ligation of NKG2A and its receptor HLA-E expressed on normal cells suppresses signaling activation, thereby avoiding the destruction of normal bystander cells. Tumor cells (hematological as well as solid tumors), in order to avoid killing by NK cells, have shown upregulation of HLA-E expression. In various cancers, poor prognosis has been associated with HLA-E upregulation.

In embodiments, the present chimeric protein comprises a domain, e.g., the extracellular domain, of human NKG2A. The human NKG2A comprises the amino acid sequence of SEQ ID NO: 7:

MDNQGVIYSDLNLPPNPKRQQRKPKGNKNSILATE
QEITYAELNLQKASQDFQGNDKTYHCKDLPSAPEK
LIVGILGIICLILMASVVTIVVIPSTLIQRHNNSS
LNTRTQKARHCGHCPEEWITYSNSCYYIGKERRTW
EESLLACTSKNSSLLSIDNEEEMKFLSIISPSSWI
GVFRNSSHHPWVTMNGLAFKHEIKDSDNAELNCAV
LQVNRLKSAQCGSSHYHCKHKL

The amino acid sequence of extracellular domain human NKG2A (amino acids 94-233 of SEQ ID NO 7) is SEQ ID NO: 8:

PSTLIQRHNNSSLNTRTQKARHCGHCPEEWITYSN
SCYYIGKERRTWEESLLACTSKNSSLLSIDNEEEM
KFLSIISPSSWIGVFRNSSHHPWVTMNGLAFKHEI
KDSDNAELNCAVLQVNRLKSAQCGSSIIYHCKHKL

In embodiments, the present chimeric protein comprises the extracellular domain of human NKG2A which has the amino acid sequence of SEQ ID NO: 8. In embodiments, the present chimeric proteins may comprise the extracellular domain of NKG2A as described herein, or a variant or functional fragment thereof. For instance, the chimeric protein may comprise a sequence of the extracellular domain of NKG2A as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the extracellular domain of NKG2A as described herein.

NKG2A derivatives can be constructed from available structural data, including that described by Sullivan et al., “The Heterodimeric Assembly of the CD94-NKG2 Receptor Family and Implications for Human Leukocyte Antigen-E Recognition” Immunity 27:900-911 (2007); Kaiser et al., “Structural basis for NKG2A/CD94 recognition of HLA-E.” Proc. Natl. Acad. Sci. Usa, 105:6696-6701 (2008).

In embodiments, the present chimeric proteins may comprise a variant extracellular domain of NKG2A in which the signal peptide (e.g., as provided in SEQ ID NO: 3) is replaced with an alternative signal peptide. In embodiments, the present chimeric protein may comprise a variant extracellular domain of NKG2A which is expressed from a cDNA that has been codon-optimized for expression in protein producing cells such as Chinese Hamster Ovary (CHO) or HEK cells.

In embodiments, the extracellular domain of NKG2A refers to a portion of protein which is capable of interacting with the extracellular environment. In embodiments, the extracellular domain of NKG2A is the entire amino acid sequence of the protein which is external of a cell or the cell membrane. In embodiments, the extracellular domain of NKG2A is a portion of an amino acid sequence of the protein which is external of a cell or the cell membrane and is needed for signal transduction and/or ligand binding as may be assayed using methods know in the art.

In embodiments, the extracellular domain of NKG2A refers to a portion of the protein which is capable for binding to HLA-E.

In embodiments, the chimeric protein of the invention binds to human HLA-E with a K_{D} of less than about 1 μM, about 900 nM, about 800 nM, about 700 nM, about 600 nM, about 550 nM, about 530 nM, about 500 nM, about 400 nM, about 300 nM, about 200 nM, about 100 nM, about 90 nM, about 80 nM, about 70 nM, about 60 nM, about 55 nM, about 50 nM, about 45 nM, about 40 nM, about 35 nM, about 30 nM, about 25 nM, about 20 nM, about 15 nM, about 10 nM, or about 5 nM, or about 1 nM (as measured, for example, by surface plasmon resonance or biolayer interferometry). In embodiments, the chimeric protein binds to human HLA-E with a K_{D} of less than about 1 nM, about 900 pM, about 800 pM, about 700 pM, about 600 pM, about 500 pM, about 400 pM, about 300 pM, about 200 pM, about 100 pM, about 90 pM, about 80 pM, about 70 pM, about 60 pM about 55 pM about 50 pM about 45 pM, about 40 pM, about 35 pM, about 30 pM, about 25 pM, about 20 pM, about 15 pM, or about 10 pM, or about 1 pM (as measured, for example, by surface plasmon resonance or biolayer interferometry). In embodiments, the chimeric protein binds to human HLA-E with a K_{D} of from about 300 pM to about 700 pM.

In embodiments, the chimeric protein of the present invention comprises an extracellular domain of TGFBR2 (SEQ ID NO: 2).

In embodiments, the chimeric protein of the present invention comprises an extracellular domain of 4-1BBL (SEQ ID NO: 4).

In embodiments, the chimeric protein of the present invention comprises an extracellular domain of CD30L (SEQ ID NO: 6).

In embodiments, the chimeric protein of the present invention comprises an extracellular domain of NKG2A (SEQ ID NO: 8).

In embodiments, the chimeric protein of the present invention comprises an extracellular domain of TGFBR2 (SEQ ID NO: 2) and the extracellular domain of 4-1BBL (SEQ ID NO: 4).

In embodiments, the chimeric protein of the present invention comprises an extracellular domain of TGFBR2 (SEQ ID NO: 2) and the extracellular domain of CD30L (SEQ ID NO: 6).

In embodiments, the chimeric protein of the present invention comprises an extracellular domain of TGFBR2 (SEQ ID NO: 2) and the extracellular domain of NKG2A (SEQ ID NO: 8).

In embodiments, the chimeric protein of the present invention comprises the hinge-CH2-CH3 domain from a human IgG4 antibody sequence (SEQ ID NO: 25, SEQ ID NO: 26, or SEQ ID NO: 27).

In embodiments, the chimeric protein of the present invention comprises an extracellular domain of TGFBR2 and the extracellular domain of 4-1BBL, using the hinge-CH2-CH3 domain from a human IgG4 antibody sequence as a linker (this TGFBR2-Fc-4-1BBL chimera is SEQ ID NO: 9):

(SEQ ID NO: 9)
TIPPHVQKSVNNDMIVTDNNGAVKFPQLCKFCDVR
FSTCDNQKSCMSNCSITSICEKPQEVCVAVWRKND
ENITLETVCHDPKLPYHDFILEDAASPKCIMKEKK
KPGETFFMCSCSSDECNDNIIFSEEYNTSNPDLLL
VIFQSKYGPPCPPCPAPEFLGGPSVFLFPPKPKDQ
LMISRTPEVTCVVVDVSQEDPEVQFNWYVDGVEVHN
AKTKPREEQFNSTYRVVSVLTVLHQDWLSGKEYKC
KVSSKGLPSSIEKTISNATGQPREPQVYTLPPSQE
EMTKNQVSLTCLVKGFYPSDIAVEWESNGQPENNY
KTTPPVLDSDGSFFLYSRLTVDKSRWQEGNVFSCS
VLHEALHNHYTQKSLSLSLGKIEGRMDACPWAVSG
ARASPGSAASPRLREGPELSPDDPAGLLDLRQGMF
AQLVAQNVLLIDGPLSWYSDPGLAGVSLTGGLSYK
EDTKELVVAKAGVYYVFFQLELRRVVAGEGSGSVS
LALHLQPLRSAAGAAALALTVDLPPASSEARNSAF
GFQGRLLHLSAGQRLGVHLHTEARARHAWQLTQGA
TVLGLFRVTPEIPAGLPSPRSE

In embodiments, the chimeric protein of the present invention comprises SEQ ID NO: 9, i.e., monomeric TGFBR2-Fc-4-1BBL chimeric protein, or a variant or functional fragment thereof.

In embodiments, the chimeric protein may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%, or 100% sequence identity with the amino acid sequence of any one of SEQ ID NO: 9.

In embodiments, the chimeric protein of the present invention comprises an extracellular domain of TGFBR2 and the extracellular domain of 4-1BBL, using the hinge-CH2-CH3 domain from a human IgG4 antibody sequence as a linker (this TGFBR2-Fc-CD30L chimera is SEQ ID NO: 10):

(SEQ ID NO: 10)
TIPPHVQKSVNNDMIVTDNNGAVKFPQLCKFCDVR
FSTCDNQKSCMSNCSITSICEKPQEVCVAVWRKND
ENITLETVCHDPKLPYHDFILEDAASPKCIMKEKK
KPGETFFMCSCSSDECNDNIIFSEEYNTSNPDLLL
VIFQSKYGPPCPPCPAPEFLGGPSVFLFPPKPKDQ
LMISRTPEVTCVVDVSQEDPEVQFNWYVDGVEVHNA
KTKPREEQFNSTYRVVSVLTVLHQDWLSGKEYKCK
VSSKGLPSSIEKTISNATGQPREPQVYTLPPSQEE
MTKNQVSLTCLVKGFYPSDIAVEWESNGQPENNYK
TTPPVLDSDGSFFLYSRLTVDKSRWQEGNVFSCSV
LHEALHNHYTQKSLSLSLGKIEGRMDQRTDSIPNS
PDNVPLKGGNCSEDLLCILKRAPFKKSWAYLQVAK
HLNKTKLSWNKDGILHGVRYQDGNLVIQFPGLYFI
ICQLQFLVQCPNNSVDLKLELLINKHIKKQALVTV
CESGMQTKHVYQNLSQFLLDYLQVNTTISVNVDTF
QYIDTSTFPLENVLSIFLYSNSD

In embodiments, the chimeric protein of the present invention comprises SEQ ID NO: 10, i.e., monomeric TGFBR2-Fc-CD30L chimeric protein, or a variant or functional fragment thereof.

In embodiments, the chimeric protein may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 10.

In embodiments, the chimeric protein of the present invention comprises an extracellular domain of TGFBR2 and the extracellular domain of 4-1BBL, using the hinge-CH2-CH3 domain from a human IgG4 antibody sequence as a linker (this TGFBR2-Fc-NKG2A chimera is SEQ ID NO: 11):

(SEQ ID NO: 11)
TIPPHVQKSVNNDMIVTDNNGAVKFPQLCKFCDVR
FSTCDNQKSCMSNCSITSICEKPQEVCVAVWRKND
ENITLETVCHDPKLPYHDFILEDAASPKCIMKEKK
KPGETFFMCSCSSDECNDNIIFSEEYNTSNPDLLL
VIFQSKYGPPCPPCPAPEFLGGPSVFLFPPKPKDQ
LMISRTPEVTCVVDVSQEDPEVQFNWYVDGVEVHNA
KTKPREEQFNSTYRVVSVLTVLHQDWLSGKEYKCK
VSSKGLPSSIEKTISNATGQPREPQVYTLPPSQEE
MTKNQVSLTCLVKGFYPSDIAVEWESNGQPENNYK
TTPPVLDSDGSFFLYSRLTVDKSRWQEGNVFSCSV
LHEALHNHYTQKSLSLSLGKIEGRMDPSTLIQRHN
NSSLNTRTQKARHCGHCPEEWITYSNSCYYIGKER
RTWEESLLACTSKNSSLLSIDNEEEMKFLSIISPS
SWIGVFRNSSHHPWVTMNGLAFKHEIKDSDNAELN
CAVLQVNRLKSAQCGSSIIYHCKHKL

In embodiments, the chimeric protein of the present invention comprises SEQ ID NO: 11, i.e., monomeric TGFBR2-Fc-NKG2A chimeric protein, or a variant or functional fragment thereof.

In embodiments, the chimeric protein may have at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of any one of SEQ ID NO: 11.

In embodiments, the chimeric proteins of the invention may comprise a sequence which has one or more amino acid mutations with respect to any one of the sequences disclosed herein. In embodiments, the chimeric protein comprises a sequence that has about 1, 2, 3, 4, 5, 6, 7, 8, 9, 10, 15, 20, 25, 30, 35, 40, 45, 50, 55, 60, 65, 70, 75, 80, 85, 90, 95, or 100 or more amino acid mutations with respect to any one of the amino acid sequences of chimeric proteins disclosed herein.

In embodiments, the one or more amino acid mutations may be independently selected from substitutions, insertions, deletions, and truncations.

In embodiments, the amino acid mutations are amino acid substitutions, and may include conservative and/or non-conservative substitutions.

“Conservative substitutions” may be made, for instance, on the basis of similarity in polarity, charge, size, solubility, hydrophobicity, hydrophilicity, and/or the amphipathic nature of the amino acid residues involved. The 20 naturally occurring amino acids can be grouped into the following six standard amino acid groups: (1) hydrophobic: Met, Ala, Val, Leu, Ile; (2) neutral hydrophilic: Cys, Ser, Thr; Asn, Gln; (3) acidic: Asp, Glu; (4) basic: His, Lys, Arg; (5) residues that influence chain orientation: Gly, Pro; and (6) aromatic: Trp, Tyr, Phe.

As used herein, “conservative substitutions” are defined as exchanges of an amino acid with another amino acid listed within the same group of the six standard amino acid groups shown above. For example, the exchange of Asp by Glu retains one negative charge in the so-modified polypeptide. In addition, glycine and proline may be substituted for one another based on their ability to disrupt α-helices.

As used herein, “non-conservative substitutions” are defined as exchanges of an amino acid with another amino acid listed in a different group of the six standard amino acid groups (1) to (6) shown above.

In embodiments, the substitutions may also include non-classical amino acids (e.g., selenocysteine, pyrrolysine, N-formylmethionine β-alanine, GABA and δ-Aminolevulinic acid, 4-aminobenzoic acid (PABA), D-isomers of the common amino acids, 2,4-diaminobutyric acid, α-amino isobutyric acid, 4-aminobutyric acid, Abu, 2-amino butyric acid, γ-Abu, ε-Ahx, 6-amino hexanoic acid, Aib, 2-amino isobutyric acid, 3-amino propionic acid, ornithine, norleucine, norvaline, hydroxyproline, sarcosme, citrulline, homocitrulline, cysteic acid, t-butylglycine, t-butylalanine, phenylglycine, cyclohexylalanine, β-alanine, fluoro-amino acids, designer amino acids such as β methyl amino acids, C α-methyl amino acids, N α-methyl amino acids, and amino acid analogs in general).

Mutations may also be made to the nucleotide sequences of the chimeric proteins by reference to the genetic code, including taking into account codon degeneracy.

In embodiments, the chimeric protein comprises a linker. In embodiments, the linker comprising at least one cysteine residue capable of forming a disulfide bond. As described elsewhere herein, such at least one cysteine residue capable of forming a disulfide bond is, without wishing to be bound by theory, responsible for maintain a proper multimeric state of the chimeric protein and allowing for efficient production.

In embodiments, the present invention provides a recombinant fusion protein comprising a general structure of: N terminus-(a)-(b)-(c)-C terminus, where (a) is a first domain comprising an extracellular domain of TGFBR2 that is at least 95% identical to the amino acid sequence of ______ and is capable of binding a TGFBR2 ligand, (b) is a linker linking the first domain and the second domain and comprising a hinge-CH2-CH3 Fc domain derived from human IgG4 (e.g. 95% identical to the amino acid sequence of SEQ ID NO: 25, SEQ ID NO: 26, or SEQ ID NO: 27, and (c) is a second domain comprising an extracellular domain of 4-1BB Ligand (4-1BBL), that is at least 95% identical to the amino acid sequence of SEQ ID NO: 4 and is capable of binding an 4-1BB. In embodiments, the present invention provides methods of treating cancer with this heterologous chimeric protein. In embodiments, the present invention provides methods of treating an inflammatory disease with this heterologous chimeric protein.

In embodiments, the present invention provides a recombinant fusion protein comprising a general structure of: N terminus-(a)-(b)-(c)-C terminus, where (a) is a first domain comprising an extracellular domain of TGFBR2 that is at least 95% identical to the amino acid sequence of ______ and is capable of binding a TGFBR2 ligand, (b) is a linker linking the first domain and the second domain and comprising a hinge-CH2-CH3 Fc domain derived from human IgG4 (e.g. 95% identical to the amino acid sequence of SEQ ID NO: 25, SEQ ID NO: 26, or SEQ ID NO: 27, and (c) is a second domain comprising an extracellular domain of CD30 Ligand (CD30L), that is at least 95% identical to the amino acid sequence of SEQ ID NO: 6 and is capable of binding an CD30. In embodiments, the present invention provides methods of treating cancer with this heterologous chimeric protein. In embodiments, the present invention provides methods of treating an inflammatory disease with this heterologous chimeric protein.

In embodiments, the present invention provides a recombinant fusion protein comprising a general structure of: N terminus-(a)-(b)-(c)-C terminus, where (a) is a first domain comprising an extracellular domain of TGFBR2 that is at least 95% identical to the amino acid sequence of ______ and is capable of binding a TGFBR2 ligand, (b) is a linker linking the first domain and the second domain and comprising a hinge-CH2-CH3 Fc domain derived from human IgG4 (e.g. 95% identical to the amino acid sequence of SEQ ID NO: 25, SEQ ID NO: 26, or SEQ ID NO: 27, and (c) is a second domain comprising an extracellular domain of NKG2A, that is at least 95% identical to the amino acid sequence of SEQ ID NO: 8 and is capable of binding an HLA-E. In embodiments, the present invention provides methods of treating cancer with this heterologous chimeric protein. In embodiments, the present invention provides methods of treating an inflammatory disease with this heterologous chimeric protein.

In embodiments, chimeric protein is a recombinant fusion protein, e.g., a single polypeptide having the extracellular domains described herein (and, optionally a linker). For example, in embodiments, the chimeric protein is translated as a single unit in a cell. In embodiments, a chimeric protein refers to a recombinant protein of multiple polypeptides, e.g. multiple extracellular domains described herein, that are linked to yield a single unit, e.g. in vitro (e.g. with one or more synthetic linkers described herein). In embodiments, the chimeric protein is chemically synthesized as one polypeptide or each domain may be chemically synthesized separately and then combined. In embodiments, a portion of the chimeric protein is translated and a portion is chemically synthesized.

In embodiments, the present chimeric proteins may be variants described herein, for instance, the present chimeric proteins may have a sequence having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99%) sequence identity with the amino acid sequence of the present chimeric proteins, e.g. one or more of SEQ IDs Nos 5 and 8.

In embodiments, the chimeric protein comprises a linker. In embodiments, the linker may be derived from naturally-occurring multi-domain proteins or are empirical linkers as described, for example, in Chichili et al., (2013), Protein Sci. 22(2):153-167, Chen et al., (2013), Adv Drug Deliv Rev. 65(10):1357-1369, the entire contents of which are hereby incorporated by reference. In embodiments, the linker may be designed using linker designing databases and computer programs such as those described in Chen et al., (2013), Adv Drug Deliv Rev. 65(10):1357-1369 and Crasto et. al., (2000), Protein Eng. 13(5):309-312, the entire contents of which are hereby incorporated by reference.

In embodiments, the linker is a synthetic linker such as PEG.

In embodiments, the linker comprises a polypeptide. In embodiments, the polypeptide is less than about 500 amino acids long, about 450 amino acids long, about 400 amino acids long, about 350 amino acids long, about 300 amino acids long, about 250 amino acids long, about 200 amino acids long, about 150 amino acids long, or about 100 amino acids long. For example, the linker may be less than about 100, about 95, about 90, about 85, about 80, about 75, about 70, about 65, about 60, about 55, about 50, about 45, about 40, about 35, about 30, about 25, about 20, about 19, about 18, about 17, about 16, about 15, about 14, about 13, about 12, about 11, about 10, about 9, about 8, about 7, about 6, about 5, about 4, about 3, or about 2 amino acids long. In embodiments, the linker is flexible. In an embodiment, the linker is rigid.

In embodiments, the linker is substantially comprised of glycine and serine residues (e.g., about 30%, or about 40%, or about 50%, or about 60%, or about 70%, or about 80%, or about 90%, or about 95%, or about 97%, or about 98%, or about 99%, or about 100% glycines and serines).

In embodiments, the linker comprises a hinge region of an antibody (e.g., of IgG, IgA, IgD, and IgE, inclusive of subclasses (e.g., IgG1, IgG2, IgG3, and IgG4, and IgA1 and IgA2)). The hinge region, found in IgG, IgA, IgD, and IgE class antibodies, acts as a flexible spacer, allowing the Fab portion to move freely in space. In contrast to the constant regions, the hinge domains are structurally diverse, varying in both sequence and length among immunoglobulin classes and subclasses. For example, the length and flexibility of the hinge region varies among the IgG subclasses. The hinge region of IgG1 encompasses amino acids 216-231 and, because it is freely flexible, the Fab fragments can rotate about their axes of symmetry and move within a sphere centered at the first of two inter-heavy chain disulfide bridges. IgG2 has a shorter hinge than IgG1, with 12 amino acid residues and four disulfide bridges. The hinge region of IgG2 lacks a glycine residue, is relatively short, and contains a rigid poly-proline double helix, stabilized by extra inter-heavy chain disulfide bridges. These properties restrict the flexibility of the IgG2 molecule. IgG3 differs from the other subclasses by its unique extended hinge region (about four times as long as the IgG1 hinge), containing 62 amino acids (including 21 prolines and 11 cysteines), forming an inflexible poly-proline double helix. In IgG3, the Fab fragments are relatively far away from the Fc fragment, giving the molecule a greater flexibility. The elongated hinge in IgG3 is also responsible for its higher molecular weight compared to the other subclasses. The hinge region of IgG4 is shorter than that of IgG1 and its flexibility is intermediate between that of IgG1 and IgG2. The flexibility of the hinge regions reportedly decreases in the order IgG3>IgG1>IgG4>IgG2. In embodiments, the linker may be derived from human IgG4 and contain one or more mutations to enhance dimerization (including S228P) or FcRn binding.

According to crystallographic studies, the immunoglobulin hinge region can be further subdivided functionally into three regions: the upper hinge region, the core region, and the lower hinge region. See Shin et al., 1992 Immunological Reviews 130:87. The upper hinge region includes amino acids from the carboxyl end of C_{H1} to the first residue in the hinge that restricts motion, generally the first cysteine residue that forms an interchain disulfide bond between the two heavy chains. The length of the upper hinge region correlates with the segmental flexibility of the antibody. The core hinge region contains the inter-heavy chain disulfide bridges, and the lower hinge region joins the amino terminal end of the C_{H2} domain and includes residues in C_{H2}. Id. The core hinge region of wild-type human IgG1 contains the sequence CPPC (SEQ ID NO: 48) which, when dimerized by disulfide bond formation, results in a cyclic octapeptide believed to act as a pivot, thus conferring flexibility. In embodiments, the present linker comprises, one, or two, or three of the upper hinge region, the core region, and the lower hinge region of any antibody (e.g., of IgG, IgA, IgD, and IgE, inclusive of subclasses (e.g., IgG1, IgG2, IgG3, and IgG4, and IgA1 and IgA2)). The hinge region may also contain one or more glycosylation sites, which include a number of structurally distinct types of sites for carbohydrate attachment. For example, IgA1 contains five glycosylation sites within a 17-amino-acid segment of the hinge region, conferring resistance of the hinge region polypeptide to intestinal proteases, considered an advantageous property for a secretory immunoglobulin. In embodiments, the linker of the present invention comprises one or more glycosylation sites.

In embodiments, the linker comprises an Fc domain of an antibody (e.g., of IgG, IgA, IgD, and IgE, inclusive of subclasses (e.g., IgG1, IgG2, IgG3, and IgG4, and IgA1 and IgA2)). In embodiments, the linker comprises a hinge-CH2-CH3 Fc domain derived from a human IgG4 antibody. In embodiments, the linker comprises a hinge-CH2-CH3 Fc domain derived from a human IgG1 antibody. In embodiments, the Fc domain exhibits increased affinity for and enhanced binding to the neonatal Fc receptor (FcRn). In embodiments, the Fc domain includes one or more mutations that increases the affinity and enhances binding to FcRn. Without wishing to be bound by theory, it is believed that increased affinity and enhanced binding to FcRn increases the in vivo half-life of the present chimeric proteins.

In embodiments, the Fc domain in a linker contains one or more amino acid substitutions at amino acid residue 250, 252, 254, 256, 308, 309, 311, 416, 428, 433 or 434 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference), or equivalents thereof. In embodiments, the amino acid substitution at amino acid residue 250 is a substitution with glutamine. In embodiments, the amino acid substitution at amino acid residue 252 is a substitution with tyrosine, phenylalanine, tryptophan or threonine. In embodiments, the amino acid substitution at amino acid residue 254 is a substitution with threonine. In embodiments, the amino acid substitution at amino acid residue 256 is a substitution with serine, arginine, glutamine, glutamic acid, aspartic acid, or threonine. In embodiments, the amino acid substitution at amino acid residue 308 is a substitution with threonine. In embodiments, the amino acid substitution at amino acid residue 309 is a substitution with proline. In embodiments, the amino acid substitution at amino acid residue 311 is a substitution with serine. In embodiments, the amino acid substitution at amino acid residue 385 is a substitution with arginine, aspartic acid, serine, threonine, histidine, lysine, alanine or glycine. In embodiments, the amino acid substitution at amino acid residue 386 is a substitution with threonine, proline, aspartic acid, serine, lysine, arginine, isoleucine, or methionine. In embodiments, the amino acid substitution at amino acid residue 387 is a substitution with arginine, proline, histidine, serine, threonine, or alanine. In embodiments, the amino acid substitution at amino acid residue 389 is a substitution with proline, serine or asparagine. In embodiments, the amino acid substitution at amino acid residue 416 is a substitution with serine. In embodiments, the amino acid substitution at amino acid residue 428 is a substitution with leucine. In embodiments, the amino acid substitution at amino acid residue 433 is a substitution with arginine, serine, isoleucine, proline, or glutamine. In embodiments, the amino acid substitution at amino acid residue 434 is a substitution with histidine, phenylalanine, or tyrosine.

In embodiments, the Fc domain in a linker (e.g., comprising an IgG constant region) comprises one or more mutations such as substitutions at amino acid residue 252, 254, 256, 433, 434, or 436 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). In embodiments, the IgG constant region includes a triple M252Y/S254T/T256E mutation or YTE mutation. In an embodiment, the IgG constant region includes a triple H433K/N434F/Y436H mutation or KFH mutation. In embodiments, the IgG constant region includes an YTE and KFH mutation in combination.

In embodiments, the modified humanized antibodies of the invention comprise an IgG constant region that contains one or more mutations at amino acid residues 250, 253, 307, 310, 380, 428, 433, 434, and 435 (in accordance with Kabat numbering, as in as in Kabat, et al., Sequences of Proteins of Immunological Interest, 5th Ed. Public Health Service, National Institutes of Health, Bethesda, Md. (1991) expressly incorporated herein by reference). Illustrative mutations include T250Q, M428L, 1307A, E380A, 1253A, H310A, M428L, H433K, N434A, N434F, N434S, and H435A. In embodiments, the IgG constant region comprises a M428L/N434S mutation or LS mutation. In an embodiment, the IgG constant region comprises a T250Q/M428L mutation or QL mutation. In an embodiment, the IgG constant region comprises an N434A mutation. In an embodiment, the IgG constant region comprises a T307A/E380A/N434A mutation or AAA mutation. In an embodiment, the IgG constant region comprises an 1253A/H310A/H435A mutation or IHH mutation. In an embodiment, the IgG constant region comprises a H433K/N434F mutation. In an embodiment, the IgG constant region comprises a M252Y/S254T/T256E and a H433K/N434F mutation in combination.

Additional exemplary mutations in the IgG constant region are described, for example, in Robbie, et al., Antimicrobial Agents and Chemotherapy (2013), 57(12):6147-6153, Dall'Acqua et al., JBC (2006), 281(33):23514-24, Dall'Acqua et al., Journal of Immunology (2002), 169:5171-80, Ko et al. Nature (2014) 514:642-645, Grevys et al. Journal of Immunology. (2015), 194(11):5497-508, and U.S. Pat. No. 7,083,784, the entire contents of which are hereby incorporated by reference.

In embodiments, the linker is a polypeptide selected from a flexible amino acid sequence, an IgG hinge region, or an antibody sequence. In embodiments, the linker comprises hinge-CH2-CH3 Fc domain derived from IgG4, preferably human IgG4. In embodiments, the linker comprises hinge-CH2-CH3 Fc domain derived from IgG1, preferably human IgG1. In embodiments, the linker comprises an amino acid sequence that is at least 95% identical to the amino acid sequence of SEQ ID NO: 25, SEQ ID NO: 26, or SEQ ID NO: 27. In embodiments, the linker comprises one or more joining linkers, such joining linkers independently selected from SEQ ID NOs: 28-74. In embodiments, the linker comprises two or more joining linkers each joining linker independently selected from SEQ ID NOs: 28-74; wherein one joining linker is N terminal to the hinge-CH2-CH3 Fc domain and another joining linker is C terminal to the hinge-CH2-CH3 Fc domain.

In embodiments, the Fc domain in a linker has the amino acid sequence of SEQ ID NO: 25 (see the below table), or at least 90%, or 93%, or 95%, or 97%, or 98%, or 99% identity thereto. In embodiments, mutations are made to SEQ ID NO: 25 to increase stability and/or half-life. For instance, in embodiments, the Fc domain in a linker comprises the amino acid sequence of SEQ ID NO: 26 (see the below table), or at least 90%, or 93%, or 95%, or 97%, or 98%, or 99% identity thereto. An illustrative Fc stabilizing mutant is S228P. Illustrative Fc half-life extending mutants are T250Q, M428L, V308T, L309P, and Q311S and the present linkers may comprise 1, or 2, or 3, or 4, or 5 of these mutants. For instance, in embodiments, the Fc domain in a linker comprises the amino acid sequence of SEQ ID NO: 27 (see the below table), or at least 90%, or 93%, or 95%, or 97%, or 98%, or 99% identity thereto.

Further, one or more joining linkers may be employed to connect an Fc domain in a linker (e.g., one of SEQ ID NO: 25, SEQ ID NO: 26, SEQ ID NO: 27, or at least 90%, or 93%, or 95%, or 97%, or 98%, or 99% identity thereto) and the extracellular domains. For example, any one of SEQ ID NO: 28, SEQ ID NO: 29, SEQ ID NO: 30, SEQ ID NO: 31, SEQ ID NO: 32, SEQ ID NO: 33, or variants thereof may connect an extracellular domain (ECD) as described herein and an Fc domain in a linker as described herein. Optionally, any one of SEQ ID NOs: 28 to 74, or variants thereof are located between an extracellular domain as described herein and an Fc domain as described herein. In embodiments, a chimeric protein comprises one joining linker preceding an Fc domain and a second joining linker following the Fc domain; thus, a chimeric protein may comprise the following structure:
ECD 1 (e.g., TGFBR2)-Joining Linker 1-Fc Domain-Joining Linker 2-ECD 2 (e.g., 4-1BBL, CD30L or NKG2A).

In embodiments, the first and second joining linkers may be different or they may be the same.

In embodiments, the first and second joining linkers may be selected from the amino acid sequences of SEQ ID NOs: 25 to 74 and are provided in Table 1 below:

TABLE 1
Illustrative linkers (Fc domain linkers
and joining linkers)

| SEQ ID | |
|---|---|
| NO. | Sequence |
| 25 | APEFLGGPSVFLFPPKPKDTLMISRTPEVT |
| | CVVVDVSQEDPEVQFNWYVDGVEVHNAKTK |
| | PREEQFNSTYRVVSVLTVLHQDWLSGKEYK |
| | CKVSSKGLPSSIEKTISNATGQPREPQVYT |
| | LPPSQEEMTKNQVSLTCLVKGFYPSDIAVE |
| | WESNGQPENNYKTTPPVLDSDGSFFLYS |
| | RLTVDKSSWQEGNVFSCSVMHEALHNHYTQ |
| | KSLSLSLGK |
| 26 | APEFLGGPSVFLFPPKPKDQLMISRTPEVT |
| | CVVVDVSQEDPEVQFNWYVDGVEVHNAKTK |
| | PREEQFNSTYRVVSVLTTPHSDWLSGKEYK |
| | CKVSSKGLPSSIEKTISNATGQPREPQVYT |
| | LPPSQEEMTKNQVSLTCLVKGFYPSDIAVE |
| | WESNGQPENNYKTTPPVLDSDGSFFLYSRL |
| | TVDKSSWQEGNVFSCSVLHEALHNHYTQKS |
| | LSLSLGK |
| 27 | APEFLGGPSVFLFPPKPKDQLMISRTPEVT |
| | CVVVDVSQEDPEVQFNWYVDGVEVHNAKTK |
| | PREEQFNSTYRVVSVLTVLHQDWLSGKEYK |
| | CKVSSKGLPSSIEKTISNATGQPREPQVYT |
| | LPPSQEEMTKNQVSLTCLVKGFYPSDIAVE |
| | WESNGQPENNYKTTPPVLDSDGSFFLYSRL |
| | TVDKSRWQEGNVFSCSVLHEALHNHYTQKS |
| | LSLSLGK |
| 28 | SKYGPPCPSCP |
| 29 | SKYGPPCPPCP |
| 30 | SKYGPP |
| 31 | IEGRMD |
| 32 | GGGVPRDCG |
| 33 | IEGRMDGGGGAGGGG |
| 34 | GGGSGGGS |
| 35 | GGGSGGGGSGGG |
| 36 | EGKSSGSGSESKST |
| 37 | GGSG |
| 38 | GGSGGGSGGGSG |
| 39 | EAAAKEAAAKEAAAK |
| 40 | EAAAREAAAREAAAREAAAR |
| 41 | GGGGSGGGGSGGGGSAS |
| 42 | GGGGAGGGG |
| 43 | GS or GGS or LE |
| 44 | GSGSGS |
| 45 | GSGSGSGSGS |
| 46 | GGGGSAS |
| 47 | APAPAPAPAPAPAPAPAPAP |
| 48 | CPPC |
| 49 | GGGGS |
| 50 | GGGGSGGGGS |
| 51 | GGGGSGGGGSGGGGS |
| 52 | GGGGSGGGGSGGGGSGGGGS |
| 53 | GGGGSGGGGSGGGGSGGGGSGGGGS |
| 54 | GGGGSGGGGSGGGGSGGGGSGGGGSGGGGS |
| 55 | GGGGSGGGGSGGGGSGGGGSGGGGSGGGGSGGGGS |
| 56 | GGGGSGGGGSGGGGSGGGGSGGGGSGGGGSGGGGS |
| | GGGGS |
| 57 | GGSGGSGGGGSGGGGS |
| 58 | GGGGGGGG |
| 59 | GGGGGG |
| 60 | EAAAK |
| 61 | EAAAKEAAAK |
| 62 | EAAAKEAAAKEAAAK |
| 63 | AEAAAKEAAAKA |
| 64 | AEAAAKEAAAKEAAAKA |
| 65 | AEAAAKEAAAKEAAAKEAAAKA |
| 66 | AEAAAKEAAAKEAAAKEAAAKEAAAKA |
| 67 | AEAAAKEAAAKEAAAKEAAAKALEAEAA |
| | AKEAAAKEAAAKEAAAKA |
| 68 | PAPAP |
| 69 | KESGSVSSEQLAQFRSLD |
| 70 | GSAGSAAGSGEF |
| 71 | GGGSE |
| 72 | GSESG |
| 73 | GSEGS |
| 74 | GEGGSGEGSSGEGSSSEGGGSEGGGSEGGGSEGGS |

In embodiments, the joining linker substantially comprises glycine and serine residues (e.g., about 30%, or about 40%, or about 50%, or about 60%, or about 70%, or about 80%, or about 90%, or about 95%, or about 97%, or about 98%, or about 99%, or about 100% glycines and serines). For example, in embodiments, the joining linker is (Gly₄Ser)ₙ, where n is from about 1 to about 8, e.g., 1, 2, 3, 4, 5, 6, 7, or 8 (SEQ ID NO: 49 to SEQ ID NO: 56, respectively). In embodiments, the joining linker sequence is GGSGGSGGGGSGGGGS (SEQ ID NO: 57). Additional illustrative joining linkers include, but are not limited to, linkers having the sequence LE, (Gly)₈ (SEQ ID NO: 58), (Gly)₆ (SEQ ID NO: 59), (EAAAK)ₙ (n=1-3) (SEQ ID NO: 60-SEQ ID NO: 62), A(EAAAK)ₙA (n=2-5) (SEQ ID NO: 63-SEQ ID NO: 66), A(EAAAK)₄ALEA(EAAAK)₄A (SEQ ID NO: 67), PAPAP (SEQ ID NO: 68), KESGSVSSEQLAQFRSLD (SEQ ID NO: 69), GSAGSAAGSGEF (SEQ ID NO: 70), and (XP)ₙ, with X designating any amino acid, e.g., Ala, Lys, or Glu. In embodiments, the joining linker is GGS.

In embodiments, the joining linker is one or more of GGGSE (SEQ ID NO: 71), GSESG (SEQ ID NO: 72), GSEGS (SEQ ID NO: 73), GEGGSGEGSSGEGSSSEGGGSEGGGSEGGGSEGGS (SEQ ID NO: 74), and a joining linker of randomly placed G, S, and E every 4 amino acid intervals.

In embodiments, the linker may be functional. For example, without limitation, the linker may function to improve the folding and/or stability, improve the expression, improve the pharmacokinetics, and/or improve the bioactivity of the present chimeric protein. In another example, the linker may function to target the chimeric protein to a particular cell type or location.

In embodiments, the chimeric protein exhibits enhanced stability and protein half-life. In embodiments, the chimeric protein binds to FcRn with high affinity. In embodiments, the chimeric protein may bind to FcRn with a K_{D} of about 1 nM to about 80 nM. For example, the chimeric protein may bind to FcRn with a K_{D} of about 1 nM, about 2 nM, about 3 nM, about 4 nM, about 5 nM, about 6 nM, about 7 nM, about 8 nM, about 9 nM, about 10 nM, about 15 nM, about 20 nM, about 25 nM, about 30 nM, about 35 nM, about 40 nM, about 45 nM, about 50 nM, about 55 nM, about 60 nM, about 65 nM, about 70 nM, about 71 nM, about 72 nM, about 73 nM, about 74 nM, about 75 nM, about 76 nM, about 77 nM, about 78 nM, about 79 nM, or about 80 nM. In embodiments, the chimeric protein may bind to FcRn with a K_{D} of about 9 nM. In embodiments, the chimeric protein does not substantially bind to other Fc receptors (i.e., other than FcRn) with effector function.

In embodiments, a chimeric protein having the formula ECD 1-Joining Linker 1-Fc Domain-Joining Linker 2-ECD 2, in which ECD 1 is TGFBR2 and ECD 2 is 4-1BBL may be referred to in the present disclosure as TGFBR2-Fc-4-1BBL. In embodiments, the chimeric protein lacks one or both joining linkers; such a chimeric protein may also be referred to in the present disclosure as TGFBR2-Fc-4-1BBL.

In embodiments, a chimeric protein is a fusion protein having the formula N terminus-(a)-(b)-(c)-C terminus, in which (a) is TGFBR2, (b) is a linker comprising at least a portion of a Fc domain, and (c) is 4-1BBL may be referred to in the present disclosure as TGFBR2-Fc-4-1BBL.

In embodiments, a chimeric protein is optimized for/directed to murine ligands/receptors; an example of such a chimeric protein is murine TGFBR2-Fc-4-1BBL, which is also referred herein as mTGFBR2-Fc-4-1BBL.

In embodiments, a chimeric protein is optimized for/directed to human ligands/receptors; an example of such a chimeric protein is human TGFBR2-Fc-4-1BBL, which is also referred herein as hTGFBR2-Fc-4-1BBL.

In embodiments, a chimeric protein having the formula ECD 1-Joining Linker 1-Fc Domain-Joining Linker 2-ECD 2, in which ECD 1 is TGFBR2 and ECD 2 is CD30L may be referred to in the present disclosure as TGFBR2-Fc-CD30L. In embodiments, the chimeric protein lacks one or both joining linkers; such a chimeric protein may also be referred to in the present disclosure as TGFBR2-Fc-CD30L.

In embodiments, a chimeric protein is a fusion protein having the formula N terminus-(a)-(b)-(c)-C terminus, in which (a) is TGFBR2, (b) is a linker comprising at least a portion of a Fc domain, and (c) is CD30L may be referred to in the present disclosure as TGFBR2-Fc-CD30L.

In embodiments, a chimeric protein is optimized for/directed to murine ligands/receptors; an example of such a chimeric protein is murine TGFBR2-Fc-CD30L, which is also referred herein as mTGFBR2-Fc-CD30L.

In embodiments, a chimeric protein is optimized for/directed to human ligands/receptors; an example of such a chimeric protein is human TGFBR2-Fc-CD30L, which is also referred herein as hTGFBR2-Fc-CD30L.

In embodiments, a chimeric protein having the formula ECD 1-Joining Linker 1-Fc Domain-Joining Linker 2-ECD 2, in which ECD 1 is TGFBR2 and ECD 2 is NKG2A may be referred to in the present disclosure as TGFBR2-Fc-NKG2A. In embodiments, the chimeric protein lacks one or both joining linkers; such a chimeric protein may also be referred to in the present disclosure as TGFBR2-Fc-NKG2A.

In embodiments, a chimeric protein is a fusion protein having the formula N terminus-(a)-(b)-(c)-C terminus, in which (a) is TGFBR2, (b) is a linker comprising at least a portion of a Fc domain, and (c) is NKG2A may be referred to in the present disclosure as TGFBR2-Fc-NKG2A.

In embodiments, a chimeric protein is optimized for/directed to murine ligands/receptors; an example of such a chimeric protein is murine TGFBR2-Fc-NKG2A, which is also referred herein as mTGFBR2-Fc-NKG2A.

In embodiments, a chimeric protein is optimized for/directed to human ligands/receptors; an example of such a chimeric protein is human TGFBR2-Fc-NKG2A, which is also referred herein as hTGFBR2-Fc-NKG2A.

These chimeric proteins may lack one or both of the joining linkers. Exemplary Joining Linker 1s, Fc Domains, and Joining Linker 2s are described above in Table 1. In embodiments, the present chimeric protein is engineered to target the TGFBR2/TGFβ immune inhibitory signaling pathway. In embodiment, the chimeric protein is engineered to disrupt, block, reduce, and/or inhibit the transmission of an immune inhibitory signal mediated by binding of TGFβ to TGFBR2. In embodiments, an immune inhibitory signal refers to a signal that diminishes or eliminates an immune response. For example, in the context of oncology, such signals may diminish or eliminate antitumor immunity. Under normal physiological conditions, inhibitory signals are useful in the maintenance of self-tolerance (e.g., prevention of autoimmunity) and also to protect tissues from damage when the immune system is responding to pathogenic infection. For instance, without limitation, an immune inhibitory signal may be identified by detecting an increase in cellular proliferation, cytokine production, cell killing activity or phagocytic activity when such an inhibitory signal is blocked.

In embodiments, the present chimeric protein disrupts, blocks, reduces, and/or inhibits the transmission of an immune inhibitory signal mediated by the binding of TGFβ (e.g. TGFβ 1 and/or TGFβ3) to TGFBR2. In embodiments, the chimeric protein binds to and sequesters TGFβ (e.g. TGFβ 1 and/or TGFβ3), and thereby disrupts, blocks, reduces, and/or inhibits the inhibitory signal transmission to an immune cell (e.g., a tumor-associated macrophage, antigen presenting cell, myeloid cell, or a T cell).

In embodiments, the present chimeric proteins are capable of, or find use in methods comprising, inhibiting or reducing the binding of the immune inhibitory receptor/ligand pair: TGFBR2/TGFβ or TGFBR2/TGFβ (E.G. TGFB 1 AND/OR TGFB3). In embodiments, the present chimeric protein blocks, reduces, and/or inhibits TGFBR2 activation, for example, by reducing the binding of TGFBR2 on immune cells with TGFβ (e.g. TGFβ 1 and/or TGFβ3).

In embodiments, the present chimeric protein targets an immune stimulatory signal mediated by the binding of 4-1BBL to 4-1BB. In embodiment, the chimeric protein is engineered to enhance, increase, and/or stimulate the transmission of an immune stimulatory signal mediated by binding of 4-1BBL to 4-1BB. In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity or phagocytic activity of leukocytes, including subsets of T cells.

In embodiments, the present chimeric protein enhances, increases, and/or stimulates the transmission of an immune stimulatory signal mediated by the binding of 4-1BBL to 4-1BB. In embodiments, the present chimeric protein comprising the extracellular domain of 4-1BBL acts on an immune cell (e.g., a dendritic cell, a B cell, a macrophage, an antigen presenting cell, or a T cell) that expresses 4-1BB and enhances, increases, and/or stimulates stimulatory signal transmission to the immune cell (e.g., a dendritic cell, a B cell, a macrophage, and a T cell).

In embodiments, the present chimeric proteins are capable of, or find use in methods comprising, stimulating or enhancing the binding of the immune stimulatory receptor/ligand pair: 4-1BB:4-1BBL. In embodiments, the present chimeric protein increases and/or stimulates 4-1BB and/or the binding of 4-1BB with 4-1BBL.

In embodiments, the present chimeric protein targets an immune stimulatory signal mediated by the binding of CD30L to CD30. In embodiment, the chimeric protein is engineered to enhance, increase, and/or stimulate the transmission of an immune stimulatory signal mediated by binding of CD30L to CD30. In embodiments, an immune stimulatory signal refers to a signal that enhances an immune response. For example, in the context of oncology, such signals may enhance antitumor immunity. For instance, without limitation, immune stimulatory signal may be identified by directly stimulating proliferation, cytokine production, killing activity or phagocytic activity of leukocytes, including subsets of T cells.

In embodiments, the present chimeric protein enhances, increases, and/or stimulates the transmission of an immune stimulatory signal mediated by the binding of CD30L to CD30. In embodiments, the present chimeric protein comprising the extracellular domain of CD30L acts on an immune cell (e.g., a dendritic cell, a B cell, a macrophage, an antigen presenting cell, or a T cell) that expresses CD30 and enhances, increases, and/or stimulates stimulatory signal transmission to the immune cell (e.g., a dendritic cell, a B cell, a macrophage, and a T cell).

In embodiments, the present chimeric proteins are capable of, or find use in methods comprising, stimulating or enhancing the binding of the immune stimulatory receptor/ligand pair: CD30:CD30L. In embodiments, the present chimeric protein increases and/or stimulates CD30 and/or the binding of CD30 with CD30L.

In embodiments, the present chimeric protein targets an immune inhibitory signal mediated by the binding of NKG2A to HLA-E. In embodiment, the chimeric protein is engineered to decrease, and/or inhibit the transmission of an immune inhibitory signal mediated by binding of NKG2A to HLA-E. In embodiments, an immune inhibitory signal refers to a signal that reduces an immune response. For example, in the context of oncology, such signals may enhance antitumor immunity. For instance, without limitation, the inhibition of the immune inhibitory signal may be identified by directly stimulating proliferation, cytokine production, killing activity or phagocytic activity of leukocytes, including subsets of T cells.

In embodiments, the present chimeric protein decreases, and/or inhibits the transmission of an immune stimulatory signal mediated by the binding of HLA-E to NKG2A. In embodiments, the present chimeric protein comprising the extracellular domain of HLA-E acts on an immune cell (e.g., an NK cell) that expresses NKG2A and enhances, increases, and/or stimulates stimulatory signal transmission to the immune cell (e.g. an NK-cell).

In embodiments, the present chimeric proteins are capable of, or find use in methods comprising, inhibiting or reducing the binding of the immune stimulatory receptor/ligand pair: NKG2A:HLA-E. In embodiments, the present chimeric protein decreases and/or inhibits NKG2A and/or the binding of NKG2A with HLA-E.

In embodiments, a chimeric protein comprises an extracellular domain of type II protein, other than 4-1BBL, CD30L or NKG2A. Exemplary type II proteins include FasL, GITRL, LIGHT, TL1A, and TRAIL. The present invention further includes chimeric proteins and methods using the following chimeric proteins: TGFBR2/4-1BBL, TGFBR2/CD30L, TGFBR2/FasL, TGFBR2/GITRL, TGFBR2/LIGHT, TGFBR2/NKG2A, TGFBR2/TL1A, and TGFBR2/TRAIL. In embodiments, the chimeric protein has a general structure of one of TGFBR2-Fc-4-1BBL, TGFBR2-Fc-CD30L, TGFBR2-Fc-FasL, TGFBR2-Fc-GITRL, TGFBR2-Fc-LIGHT, TGFBR2-Fc-NKG2A, TGFBR2-Fc-TL1A, and TGFBR2-Fc-TRAIL.

The amino acid sequence for 4-1BBL, CD30L, NKG2A, FasL, GITRL, LIGHT, TL1A, and TRAIL, respectively, comprises SEQ ID NO: 3, 5, 7, 13, 15, 17, 21, and 23.

In embodiments, a chimeric protein comprises the extracellular domain of one of 4-1BBL, CD30L, NKG2A, FasL, GITRL, LIGHT, TL1A, and TRAIL which, respectively, comprises SEQ ID NO: 4, 6, 8, 14, 16, 18, 22, and 24. In embodiments, the present chimeric proteins may comprise the extracellular domain of 4-1BBL, CD30L, FasL, GITRL, LIGHT, NKG2A, TL1A, or TRAIL as described herein, or a variant or a functional fragment thereof. For instance, the chimeric protein may comprise a sequence of the extracellular domain of 4-1BBL, CD30L, FasL, GITRL, LIGHT, NKG2A, TL1A, or TRAIL as provided above, or a variant or functional fragment thereof having at least about 60%, or at least about 61%, or at least about 62%, or at least about 63%, or at least about 64%, or at least about 65%, or at least about 66%, or at least about 67%, or at least about 68%, or at least about 69%, or at least about 70%, or at least about 71%, or at least about 72%, or at least about 73%, or at least about 74%, or at least about 75%, or at least about 76%, or at least about 77%, or at least about 78%, or at least about 79%, or at least about 80%, or at least about 81%, or at least about 82%, or at least about 83%, or at least about 84%, or at least about 85%, or at least about 86%, or at least about 87%, or at least about 88%, or at least about 89%, or at least about 90%, or at least about 91%, or at least about 92%, or at least about 93%, or at least about 94%, or at least about 95%, or at least about 96%, or at least about 97%, or at least about 98%, or at least about 99% sequence identity with the amino acid sequence of the extracellular domain of 4-1BBL, CD30L, FasL, GITRL, LIGHT, NKG2A, TL1A, or TRAIL as described herein.

In embodiments, the chimeric protein of the invention delivers an immune stimulation to an immune cell (e.g., an antigen presenting cell) while providing a localized trap or sequester of immune inhibitory signals. In embodiments, the chimeric protein delivers signals that have the net result of immune activation.

In embodiments, the present chimeric proteins are capable of, and can be used in methods comprising, promoting immune activation (e.g., against tumors). In embodiments, the present chimeric proteins are capable of, and can be used in methods comprising, suppressing immune inhibition (e.g., that allows tumors to survive). In embodiments, the present chimeric proteins provide improved immune activation and/or improved suppression of immune inhibition due to the proximity of signaling that is provided by the chimeric nature of the constructs.

In embodiments, the present chimeric proteins are capable of, or can be used in methods comprising, modulating the amplitude of an immune response, e.g., modulating the level of effector output. In embodiments, e.g., when used for the treatment of a cancer and/or an inflammatory disease, the present chimeric proteins alter the extent of immune stimulation as compared to immune inhibition to increase the amplitude of a T cell response, including, without limitation, stimulating increased levels of cytokine production, proliferation or target killing potential.

In embodiments, the present chimeric proteins are capable of, or find use in methods involving, masking an inhibitory ligand on the surface of a tumor cell and replacing that immune inhibitory ligand with an immune stimulatory ligand. For example, the present chimeric protein comprises (a) an extracellular domain of TGFBR2 and (b) an extracellular domain of 4-1BBL, allows for the disruption of an inhibitory TGFβ/TGFBR2 signal and replacing it with a stimulating 4-1BBL/4-1BB signal. In embodiments, the present chimeric proteins are capable of, or find use in methods involving, masking an inhibitory ligand on the surface of a tumor cell and replacing that immune inhibitory ligand with an immune stimulatory ligand. For example, the present chimeric protein comprises (a) an extracellular domain of TGFBR2 and (b) an extracellular domain of CD30L, allows for the disruption of an inhibitory TGFβ/TGFBR2 signal and replacing it with a stimulating CD30L/CD30 signal. In embodiments, the present chimeric proteins are capable of, or find use in methods involving, masking an inhibitory ligand on the surface of a tumor cell and replacing that immune inhibitory ligand with an immune stimulatory ligand. For example, the present chimeric protein comprises (a) an extracellular domain of TGFBR2 and (b) an extracellular domain of NKG2A, allows for the disruption of an inhibitory TGFβ/TGFBR2 signal and replacing it with a stimulating NKG2A/HLA-E signal. Accordingly, the present chimeric proteins, in embodiments are capable of, or find use in methods involving, reducing or eliminating an inhibitory immune signal and/or increasing or activating an immune stimulatory signal. For example, a tumor comprising an inhibitory signal (and thus evading an immune response) may be substituted for a positive signal binding on a macrophage or a T cell that can then attack a tumor cell. Accordingly, in embodiments, an inhibitory immune signal is masked by the present constructs and a stimulatory immune signal is activated. Such beneficial properties are enhanced by the single construct approach of the present chimeric proteins. For instance, the signal replacement can be effected nearly simultaneously, e.g., contemporaneously, and the signal replacement is tailored to be local at a site of clinical importance (e.g., the tumor microenvironment).

In embodiments, the present chimeric proteins are capable of, or find use in methods involving, enhancing, restoring, promoting and/or stimulating immune modulation. In embodiments, the present chimeric proteins described herein, restore, promote and/or stimulate the activity or activation of one or more immune cells against tumor cells including, but not limited to: T cells, cytotoxic T lymphocytes, T helper cells, natural killer (NK) cells, natural killer T (NKT) cells, anti-tumor macrophages (e.g., M1 macrophages), B cells, and dendritic cells. In embodiments, the present chimeric proteins enhance, restore, promote and/or stimulate the activity and/or activation of T cells, including, by way of a non-limiting example, activating and/or stimulating one or more T-cell intrinsic signals, including a pro-survival signal; an autocrine or paracrine growth signal; a p38 MAPK-, ERK-, STAT-, JAK-, AKT- or PI3K-mediated signal; an anti-apoptotic signal; and/or a signal promoting and/or necessary for one or more of: proinflammatory cytokine production or T cell migration or T cell tumor infiltration.

In embodiments, the present chimeric proteins are capable of, or find use in methods involving, causing an increase of one or more of T cells (including without limitation cytotoxic T lymphocytes, T helper cells, natural killer T (NKT) cells), B cells, natural killer (NK) cells, natural killer T (NKT) cells, dendritic cells, monocytes, and macrophages (e.g., one or more of M1 and M2) into a tumor or the tumor microenvironment. In embodiments, the chimeric protein enhances recognition of tumor antigens by CD8+ T cells, particularly those T cells that have infiltrated into the tumor microenvironment. In embodiments, the present chimeric protein induces CD19 expression and/or increases the number of CD19 positive cells (e.g., CD19 positive B cells). In an embodiment, the present chimeric protein induces IL-15Rα expression and/or increases the number of IL-15Rα positive cells (e.g., IL-15Rα positive dendritic cells).

In embodiments, the present chimeric proteins are capable of, or find use in methods involving, inhibiting and/or causing a decrease in immunosuppressive cells (e.g., myeloid-derived suppressor cells (MDSCs), regulatory T cells (Tregs), tumor associated neutrophils (TANs), M2 macrophages, and tumor associated macrophages (TAMs)), and particularly within the tumor and/or tumor microenvironment (TME). In embodiments, the present therapies may alter the ratio of M1 versus M2 macrophages in the tumor site and/or TME to favor M1 macrophages.

In embodiments, the present chimeric proteins are capable of, and can be used in methods comprising, inhibiting and/or reducing T cell inactivation and/or immune tolerance to a tumor, comprising administering an effective amount of a chimeric protein described herein to a subject. In embodiments, the present chimeric proteins are able to increase the serum levels of various cytokines including, but not limited to, one or more of IFNγ, INFα, IL-2, IL-4, IL-5, IL-6, IL-9, IL-10, IL-13, IL-17A, IL-17F, and IL-22. In embodiments, the present chimeric proteins are capable of enhancing IL-2, IL-4, IL-5, IL-10, IL-13, IL-17A, IL-22, INFα, or IFNγ in the serum of a treated subject. Detection of such a cytokine response may provide a method to determine the optimal dosing regimen for the indicated chimeric protein.

In embodiments, the present chimeric proteins inhibit, block and/or reduce cell death of an anti-tumor CD8+ and/or CD4+ T cell; or stimulate, induce, and/or increase cell death of a pro-tumor T cell. T cell exhaustion is a state of T cell dysfunction characterized by progressive loss of proliferative and effector functions, culminating in clonal deletion. Accordingly, a pro-tumor T cell refers to a state of T cell dysfunction that arises during many chronic infections, inflammatory diseases, and cancer. This dysfunction is defined by poor proliferative and/or effector functions, sustained expression of inhibitory receptors and a transcriptional state distinct from that of functional effector or memory T cells. Exhaustion prevents optimal control of infection and tumors. Illustrative pro-tumor T cells include, but are not limited to, Tregs, CD4+ and/or CD8+ T cells expressing one or more checkpoint inhibitory receptors, Th2 cells and Th17 cells. Checkpoint inhibitory receptors refer to receptors expressed on immune cells that prevent or inhibit uncontrolled immune responses. In contrast, an anti-tumor CD8+ and/or CD4+ T cell refers to T cells that can mount an immune response to a tumor.

In embodiments, the present chimeric proteins are capable of, and can be used in methods comprising, increasing a ratio of effector T cells to regulatory T cells. Illustrative effector T cells include ICOS⁺ effector T cells; cytotoxic T cells (e.g., αβ TCR, CD3⁺, CD8⁺, CD45RO⁺); CD4⁺ effector T cells (e.g., αβ TCR, CD3⁺, CD4⁺, CCR7⁺, CD62Lhi, IL⁻7R/CD127⁺); CD8⁺ effector T cells (e.g., αβ TCR, CD3⁺, CD8⁺, CCR7⁺, CD62Lhi, IL⁻7R/CD127⁺); effector memory T cells (e.g., CD62Llow, CD44⁺, TCR, CD3⁺, IL⁻7R/CD127⁺, IL-15R⁺, CCR7low); central memory T cells (e.g., CCR7⁺, CD62L⁺, CD27⁺; or CCR7hi, CD44⁺, CD62Lhi, TCR, CD3⁺, IL-7R/CD127⁺, IL-15R⁺); CD62L⁺ effector T cells; CD8⁺ effector memory T cells (TEM) including early effector memory T cells (CD27⁺ CD62L⁻) and late effector memory T cells (CD27⁻CD62L⁻) (TemE and TemL, respectively); CD127(⁺)CD25(low/−) effector T cells; CD127(⁻)CD25(⁻) effector T cells; CD8⁺ stem cell memory effector cells (TSCM) (e.g., CD44(low)CD62L(high)CD122(high)sca(⁺)); TH1 effector T-cells (e.g., CXCR3⁺, CXCR6⁺ and CCR5⁺; or αβ TCR, CD3⁺, CD4⁺, IL-12R⁺, IFNγR⁺, CXCR3⁺), TH2 effector T cells (e.g., CCR3⁺, CCR4⁺ and CCR8⁺; or αβ TCR, CD3⁺, CD4⁺, IL-4R⁺, IL-33R⁺, CCR4⁺, IL-17RB⁺, CRTH2⁺); TH9 effector T cells (e.g., αβ TCR, CD3⁺, CD4⁺); TH17 effector T cells (e.g., αβ TCR, CD3⁺, CD4⁺, IL-23R⁺, CCR6⁺, IL-1R⁺); CD4⁺CD45RO⁺CCR7⁺ effector T cells, CD4⁺CD45RO⁺CCR7(⁻) effector T cells; and effector T cells secreting IL-2, IL-4 and/or IFN-γ. Illustrative regulatory T cells include ICOS⁺ regulatory T cells, CD4⁺CD25⁺FOXP3⁺ regulatory T cells, CD4⁺CD25⁺ regulatory T cells, CD4⁺CD25⁻ regulatory T cells, CD4⁺CD25high regulatory T cells, TIM-3⁺PD-1⁺ regulatory T cells, lymphocyte activation gene-3 (LAG-3)⁺ regulatory T cells, CTLA-4/CD152⁺ regulatory T cells, neuropilin-1 (Nrp-1)⁺ regulatory T cells, CCR4⁺CCR8⁺ regulatory T cells, CD62L (L-selectin)⁺ regulatory T cells, CD45RBlow regulatory T cells, CD127low regulatory T cells, LRRC32/GARP⁺ regulatory T cells, CD39⁺ regulatory T cells, GITR⁺ regulatory T cells, LAP⁺ regulatory T cells, 1B11⁺ regulatory T cells, BTLA⁺ regulatory T cells, type 1 regulatory T cells (Tr1 cells), T helper type 3 (Th3) cells, regulatory cell of natural killer T cell phenotype (NKTregs), CD8⁺ regulatory T cells, CD8⁺CD28⁻ regulatory T cells and/or regulatory T-cells secreting IL-10, IL-35, TGF-β, TNF-α, Galectin-1, IFN-γ and/or MCP1.

In embodiments, the chimeric protein of the invention causes an increase in effector T cells (e.g., CD4+CD25− T cells).

In embodiments, the chimeric protein causes a decrease in regulatory T cells (e.g., CD4+CD25+ T cells).

In embodiments, the chimeric protein generates a memory response which may, e.g., be capable of preventing relapse or protecting the animal from a rechallenge. Thus, an animal treated with the chimeric protein is later able to attack tumor cells and/or prevent development of tumors when rechallenged after an initial treatment with the chimeric protein. Accordingly, a chimeric protein of the present invention stimulates both active tumor destruction and also immune recognition of tumor antigens, which are essential in programming a memory response capable of preventing relapse.

In embodiments, the present chimeric proteins are capable of, and can be used in methods comprising, transiently stimulating effector immune cells for no longer than about 12 hours, about 24 hours, about 48 hours, about 72 hours or about 96 hours or about 1 week or about 2 weeks. In embodiments, the present chimeric proteins are capable of, and can be used in methods comprising, transiently depleting or inhibiting regulatory or immune suppressive cells for no longer than about 12 hours, about 24 hours, about 48 hours, about 72 hours or about 96 hours or about 1 week or about 2 weeks. In embodiments, the transient stimulation of effector T cells and/or transient depletion or inhibition of immune inhibitory cells occurs substantially in a patient's bloodstream or in a particular tissue/location including lymphoid tissues such as for example, the bone marrow, lymph-node, spleen, thymus, mucosa-associated lymphoid tissue (MALT), non-lymphoid tissues, or in the tumor microenvironment.

In embodiments, the present chimeric proteins provide advantages including, without limitation, ease of use and ease of production. This is because two distinct immunotherapy agents are combined into a single product which allows for a single manufacturing process instead of two independent manufacturing processes. In addition, administration of a single agent instead of two separate agents allows for easier administration and greater patient compliance.

In embodiments, the present chimeric protein is producible in a mammalian host cell as a secretable and fully functional single polypeptide chain.

In embodiments, the present chimeric protein unexpectedly provides binding of the extracellular domain components to their respective binding partners with slow off rates (Kd or K_{off}). In embodiments, this provides an unexpectedly long interaction of the receptor to ligand and vice versa. Such an effect allows for a sustained negative signal masking effect. Further, in embodiments, this delivers a longer positive signal effect, e.g., to allow an effector cell to be adequately stimulated for an anti-tumor effect. For example, the present chimeric protein, e.g., via the long off rate binding allows sufficient signal transmission to provide immune cell proliferation and allow for anti-tumor attack. By way of further example, the present chimeric protein, e.g., via the long off rate binding allows sufficient signal transmission to provide release of stimulatory signals, such as, for example, cytokines.

The stable synapse of cells promoted by the present agents (e.g. a tumor cell bearing negative signals and a T cell which could attack the tumor) provides spatial orientation to favor tumor reduction—such as positioning the T cells to attack tumor cells and/or sterically preventing the tumor cell from delivering negative signals, including negative signals beyond those masked by the chimeric protein of the invention.

In embodiments, the present chimeric protein exhibits a Kd (1/s) for human TGFβ (e.g. TGFβ 1 and/or TGFβ3) of more than about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶ (as measured, for example, by surface plasmon resonance or biolayer interferometry). In embodiments, the chimeric protein binds to human TGFβ with a K_{D} of from about 100 pM to about 600 pM. In embodiments, the chimeric protein binds to human TGFβ with a Kₐ on rate (1/Ms) of about 5.7×10⁴ and unbinds from human TGFβ with a K_{d} on rate (1/s) of about 7.3×10⁻⁶.

In embodiments, the present chimeric protein exhibits a Kd (1/s) for human 4-1BB of more than about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶ (as measured, for example, by surface plasmon resonance or biolayer interferometry). In embodiments, the chimeric protein binds to human 4-1BB with a Kₐ on rate (1/Ms) of about 1.3×10⁴ and unbinds from human 4-1BB with a Kd off rate (1/s) of about 6.7×10⁻⁶.

In embodiments, the present chimeric protein exhibits a Kd (1/s) for human CD30 of more than about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶ (as measured, for example, by surface plasmon resonance or biolayer interferometry). In embodiments, the chimeric protein binds to human CD30 with a Kₐ on rate (1/Ms) of about 1.3×10⁴ and unbinds from human CD30 with a K_{d} off rate (1/s) of about 6.7×10⁻⁶.

In embodiments, the present chimeric protein exhibits a Kd (1/s) for human NKG2A of more than about 2×10⁶, about 2.5×10⁶, about 3×10⁶, about 3.5×10⁶, about 4×10⁶, about 4.5×10⁶, about 5×10⁶, about 5.5×10⁶, about 6×10⁶, about 6.5×10⁶, about 7×10⁶, about 7.5×10⁶, about 8×10⁶, about 8.5×10⁶, about 9×10⁶, or about 9.5×10⁶ (as measured, for example, by surface plasmon resonance or biolayer interferometry). In embodiments, the chimeric protein binds to human NKG2A with a Kₐ on rate (1/Ms) of about 1.3×10⁴ and unbinds from human NKG2A with a K_{d} off rate (1/s) of about 6.7×10⁻⁶.

In embodiments, this provides longer on-target (e.g., intra-tumoral) half-life (t_{1/2}) as compared to serum t_{1/2} of the chimeric proteins. Such properties could have the combined advantage of reducing off-target toxicities associated with systemic distribution of the chimeric proteins.

In embodiments, the chimeric protein is capable of forming a stable synapse between cells. In embodiments, the stable synapse between cells provides a spatial orientation that favors tumor reduction. In embodiments, the spatial orientation positions T cells to attack tumor cells and/or sterically prevents a tumor cell from delivering negative signals, including negative signals beyond those masked by the chimeric protein of the invention. In embodiments, the stable synapse between cells provides spatial orientation that favors tumor reduction or killing of virus-infected cells by the NK cell. In embodiments, the spatial orientation positions the NK cell to attack target cells selected from tumor cells and virus-infected cells and/or sterically prevents the target cells from delivering negative signals, including negative signals beyond those masked by the chimeric protein of the invention. In embodiments, binding of either or both of the extracellular domains to its respective binding partner occurs with slow off rates (K_{off}), which provides a long interaction of a receptor and its ligand. In embodiments, the long interaction provides sustained negative signal masking effect. In embodiments, the long interaction delivers a longer positive signal effect. In embodiments, the longer positive signal effect allows an effector cell to be adequately stimulated for an anti-tumor effect. In embodiments, the long interaction provides T cell proliferation and allows for anti-tumor attack. In embodiments, the long interaction allows sufficient signal transmission to provide release of stimulatory signals. In embodiments, the stimulatory signal is a cytokine.

Indeed, has been reported that sequential treatments with TGFβ blocking antibodies, 4-1BB agonist antibodies, for example, induce liver toxicity. See, e.g., Stevenson et al. Oncoimmunology. 2(8): e26218 (2013); Qi et al., Nature Communications 10: 2141 (2019); Surprisingly, treatments with a TGFBR2-Fc-4-1BBL chimeric protein blocks TGFBR2 (which inhibits the transmission of an immune inhibitory signal) and activates 4-1BB (which enhances, increases, and/or stimulates the transmission of an immune stimulatory signal). Likewise, treatments with a TGFBR2-Fc-NKG2A chimeric protein blocks TGFBR2 (which inhibits the transmission of an immune inhibitory signal) and blocks NKG2A (which also inhibits the transmission of an immune inhibitory signal). Further, in embodiments, the present chimeric proteins provide synergistic therapeutic effects (e.g., anti-tumor effects) as it allows for improved site-specific interplay of two immunotherapy agents. In embodiments, the present chimeric proteins provide synergistic therapeutic effects when compared to 4-1BB agonist antibodies and/or TGFBR2 antagonistic antibodies. In embodiments, the present chimeric proteins provide the potential for reducing off-site and/or systemic toxicity.

In embodiments, the present chimeric proteins provide reduced side-effects, e.g., GI complications, relative to current immunotherapies, e.g., antibodies directed to checkpoint molecules as described herein. Illustrative GI complications include abdominal pain, appetite loss, autoimmune effects, constipation, cramping, dehydration, diarrhea, eating problems, fatigue, flatulence, fluid in the abdomen or ascites, gastrointestinal (GI) dysbiosis, GI mucositis, inflammatory bowel disease, irritable bowel syndrome (IBS-D and IBS-C), nausea, pain, stool or urine changes, ulcerative colitis, vomiting, weight gain from retaining fluid, and/or weakness.

In embodiments, the chimeric protein is a recombinant fusion protein.

### Diseases, Methods of Treatment, and Patient Selections

In aspects, the present invention provides a method of treating a cancer, a viral infection, or an inflammatory disease, comprising administering an effective amount of a chimeric protein of any of the embodiments disclosed herein to a subject in need thereof. In aspects, the present invention provides a method of modulating a patient's immune response, comprising administering an effective amount of a chimeric protein of any of the embodiments disclosed herein to a subject in need thereof. In aspects, the present invention provides a method of treating a cancer, a viral infection, or an inflammatory disease, comprising administering an effective amount of a pharmaceutical composition of any of the embodiments disclosed herein to a subject in need thereof. In aspects, the present invention provides a method of modulating a patient's immune response, comprising administering an effective amount of a pharmaceutical composition of any of the embodiments disclosed herein to a subject in need thereof. In embodiments, the patient's T cells and or NK cells are activated. In embodiments, the patient has a tumor and one or more tumor cells are prevented from transmitting an immunosuppressive signal.

In aspects, the present invention provides a method for treating a cancer, a viral infection, or an inflammatory disease comprising administering an effective amount of a pharmaceutical composition to a subject in need thereof, the pharmaceutical composition comprising a chimeric protein comprising: (a) a first domain comprising an extracellular domain of a Type I transmembrane protein, the transmembrane protein being transforming growth factor, beta receptor II (TGFBRII), (b) a linker comprising at least one cysteine residue capable of forming a disulfide bond, and (c) a second domain comprising an extracellular domain of a Type II transmembrane protein, the transmembrane protein selected from 4-1BB Ligand (4-1BBL), CD30 Ligand (CD30L) and an NKG2 receptor.

In embodiment, the subject's T cells and/or NK cells are activated when bound by the second domain of the chimeric protein and: (a) one or more tumor cells are prevented from transmitting an immunosuppressive signal when bound by the first domain of the chimeric protein, (b) a quantifiable cytokine response in the peripheral blood of the subject is achieved, and/or (c) tumor growth is reduced in the subject in need thereof as compared to a subject treated with antibodies directed to the Type I or Type II protein, or their respective ligands or receptors. In embodiment, the method stimulates signaling of one or more of 4-1BBL, CD30L, and an NKG2 receptor and activates antigen-presenting cells. In embodiment, the method reduces the amount or activity of regulatory T cells (Tregs) as compared to untreated subjects or subjects treated with antibodies directed to the Type I or Type II protein, or their respective ligands or receptors. In embodiment, the method increases priming of effector T cells in draining lymph nodes of the subject as compared to untreated subjects or subjects treated with antibodies directed to the Type I or Type II protein, or their respective ligands or receptors. In embodiment, the method causes an overall decrease in immunosuppressive cells and a shift toward a more inflammatory tumor environment as compared to untreated subjects or subjects treated with antibodies directed to the Type I or Type II protein, or their respective ligands or receptors.

In embodiments, the present invention pertains to cancers and/or tumors; for example, the treatment or prevention of cancers and/or tumors. As described elsewhere herein, the treatment of cancer may involve in embodiments, modulating the immune system with the present chimeric proteins to favor immune stimulation over immune inhibition.

Cancers or tumors refer to an uncontrolled growth of cells and/or abnormal increased cell survival and/or inhibition of apoptosis which interferes with the normal functioning of the bodily organs and systems. Included are benign and malignant cancers, polyps, hyperplasia, as well as dormant tumors or micrometastases. Also, included are cells having abnormal proliferation that is not impeded by the immune system (e.g., virus infected cells). The cancer may be a primary cancer or a metastatic cancer. The primary cancer may be an area of cancer cells at an originating site that becomes clinically detectable, and may be a primary tumor. In contrast, the metastatic cancer may be the spread of a disease from one organ or part to another non-adjacent organ or part. The metastatic cancer may be caused by a cancer cell that acquires the ability to penetrate and infiltrate surrounding normal tissues in a local area, forming a new tumor, which may be a local metastasis. The cancer may also be caused by a cancer cell that acquires the ability to penetrate the walls of lymphatic and/or blood vessels, after which the cancer cell is able to circulate through the bloodstream (thereby being a circulating tumor cell) to other sites and tissues in the body. The cancer may be due to a process such as lymphatic or hematogeneous spread. The cancer may also be caused by a tumor cell that comes to rest at another site, re-penetrates through the vessel or walls, continues to multiply, and eventually forms another clinically detectable tumor. The cancer may be this new tumor, which may be a metastatic (or secondary) tumor.

The cancer may be caused by tumor cells that have metastasized, which may be a secondary or metastatic tumor. The cells of the tumor may be like those in the original tumor. As an example, if a breast cancer or colon cancer metastasizes to the liver, the secondary tumor, while present in the liver, is made up of abnormal breast or colon cells, not of abnormal liver cells. The tumor in the liver may thus be a metastatic breast cancer or a metastatic colon cancer, not liver cancer.

The cancer may have an origin from any tissue. The cancer may originate from melanoma, colon, breast, or prostate, and thus may be made up of cells that were originally skin, colon, breast, or prostate, respectively. The cancer may also be a hematological malignancy, which may be leukemia or lymphoma. The cancer may invade a tissue such as liver, lung, bladder, or intestinal.

Representative cancers and/or tumors of the present invention include, but are not limited to, a basal cell carcinoma, biliary tract cancer; bladder cancer; bone cancer; brain and central nervous system cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma; hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); melanoma; myeloma; neuroblastoma; oral cavity cancer (lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; lymphoma including Hodgkin's and non-Hodgkin's lymphoma, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia; chronic lymphocytic leukemia (CLL); acute lymphoblastic leukemia (ALL); Hairy cell leukemia; chronic myeloblastic leukemia; as well as other carcinomas and sarcomas; and post-transplant lymphoproliferative disorder (PTLD), as well as abnormal vascular proliferation associated with phakomatoses, edema (such as that associated with brain tumors), and Meigs' syndrome.

In embodiments, the present invention pertains to viral infections; for example, the treatment or prevention of viral infections. As described elsewhere herein, the treatment of viral infections may involve in embodiments, modulating the immune system with the present chimeric proteins to favor immune stimulation over immune inhibition.

In embodiments, the viral infection is selected from the group consisting of acute or chronic viral infections, for example, of the respiratory tract, of papilloma virus infections, of herpes simplex virus (HSV) infection, of human immunodeficiency virus (HIV) infection, and of viral infection of internal organs such as infection with hepatitis viruses. In embodiments, the viral infection is caused by a member of Flaviviridae, e.g., Yellow Fever Virus, West Nile virus, Dengue virus, Japanese Encephalitis Virus, St. Louis Encephalitis Virus, and Hepatitis C Virus; a member of Picornaviridae, e.g., poliovirus, rhinovirus, coxsackievirus; a member of Orthomyxoviridae, e.g., an influenza virus; a member of Retroviridae, e.g., a lentivirus; a member of Paramyxoviridae, e.g., respiratory syncytial virus, a human parainfluenza virus, rubulavirus (e.g., mumps virus), measles virus, and human metapneumovirus; a member of Bunyaviridae, e.g., hantavirus; or a member of Reoviridae, e.g., a rotavirus.

In embodiments, the chimeric protein is used to treat a subject that has a treatment-refractory cancer. In embodiments, the chimeric protein is used to treat a subject that is refractory to one or more immune-modulating agents. For example, in embodiments, the chimeric protein is used to treat a subject that presents no response to treatment, or even progress, after 12 weeks or so of treatment. For instance, in embodiments, the subject is refractory to a PD-1 and/or PD-L1 and/or PD-L2 agent, including, for example, nivolumab (ONO-4538/BMS-936558, MDX1106, OPDIVO, BRISTOL MYERS SQUIBB), pembrolizumab (KEYTRUDA, MERCK), pidilizumab (CT-011, CURE TECH), MK-3475 (MERCK), BMS 936559 (BRISTOL MYERS SQUIBB), I brutinib (PHARMACYCLICS/ABBVIE), atezolizumab (TECENTRIQ, GENENTECH), and/or MPDL3280A (ROCHE)-refractory patients. For instance, in embodiments, the subject is refractory to an anti-CTLA-4 agent, e.g., ipilimumab (YERVOY)-refractory patients (e.g., melanoma patients). Accordingly, in embodiments the present invention provides methods of cancer treatment that rescue patients that are non-responsive to various therapies, including monotherapy of one or more immune-modulating agents.

In embodiments, the present methods provide treatment with the chimeric protein in a patient who is refractory to an additional agent, such “additional agents” being described elsewhere herein, inclusive, without limitation, of the various chemotherapeutic agents described herein.

In embodiments, the chimeric proteins are used to treat, control or prevent one or more inflammatory diseases or conditions. Non-limiting examples of inflammatory diseases include acne vulgaris, acute inflammation, allergic rhinitis, asthma, atherosclerosis, atopic dermatitis, autoimmune disease, autoinflammatory diseases, autosomal recessive spastic ataxia, bronchiectasis, celiac disease, chronic cholecystitis, chronic inflammation, chronic prostatitis, colitis, diverticulitis, familial eosinophilia (fe), glomerulonephritis, glycerol kinase deficiency, hidradenitis suppurativa, hypersensitivities, inflammation, inflammatory bowel diseases, inflammatory pelvic disease, interstitial cystitis, laryngeal inflammatory disease, Leigh syndrome, lichen planus, mast cell activation syndrome, mastocytosis, ocular inflammatory disease, otitis, pain, pelvic inflammatory disease, reperfusion injury, respiratory disease, restenosis, rheumatic fever, rheumatoid arthritis, rhinitis, sarcoidosis, septic shock, silicosis and other pneumoconioses, transplant rejection, tuberculosis, and vasculitis.

In embodiments, the inflammatory disease is an autoimmune disease or condition, such as multiple sclerosis, diabetes mellitus, lupus, celiac disease, Crohn's disease, ulcerative colitis, Guillain-Barre syndrome, scleroderms, Goodpasture's syndrome, Wegener's granulomatosis, autoimmune epilepsy, Rasmussen's encephalitis, Primary biliary sclerosis, Sclerosing cholangitis, Autoimmune hepatitis, Addison's disease, Hashimoto's thyroiditis, Fibromyalgia, Menier's syndrome; transplantation rejection (e.g., prevention of allograft rejection) pernicious anemia, rheumatoid arthritis, systemic lupus erythematosus, dermatomyositis, Sjogren's syndrome, lupus erythematosus, multiple sclerosis, myasthenia gravis, Reiter's syndrome, Grave's disease, and other autoimmune diseases.

In aspects, the present chimeric agents are used in methods of activating an antigen presenting cell, e.g., via the extracellular domain of 4-1BBL.

In aspects, the present chimeric agents are used in methods of preventing the cellular transmission of an immunosuppressive signal via the extracellular domain of TGFBR2.

### Combination Therapies and Conjugation

In embodiments, the invention provides for chimeric proteins and methods that further comprise administering an additional agent to a subject. In embodiments, the invention pertains to co-administration and/or co-formulation. Any of the compositions described herein may be co-formulated and/or co-administered.

In embodiments, any chimeric protein described herein acts synergistically when co-administered with another agent and is administered at doses that are lower than the doses commonly employed when such agents are used as monotherapy. In embodiments, any agent referenced herein may be used in combination with any of the chimeric proteins described herein.

In embodiments, the present chimeric protein comprising the extracellular domain of TGFBR2 as described herein is co-administered with an antibody. In embodiment, the method further comprising administering to the subject an antibody that is capable of binding PD-1 or binding a PD-1 ligand. In embodiment, the chimeric protein and the antibody are administered simultaneously. In embodiment, the chimeric protein is administered after the antibody is administered. In embodiment, the chimeric protein is administered before the antibody is administered. In embodiment, the antibody is selected from the group consisting of nivolumab (ONO 4538, BMS 936558, MDX1106, OPDIVO (Bristol Myers Squibb)), pembrolizumab (KEYTRUDA/MK 3475, Merck), pidilizumab (CT 011, Cure Tech), RMP1-14, AGEN2034 (Agenus), and cemiplimab ((REGN-2810).

In embodiments, the present chimeric protein comprising the extracellular domain of TGFBR2 as described herein is co-administered with another chimeric protein. In embodiments, the present chimeric protein comprising the extracellular domain of TGFBR2 as described herein is co-administered with another chimeric protein, for example, one which modulates the adaptive immune response. In embodiments, the present chimeric protein comprising the extracellular domain of TGFBR2 as described herein is co-administered with a chimeric protein comprising one or more of 4-1BBL, CD30L, NKG2A, PD-1, GITRL, SIRPα, TIM3, TIGIT, LIGHT and VSIG8. Without wishing to be bound by theory, it is believed that a combined regimen involving the administration of the present chimeric protein which induces an innate immune response and one or more chimeric proteins which induces an adaptive immune response may provide synergistic effects (e.g., synergistic anti-tumor effects).

Any chimeric protein which induces an adaptive immune response may be utilized in the present invention. For example, the chimeric protein may be any of the chimeric proteins disclosed in U.S. 62/464,002 which induce an adaptive immune response. In such embodiments, the chimeric protein comprises a first extracellular domain of a type I transmembrane protein at or near the N-terminus and a second extracellular domain of a type II transmembrane protein at or near the C-terminus, wherein one of the first and second extracellular domains provides an immune inhibitory signal and one of the first and second extracellular domains provides an immune stimulatory signal as disclosed in U.S. 62/464,002, the entire contents of which is hereby incorporated by reference. In an exemplary embodiment, the chimeric protein which induces an adaptive immune response is a chimeric protein comprising the extracellular domain of PD-1 at the N-terminus and the extracellular domain of 4-1BBL, CD30L, NKG2A, GITRL, or 4-1BBL at the C-terminus. In an embodiment, the chimeric protein which induces an adaptive immune response is a chimeric protein comprising the extracellular domain of VSIG8 at the N-terminus and the extracellular domain of 4-1BBL, GITRL, CD30L, or NKG2A, at the C-terminus.

In embodiments, the present chimeric protein comprising the extracellular domain of TGFBR2 as described herein is administered to a patient to stimulate the innate immune response and, subsequently (e.g., 1 day later, or 2 days later, or 3 days later, or 4 days later, or 5 days later, or 6 days later, or 1 week later, or 2 weeks later, or 3 weeks later, or 4 weeks later) a chimeric protein which induce an adaptive immune response is administered.

In embodiments, inclusive of, without limitation, cancer applications, the present invention pertains to chemotherapeutic agents as additional agents. Examples of chemotherapeutic agents include, but are not limited to, alkylating agents such as thiotepa and CYTOXAN cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (e.g., bullatacin and bullatacinone); a camptothecin (including the synthetic analogue topotecan); bryostatin; cally statin; CC-1065 (including its adozelesin, carzelesin and bizelesin synthetic analogues); cryptophycins (e.g., cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN doxorubicin (including morpholino-doxorubicin, cyanomorpholino-doxorubicin, 2-pyrrolino-doxorubicin and deoxy doxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as minoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL paclitaxel (Bristol-Myers Squibb Oncology, Princeton, N.J.), ABRAXANE Cremophor-free, albumin-engineered nanoparticle formulation of paclitaxel (American Pharmaceutical Partners, Schaumberg, 111.), and TAXOTERE doxetaxel (Rhone-Poulenc Rorer, Antony, France); chloranbucil; GEMZAR gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE. vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (TYKERB); inhibitors of PKC-α, Raf, H-Ras, EGFR (e.g., erlotinib (Tarceva)) and VEGF-A that reduce cell proliferation and pharmaceutically acceptable salts, acids or derivatives of any of the above. In addition, the methods of treatment can further include the use of radiation. In addition, the methods of treatment can further include the use of photodynamic therapy.

In embodiments, inclusive of, without limitation, cancer applications, the present additional agent is one or more immune-modulating agents selected from an agent that blocks, reduces and/or inhibits PD-1 and PD-L1 or PD-L2 and/or the binding of PD-1 with PD-L1 or PD-L2 (by way of non-limiting example, one or more of nivolumab (ONO-4538/BMS-936558, MDX1106, OPDIVO, BRISTOL MYERS SQUIBB), pembrolizumab (KEYTRUDA, Merck), pidilizumab (CT-011, CURE TECH), MK-3475 (MERCK), BMS 936559 (BRISTOL MYERS SQUIBB), atezolizumab (TECENTRIQ, GENENTECH), MPDL328OA (ROCHE)), an agent that increases and/or stimulates CD137 (4-1BB) and/or the binding of CD137 (4-1BB) with one or more of 4-1BB ligand (by way of non-limiting example, urelumab (BMS-663513 and anti-4-1BB antibody), and an agent that blocks, reduces and/or inhibits the activity of CTLA-4 and/or the binding of CTLA-4 with one or more of AP2M1, CD80, CD86, SHP-2, and PPP2R5A and/or the binding of 4-1BB with 4-1BBL.

In embodiments, inclusive of, without limitation, infectious disease applications, the present invention pertains to anti-infectives as additional agents. In embodiments, the anti-infective is an anti-viral agent including, but not limited to, Abacavir, Acyclovir, Adefovir, Amprenavir, Atazanavir, Cidofovir, Darunavir, Delavirdine, Didanosine, Docosanol, Efavirenz, Elvitegravir, Emtricitabine, Enfuvirtide, Etravirine, Famciclovir, and Foscarnet. In embodiments, the anti-infective is an anti-bacterial agent including, but not limited to, cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); monobactam antibiotics (aztreonam); and carbapenem antibiotics (ertapenem, doripenem, imipenem/cilastatin, and meropenem). In embodiments, the anti-infectives include anti-malarial agents (e.g., chloroquine, quinine, mefloquine, primaquine, doxycycline, artemether/lumefantrine, atovaquone/proguanil and sulfadoxine/pyrimethamine), metronidazole, tinidazole, ivermectin, pyrantel pamoate, and albendazole.

In embodiments, inclusive, without limitation, of autoimmune applications, the additional agent is an immunosuppressive agent. In embodiments, the immunosuppressive agent is an anti-inflammatory agent such as a steroidal anti-inflammatory agent or a non-steroidal anti-inflammatory agent (NSAID). Steroids, particularly the adrenal corticosteroids and their synthetic analogues, are well known in the art. Examples of corticosteroids useful in the present invention include, without limitation, hydroxyltriamcinolone, alpha-methyl dexamethasone, beta-methyl betamethasone, beclomethasone dipropionate, betamethasone benzoate, betamethasone dipropionate, betamethasone valerate, clobetasol valerate, desonide, desoxymethasone, dexamethasone, diflorasone diacetate, diflucortolone valerate, fluadrenolone, fluclorolone acetonide, flumethasone pivalate, fluosinolone acetonide, fluocinonide, flucortine butylester, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone acetonide, medrysone, amcinafel, amcinafide, betamethasone and the balance of its esters, chloroprednisone, clocortelone, clescinolone, dichlorisone, difluprednate, flucloronide, flunisolide, fluoromethalone, fluperolone, fluprednisolone, hydrocortisone, meprednisone, paramethasone, prednisolone, prednisone, beclomethasone dipropionate. (NSAIDS) that may be used in the present invention, include but are not limited to, salicylic acid, acetyl salicylic acid, methyl salicylate, glycol salicylate, salicylmides, benzyl-2,5-diacetoxybenzoic acid, ibuprofen, fulindac, naproxen, ketoprofen, etofenamate, phenylbutazone, and indomethacin. In embodiments, the immunosupressive agent may be cytostatics such as alkylating agents, antimetabolites (e.g., azathioprine, methotrexate), cytotoxic antibiotics, antibodies (e.g., basiliximab, daclizumab, and muromonab), anti-immunophilins (e.g., cyclosporine, tacrolimus, sirolimus), inteferons, opioids, TNF binding proteins, mycophenolates, and small biological agents (e.g., fingolimod, myriocin).

In embodiments, the chimeric proteins (and/or additional agents) described herein, include derivatives that are modified, i.e., by the covalent attachment of any type of molecule to the composition such that covalent attachment does not prevent the activity of the composition. For example, but not by way of limitation, derivatives include composition that have been modified by, inter alia, glycosylation, lipidation, acetylation, pegylation, phosphorylation, amidation, derivatization by known protecting/blocking groups, proteolytic cleavage, linkage to a cellular ligand or other protein, etc. Any of numerous chemical modifications can be carried out by known techniques, including, but not limited to specific chemical cleavage, acetylation, formylation, metabolic synthesis of turicamycin, etc. Additionally, the derivative can contain one or more non-classical amino acids. In still embodiments, the chimeric proteins (and/or additional agents) described herein further comprise a cytotoxic agent, comprising, in illustrative embodiments, a toxin, a chemotherapeutic agent, a radioisotope, and an agent that causes apoptosis or cell death. Such agents may be conjugated to a composition described herein.

The chimeric proteins (and/or additional agents) described herein may thus be modified post-translationally to add effector moieties such as chemical linkers, detectable moieties such as for example fluorescent dyes, enzymes, substrates, bioluminescent materials, radioactive materials, and chemiluminescent moieties, or functional moieties such as for example streptavidin, avidin, biotin, a cytotoxin, a cytotoxic agent, and radioactive materials.

### Formulations

The chimeric proteins (and/or additional agents) described herein can possess a sufficiently basic functional group, which can react with an inorganic or organic acid, or a carboxyl group, which can react with an inorganic or organic base, to form a pharmaceutically acceptable salt. A pharmaceutically acceptable acid addition salt is formed from a pharmaceutically acceptable acid, as is well known in the art. Such salts include the pharmaceutically acceptable salts listed in, for example, Journal of Pharmaceutical Science, 66, 2-19 (1977) and The Handbook of Pharmaceutical Salts; Properties, Selection, and Use. P. H. Stahl and C. G. Wermuth (eds.), Verlag, Zurich (Switzerland) 2002, which are hereby incorporated by reference in their entirety.

In embodiments, the compositions described herein are in the form of a pharmaceutically acceptable salt.

Further, any chimeric protein (and/or additional agents) described herein can be administered to a subject as a component of a composition that comprises a pharmaceutically acceptable carrier or vehicle. Such compositions can optionally comprise a suitable amount of a pharmaceutically acceptable excipient so as to provide the form for proper administration. Pharmaceutical excipients can be liquids, such as water and oils, including those of petroleum, animal, vegetable, or synthetic origin, such as peanut oil, soybean oil, mineral oil, sesame oil and the like. The pharmaceutical excipients can be, for example, saline, gum acacia, gelatin, starch paste, talc, keratin, colloidal silica, urea and the like. In addition, auxiliary, stabilizing, thickening, lubricating, and coloring agents can be used. In one embodiment, the pharmaceutically acceptable excipients are sterile when administered to a subject. Water is a useful excipient when any agent described herein is administered intravenously. Saline solutions and aqueous dextrose and glycerol solutions can also be employed as liquid excipients, specifically for injectable solutions. Suitable pharmaceutical excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Any agent described herein, if desired, can also comprise minor amounts of wetting or emulsifying agents, or pH buffering agents.

In embodiments, the compositions described herein are suspended in a saline buffer (including, without limitation TBS, PBS, and the like).

In embodiments, the chimeric proteins may by conjugated and/or fused with another agent to extend half-life or otherwise improve pharmacodynamic and pharmacokinetic properties. In embodiments, the chimeric proteins may be fused or conjugated with one or more of PEG, XTEN (e.g., as rPEG), polysialic acid (POLYXEN), albumin (e.g., human serum albumin or HAS), elastin-like protein (ELP), PAS, HAP, GLK, CTP, transferrin, and the like. In embodiments, each of the individual chimeric proteins is fused to one or more of the agents described in BioDrugs (2015) 29:215-239, the entire contents of which are hereby incorporated by reference.

### Administration, Dosing, and Treatment Regimens

The present invention includes the described chimeric protein (and/or additional agents) in various formulations. Any chimeric protein (and/or additional agents) described herein can take the form of solutions, suspensions, emulsion, drops, tablets, pills, pellets, capsules, capsules containing liquids, powders, sustained-release formulations, suppositories, emulsions, aerosols, sprays, suspensions, or any other form suitable for use. DNA or RNA constructs encoding the protein sequences may also be used. In one embodiment, the composition is in the form of a capsule (see, e.g., U.S. Pat. No. 5,698,155). Other examples of suitable pharmaceutical excipients are described in Remington's Pharmaceutical Sciences 1447-1676 (Alfonso R. Gennaro eds., 19th ed. 1995), incorporated herein by reference.

Where necessary, the formulations comprising the chimeric protein (and/or additional agents) can also include a solubilizing agent. Also, the agents can be delivered with a suitable vehicle or delivery device as known in the art. Combination therapies outlined herein can be co-delivered in a single delivery vehicle or delivery device. Compositions for administration can optionally include a local anesthetic such as, for example, lignocaine to lessen pain at the site of the injection.

The formulations comprising the chimeric protein (and/or additional agents) of the present invention may conveniently be presented in unit dosage forms and may be prepared by any of the methods well known in the art of pharmacy. Such methods generally include the step of bringing therapeutic agents into association with a carrier, which constitutes one or more accessory ingredients. Typically, the formulations are prepared by uniformly and intimately bringing therapeutic agent into association with a liquid carrier, a finely divided solid carrier, or both, and then, if necessary, shaping the product into dosage forms of the desired formulation (e.g., wet or dry granulation, powder blends, etc., followed by tableting using conventional methods known in the art)

In one embodiment, any chimeric protein (and/or additional agents) described herein is formulated in accordance with routine procedures as a composition adapted for a mode of administration described herein.

Routes of administration include, for example: intratumoral, intradermal, intramuscular, intraperitoneal, intravenous, subcutaneous, intranasal, epidural, oral, sublingual, intranasal, intracerebral, intravaginal, transdermal, rectally, by inhalation, or topically, particularly to the ears, nose, eyes, or skin. In embodiments, the administering is effected orally or by parenteral injection. In some instances, administration results in the release of any agent described herein into the bloodstream, or alternatively, the agent is administered directly to the site of active disease.

Any chimeric protein (and/or additional agents) described herein can be administered orally. Such chimeric proteins (and/or additional agents) can also be administered by any other convenient route, for example, by intravenous infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer.

In specific embodiments, it may be desirable to administer locally to the area in need of treatment. In one embodiment, for instance in the treatment of cancer, the chimeric protein (and/or additional agents) are administered in the tumor microenvironment (e.g., cells, molecules, extracellular matrix and/or blood vessels that surround and/or feed a tumor cell, inclusive of, for example, tumor vasculature; tumor-infiltrating lymphocytes; fibroblast reticular cells; endothelial progenitor cells (EPC); cancer-associated fibroblasts; pericytes; other stromal cells; components of the extracellular matrix (ECM); dendritic cells; antigen presenting cells; T-cells; regulatory T cells; macrophages; neutrophils; and other immune cells located proximal to a tumor) or lymph node and/or targeted to the tumor microenvironment or lymph node. In embodiments, for instance in the treatment of cancer, the chimeric protein (and/or additional agents) are administered intratumorally.

In the embodiments, the present chimeric protein allows for a dual effect that provides less side effects than are seen in conventional immunotherapy (e.g., treatments with one or more of OPDIVO, KEYTRUDA, YERVOY, and TECENTRIQ). For example, the present chimeric proteins reduce or prevent commonly observed immune-related adverse events that affect various tissues and organs including the skin, the gastrointestinal tract, the kidneys, peripheral and central nervous system, liver, lymph nodes, eyes, pancreas, and the endocrine system; such as hypophysitis, colitis, hepatitis, pneumonitis, rash, and rheumatic disease.

Further, the present local administration, e.g., intratumorally, obviate adverse event seen with standard systemic administration, e.g., IV infusions, as are used with conventional immunotherapy (e.g., treatments with one or more of OPDIVO, KEYTRUDA, YERVOY, and TECENTRIQ).

Dosage forms suitable for parenteral administration (e.g., intravenous, intramuscular, intraperitoneal, subcutaneous and intra-articular injection and infusion) include, for example, solutions, suspensions, dispersions, emulsions, and the like. They may also be manufactured in the form of sterile solid compositions (e.g., lyophilized composition), which can be dissolved or suspended in sterile injectable medium immediately before use. They may contain, for example, suspending or dispersing agents known in the art.

The dosage of any chimeric protein (and/or additional agents) described herein as well as the dosing schedule can depend on various parameters, including, but not limited to, the disease being treated, the subject's general health, and the administering physician's discretion.

Any chimeric protein described herein, can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concurrently with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of an additional agent, to a subject in need thereof. In embodiments any chimeric protein and additional agent described herein are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days apart, 4 days apart, 5 days apart, 6 days apart, 1 week apart, 2 weeks apart, 3 weeks apart, or 4 weeks apart.

In embodiments, the present invention relates to the co-administration of the present chimeric protein comprising the extracellular domain of transforming growth factor beta receptor (TGFBR2) and another chimeric protein which induces an adaptive immune response. In such embodiments, the present chimeric protein may be administered before, concurrently with, or subsequent to administration of the chimeric protein which induces an adaptive immune response. For example, the chimeric proteins may be administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, 1 day apart, 2 days apart, 3 days part, 4 days apart, 5 days apart, 6 days apart, 1 week apart, 2 weeks apart, 3 weeks apart, or 4 weeks apart. In an exemplary embodiment, the present chimeric protein comprising the extracellular domain of TGFBR2 and the chimeric protein which induces an adaptive immune response are administered 1 week apart, or administered on alternate weeks (i.e., administration of the present chimeric protein comprising the extracellular domain of TGFBR2 is followed 1 week later with administration of the chimeric protein inducing an adaptive immune response and so forth).

The dosage of any chimeric protein (and/or additional agents) described herein can depend on several factors including the severity of the condition, whether the condition is to be treated or prevented, and the age, weight, and health of the subject to be treated. Additionally, pharmacogenomic (the effect of genotype on the pharmacokinetic, pharmacodynamic or efficacy profile of a therapeutic) information about a particular subject may affect dosage used. Furthermore, the exact individual dosages can be adjusted somewhat depending on a variety of factors, including the specific combination of the agents being administered, the time of administration, the route of administration, the nature of the formulation, the rate of excretion, the particular disease being treated, the severity of the disorder, and the anatomical location of the disorder. Some variations in the dosage can be expected. For administration of any chimeric protein (and/or additional agents) described herein by parenteral injection, the dosage may be about 0.1 mg to about 250 mg per day, about 1 mg to about 20 mg per day, or about 3 mg to about 5 mg per day. Generally, when orally or parenterally administered, the dosage of any agent described herein may be about 0.1 mg to about 1500 mg per day, or about 0.5 mg to about 10 mg per day, or about 0.5 mg to about 5 mg per day, or about 200 to about 1,200 mg per day (e.g., about 200 mg, about 300 mg, about 400 mg, about 500 mg, about 600 mg, about 700 mg, about 800 mg, about 900 mg, about 1,000 mg, about 1,100 mg, about 1,200 mg per day).

In embodiments, administration of the chimeric protein (and/or additional agents) described herein is by parenteral injection at a dosage of about 0.1 mg to about 1500 mg per treatment, or about 0.5 mg to about 10 mg per treatment, or about 0.5 mg to about 5 mg per treatment, or about 200 to about 1,200 mg per treatment (e.g., about 200 mg, about 300 mg, about 400 mg, about 500 mg, about 600 mg, about 700 mg, about 800 mg, about 900 mg, about 1,000 mg, about 1,100 mg, about 1,200 mg per treatment).

In embodiments, a suitable dosage of the chimeric protein (and/or additional agents) is in a range of about 0.01 mg/kg to about 100 mg/kg of body weight, or about 0.01 mg/kg to about 10 mg/kg of body weight of the subject, for example, about 0.01 mg/kg, about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, 1.9 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg body weight, inclusive of all values and ranges therebetween. In an embodiment, delivery can be in a vesicle, in particular a liposome (see Langer, 1990, Science 249:1527-1533; Treat et al., in Liposomes in therapy of Infectious Disease and Cancer, Lopez-Berestein and Fidler (eds.), Liss, New York, pp. 353-365 (1989).

Any chimeric protein (and/or additional agents) described herein can be administered by controlled-release or sustained-release means or by delivery devices that are well known to those of ordinary skill in the art. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; and 5,733,556, each of which is incorporated herein by reference in its entirety. Such dosage forms can be useful for providing controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Controlled- or sustained-release of an active ingredient can be stimulated by various conditions, including but not limited to, changes in pH, changes in temperature, stimulation by an appropriate wavelength of light, concentration or availability of enzymes, concentration or availability of water, or other physiological conditions or compounds.

In an embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105).

In an embodiment, a controlled-release system can be placed in proximity of the target area to be treated, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) may be used.

Administration of any chimeric protein (and/or additional agents) described herein can, independently, be one to four times daily or one to four times per month or one to six times per year or once every two, three, four or five years. Administration can be for the duration of one day or one month, two months, three months, six months, one year, two years, three years, and may even be for the life of the subject.

The dosage regimen utilizing any chimeric protein (and/or additional agents) described herein can be selected in accordance with a variety of factors including type, species, age, weight, sex and medical condition of the subject; the severity of the condition to be treated; the route of administration; the renal or hepatic function of the subject; the pharmacogenomic makeup of the individual; and the specific compound of the invention employed. Any chimeric protein (and/or additional agents) described herein can be administered in a single daily dose, or the total daily dosage can be administered in divided doses of two, three or four times daily. Furthermore, any chimeric protein (and/or additional agents) described herein can be administered continuously rather than intermittently throughout the dosage regimen.

### Cells and Nucleic Acids

In embodiments, the present invention provides an expression vector, comprising a nucleic acid encoding the chimeric protein described herein. In embodiments, the expression vector comprises DNA or RNA. In embodiments, the expression vector is a mammalian expression vector.

Both prokaryotic and eukaryotic vectors can be used for expression of the chimeric protein. Prokaryotic vectors include constructs based on E. coli sequences (see, e.g., Makrides, Microbiol Rev 1996, 60:512-538). Non-limiting examples of regulatory regions that can be used for expression in E. coli include lac, trp, Ipp, phoA, recA, tac, T3, T7 and λP_{L}. Non-limiting examples of prokaryotic expression vectors may include the λgt vector series such as λgt11 (Huynh et al., in “DNA Cloning Techniques, Vol. I: A Practical Approach,” 1984, (D. Glover, ed.), pp. 49-78, IRL Press, Oxford), and the pET vector series (Studier et al., Methods Enzymol 1990, 185:60-89). Prokaryotic host-vector systems cannot perform much of the post-translational processing of mammalian cells, however. Thus, eukaryotic host-vector systems may be particularly useful. A variety of regulatory regions can be used for expression of the chimeric proteins in mammalian host cells. For example, the SV40 early and late promoters, the cytomegalovirus (CMV) immediate early promoter, and the Rous sarcoma virus long terminal repeat (RSV-LTR) promoter can be used. Inducible promoters that may be useful in mammalian cells include, without limitation, promoters associated with the metallothionein II gene, mouse mammary tumor virus glucocorticoid responsive long terminal repeats (MMTV-LTR), the β-interferon gene, and the hsp70 gene (see, Williams et al., Cancer Res 1989, 49:2735-42; and Taylor et al., Mol Cell Biol 1990, 10:165-75). Heat shock promoters or stress promoters also may be advantageous for driving expression of the fusion proteins in recombinant host cells.

In embodiments, expression vectors of the invention comprise a nucleic acid encoding the chimeric proteins (and/or additional agents), or a complement thereof, operably linked to an expression control region, or complement thereof, that is functional in a mammalian cell. The expression control region is capable of driving expression of the operably linked blocking and/or stimulating agent encoding nucleic acid such that the blocking and/or stimulating agent is produced in a human cell transformed with the expression vector.

Expression control regions are regulatory polynucleotides (sometimes referred to herein as elements), such as promoters and enhancers, that influence expression of an operably linked nucleic acid. An expression control region of an expression vector of the invention is capable of expressing operably linked encoding nucleic acid in a human cell. In embodiments, the cell is a tumor cell. In an embodiment, the cell is a non-tumor cell. In embodiments, the expression control region confers regulatable expression to an operably linked nucleic acid. A signal (sometimes referred to as a stimulus) can increase or decrease expression of a nucleic acid operably linked to such an expression control region. Such expression control regions that increase expression in response to a signal are often referred to as inducible. Such expression control regions that decrease expression in response to a signal are often referred to as repressible. Typically, the amount of increase or decrease conferred by such elements is proportional to the amount of signal present; the greater the amount of signal, the greater the increase or decrease in expression.

In embodiments, the present invention contemplates the use of inducible promoters capable of effecting high level of expression transiently in response to a cue. For example, when in the proximity of a tumor cell, a cell transformed with an expression vector for the chimeric protein (and/or additional agents) comprising such an expression control sequence is induced to transiently produce a high level of the agent by exposing the transformed cell to an appropriate cue. Illustrative inducible expression control regions include those comprising an inducible promoter that is stimulated with a cue such as a small molecule chemical compound. Particular examples can be found, for example, in U.S. Pat. Nos. 5,989,910, 5,935,934, 6,015,709, and 6,004,941, each of which is incorporated herein by reference in its entirety.

Expression control regions and locus control regions include full-length promoter sequences, such as native promoter and enhancer elements, as well as subsequences or polynucleotide variants which retain all or part of full-length or non-variant function. As used herein, the term “functional” and grammatical variants thereof, when used in reference to a nucleic acid sequence, subsequence or fragment, means that the sequence has one or more functions of native nucleic acid sequence (e.g., non-variant or unmodified sequence).

As used herein, “operable linkage” refers to a physical juxtaposition of the components so described as to permit them to function in their intended manner. In the example of an expression control element in operable linkage with a nucleic acid, the relationship is such that the control element modulates expression of the nucleic acid. Typically, an expression control region that modulates transcription is juxtaposed near the 5′ end of the transcribed nucleic acid (i.e., “upstream”). Expression control regions can also be located at the 3′ end of the transcribed sequence (i.e., “downstream”) or within the transcript (e.g., in an intron). Expression control elements can be located at a distance away from the transcribed sequence (e.g., 100 to 500, 500 to 1000, 2000 to 5000, or more nucleotides from the nucleic acid). A specific example of an expression control element is a promoter, which is usually located 5′ of the transcribed sequence. Another example of an expression control element is an enhancer, which can be located 5′ or 3′ of the transcribed sequence, or within the transcribed sequence.

Expression systems functional in human cells are well known in the art, and include viral systems. Generally, a promoter functional in a human cell is any DNA sequence capable of binding mammalian RNA polymerase and initiating the downstream (3′) transcription of a coding sequence into mRNA. A promoter will have a transcription initiating region, which is usually placed proximal to the 5′ end of the coding sequence, and typically a TATA box located 25-30 base pairs upstream of the transcription initiation site. The TATA box is thought to direct RNA polymerase II to begin RNA synthesis at the correct site. A promoter will also typically contain an upstream promoter element (enhancer element), typically located within 100 to 200 base pairs upstream of the TATA box. An upstream promoter element determines the rate at which transcription is initiated and can act in either orientation. Of particular use as promoters are the promoters from mammalian viral genes, since the viral genes are often highly expressed and have a broad host range. Examples include the SV40 early promoter, mouse mammary tumor virus LTR promoter, adenovirus major late promoter, herpes simplex virus promoter, and the CMV promoter.

Typically, transcription termination and polyadenylation sequences recognized by mammalian cells are regulatory regions located 3′ to the translation stop codon and thus, together with the promoter elements, flank the coding sequence. The 3′ terminus of the mature mRNA is formed by site-specific post-translational cleavage and polyadenylation. Examples of transcription terminator and polyadenylation signals include those derived from SV40. Introns may also be included in expression constructs.

There are a variety of techniques available for introducing nucleic acids into viable cells. Techniques suitable for the transfer of nucleic acid into mammalian cells in vitro include the use of liposomes, electroporation, microinjection, cell fusion, polymer-based systems, DEAE-dextran, viral transduction, the calcium phosphate precipitation method, etc. For in vivo gene transfer, a number of techniques and reagents may also be used, including liposomes; natural polymer-based delivery vehicles, such as chitosan and gelatin; viral vectors are also suitable for in vivo transduction. In some situations, it is desirable to provide a targeting agent, such as an antibody or ligand specific for a tumor cell surface membrane protein. Where liposomes are employed, proteins which bind to a cell surface membrane protein associated with endocytosis may be used for targeting and/or to facilitate uptake, e.g., capsid proteins or fragments thereof tropic for a particular cell type, antibodies for proteins which undergo internalization in cycling, proteins that target intracellular localization and enhance intracellular half-life. The technique of receptor-mediated endocytosis is described, for example, by Wu et al., J. Biol. Chem. 262, 4429-4432 (1987); and Wagner et al., Proc. Natl. Acad. Sci. USA 87, 3410-3414 (1990).

Where appropriate, gene delivery agents such as, e.g., integration sequences can also be employed. Numerous integration sequences are known in the art (see, e.g., Nunes-Duby et al., Nucleic Acids Res. 26:391-406, 1998; Sadwoski, J. Bacteriol., 165:341-357, 1986; Bestor, Cell, 122(3):322-325, 2005; Plasterk et al., TIG 15:326-332, 1999; Kootstra et al., Ann. Rev. Pharm. Toxicol., 43:413-439, 2003). These include recombinases and transposases. Examples include Cre (Sternberg and Hamilton, J. Mol. Biol., 150:467-486, 1981), lambda (Nash, Nature, 247, 543-545, 1974), Flp (Broach, et al., Cell, 29:227-234, 1982), R (Matsuzaki, et al., J. Bacteriology, 172:610-618, 1990), cpC31 (see, e.g., Groth et al., J. Mol. Biol. 335:667-678, 2004), sleeping beauty, transposases of the mariner family (Plasterk et al., supra), and components for integrating viruses such as AAV, retroviruses, and antiviruses having components that provide for virus integration such as the LTR sequences of retroviruses or lentivirus and the ITR sequences of AAV (Kootstra et al., Ann. Rev. Pharm. Toxicol., 43:413-439, 2003). In addition, direct and targeted genetic integration strategies may be used to insert nucleic acid sequences encoding the chimeric proteins including CRISPR/CAS9, zinc finger, TALEN, and meganuclease gene-editing technologies.

In aspects, the invention provides expression vectors for the expression of the chimeric proteins (and/or additional agents) that are viral vectors. Many viral vectors useful for gene therapy are known (see, e.g., Lundstrom, Trends Biotechnol., 21: 1 17, 122, 2003. Illustrative viral vectors include those selected from Antiviruses (LV), retroviruses (RV), adenoviruses (AV), adeno-associated viruses (AAV), and a viruses, though other viral vectors may also be used. For in vivo uses, viral vectors that do not integrate into the host genome are suitable for use, such as a viruses and adenoviruses. Illustrative types of a viruses include Sindbis virus, Venezuelan equine encephalitis (VEE) virus, and Semliki Forest virus (SFV). For in vitro uses, viral vectors that integrate into the host genome are suitable, such as retroviruses, AAV, and Antiviruses. In one embodiment, the invention provides methods of transducing a human cell in vivo, comprising contacting a solid tumor in vivo with a viral vector of the invention.

In embodiments, the present invention provides a host cell, comprising the expression vector comprising the chimeric protein described herein.

Expression vectors can be introduced into host cells for producing the present chimeric proteins. Cells may be cultured in vitro or genetically engineered, for example. Useful mammalian host cells include, without limitation, cells derived from humans, monkeys, and rodents (see, for example, Kriegler in “Gene Transfer and Expression: A Laboratory Manual,” 1990, New York, Freeman & Co.). These include monkey kidney cell lines transformed by SV40 (e.g., COS-7, ATCC CRL 1651); human embryonic kidney lines (e.g., 293, 293-EBNA, or 293 cells subcloned for growth in suspension culture, Graham et al., J Gen Virol 1977, 36:59); baby hamster kidney cells (e.g., BHK, ATCC CCL 10); Chinese hamster ovary-cells-DHFR (e.g., CHO, Urlaub and Chasin, Proc Natl Acad Sci USA 1980, 77:4216); DG44 CHO cells, CHO-K1 cells, mouse sertoli cells (Mather, Biol Reprod 1980, 23:243-251); mouse fibroblast cells (e.g., NIH-3T3), monkey kidney cells (e.g., CV1 ATCC CCL 70); African green monkey kidney cells. (e.g., VERO-76, ATCC CRL-1587); human cervical carcinoma cells (e.g., HELA, ATCC CCL 2); canine kidney cells (e.g., MDCK, ATCC CCL 34); buffalo rat liver cells (e.g., BRL 3A, ATCC CRL 1442); human lung cells (e.g., W138, ATCC CCL 75); human liver cells (e.g., Hep G2, HB 8065); and mouse mammary tumor cells (e.g., MMT 060562, ATCC CCL51). Illustrative cancer cell types for expressing the fusion proteins described herein include mouse fibroblast cell line, NIH3T3, mouse Lewis lung carcinoma cell line, LLC, mouse mastocytoma cell line, P815, mouse lymphoma cell line, EL4 and its ovalbumin transfectant, E.G7, mouse melanoma cell line, B16F10, mouse fibrosarcoma cell line, MC57, and human small cell lung carcinoma cell lines, SCLC #2 and SCLC #7.

Host cells can be obtained from normal or affected subjects, including healthy humans, cancer patients, and patients with an infectious disease, private laboratory deposits, public culture collections such as the American Type Culture Collection, or from commercial suppliers.

Cells that can be used for production of the present chimeric proteins in vitro, ex vivo, and/or in vivo include, without limitation, epithelial cells, endothelial cells, keratinocytes, fibroblasts, muscle cells, hepatocytes; blood cells such as T lymphocytes, B lymphocytes, monocytes, macrophages, neutrophils, eosinophils, megakaryocytes, granulocytes; various stem or progenitor cells, in particular hematopoietic stem or progenitor cells (e.g., as obtained from bone marrow), umbilical cord blood, peripheral blood, fetal liver, etc. The choice of cell type depends on the type of tumor or infectious disease being treated or prevented, and can be determined by one of skill in the art.

Subjects and/or Animals

In embodiments, the subject and/or animal is a mammal, e.g., a human, mouse, rat, guinea pig, dog, cat, horse, cow, pig, rabbit, sheep, or non-human primate, such as a monkey, chimpanzee, or baboon. In embodiments, the subject and/or animal is a non-mammal, such, for example, a zebrafish. In embodiments, the subject and/or animal may comprise fluorescently-tagged cells (e.g., with GFP). In embodiments, the subject and/or animal is a transgenic animal comprising a fluorescent cell.

In embodiments, the subject and/or animal is a human. In embodiments, the human is a pediatric human. In embodiments, the human is an adult human. In embodiments, the human is a geriatric human. In embodiments, the human may be referred to as a patient.

In embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

In embodiments, the subject is a non-human animal, and therefore the invention pertains to veterinary use. In a specific embodiment, the non-human animal is a household pet. In another specific embodiment, the non-human animal is a livestock animal.

### Kits

The invention provides kits that can simplify the administration of any agent described herein. An illustrative kit of the invention comprises any composition described herein in unit dosage form. In one embodiment, the unit dosage form is a container, such as a pre-filled syringe, which can be sterile, containing any agent described herein and a pharmaceutically acceptable carrier, diluent, excipient, or vehicle. The kit can further comprise a label or printed instructions instructing the use of any agent described herein. The kit may also include a lid speculum, topical anesthetic, and a cleaning agent for the administration location. The kit can also further comprise one or more additional agent described herein. In one embodiment, the kit comprises a container containing an effective amount of a composition of the invention and an effective amount of another composition, such those described herein.

Any aspect or embodiment described herein can be combined with any other aspect or embodiment as disclosed herein.

The invention will be further described in the following examples, which do not limit the scope of the invention described in the claims.

### EXAMPLES

The examples herein are provided to illustrate advantages and benefits of the present technology and to further assist a person of ordinary skill in the art with preparing or using the chimeric proteins of the present technology. The examples herein are also presented in order to more fully illustrate the preferred aspects of the present technology. The examples should in no way be construed as limiting the scope of the present disclosure, as defined by the appended claims. The examples can include or incorporate any of the variations, aspects or embodiments of the present technology described above. The variations, aspects or embodiments described above may also further each include or incorporate the variations of any or all other variations, aspects or embodiments of the present technology.

### Example 1. Construction and Characterization of an Illustrative CD86- and NKG2A-Based Chimeric Protein

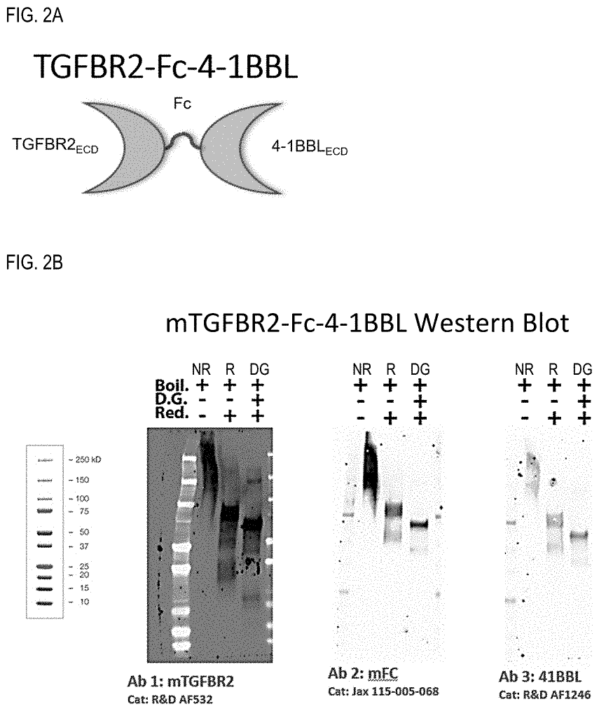

A construct encoding a murine TGFBR2- and 4-1BBL-based chimeric protein was generated. The “mTGFBR2-Fc-4-1BBL” construct included an extracellular domain (ECD) of murine TGFBR2 fused to an ECD of murine 4-1BBL via a hinge-CH2-CH3 Fc domain derived from IgG1. See, FIG. 2A.

The construct was codon optimized for expression in Chinese Hamster Ovary (CHO) cells, transfected into CHO cells and individual clones were selected for high expression. High expressing clones were then used for small-scale manufacturing in stirred bioreactors in serum-free media and the relevant chimeric fusion proteins were purified with Protein A binding resin columns.

The mTGFBR2-Fc-4-1BBL construct was transiently expressed in 293 cells and purified using protein-A affinity chromatography. To understand the native structure of the mTGFBR2-Fc-4-1BBL chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the mTGFBR2-Fc-4-1BBL chimeric protein, the gels were run in triplicates and probed using an anti-TGFBR2 antibody (FIG. 2B, left blot), an anti-Fc antibody (FIG. 2B, center blot), or an anti-4-1BBL antibody (FIG. 2B, right blot). The Western blots indicated the presence of a dominant dimer band in the non-reduced lanes (FIG. 2B, lane NR in each blot), which was reduced to a glycosylated monomeric band in the presence of the reducing agent, β-mercaptoethanol (FIG. 2B, lane R in each blot). As shown in FIG. 2B, lane DG in each blot, the chimeric protein ran as a monomer at the predicted molecular weight of about 69 kDa in the presence of both a reducing agent (β-mercaptoethanol) and a deglycosylation agent.

### Example 2: Construction and Characterization of an Illustrative TGFBR2- and NKG2A-Based Chimeric Protein

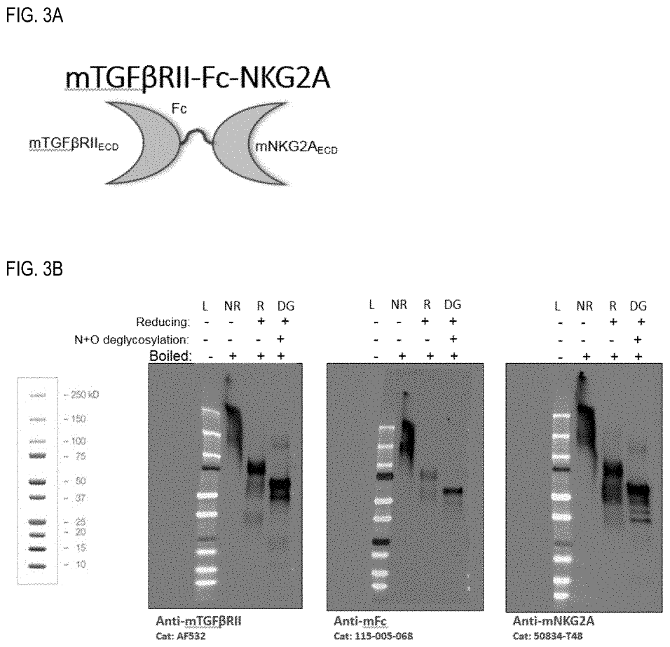

A construct encoding a mouse TGFBR2- and NKG2A-based chimeric protein was generated. The TGFBR2-Fc-NKG2A chimeric protein construct included an extracellular domain (ECD) of mouse TGFBR2 fused to an extracellular domain (ECD) of Mouse NKG2A via a hinge-CH2-CH3 Fc domain derived from IgG1. See, FIG. 3A.

The construct was expressed and purified as described in Example 1.

To understand the native structure of the mTGFBR2-Fc-NKG2A chimeric protein, untreated denatured samples (i.e., boiled in the presence of SDS, without a treatment with a reducing agent or a deglycosylation agent) were compared with (i) reduced samples, which were not deglycosylated (i.e. treated only with β-mercaptoethanol, and boiled in the presence of SDS); and (ii) reduced and deglycosylated samples (i.e. treated both with β-mercaptoethanol and a deglycosylation agent, and boiled in the presence of SDS). In addition, to confirm the presence of each domain of the TGFBR2-Fc-NKG2A chimeric protein, the gels were run in triplicates and probed using an anti-TGFBR2 antibody (FIG. 3B, left blot), an anti-Fc antibody (FIG. 3B, center blot), or an anti-NKG2A antibody (FIG. 3B, right blot). Western blot analyses were performed to confirm the presence the two ECD domains and the Fc linker of the TGFBR2-Fc-NKG2A chimeric protein with antibodies specific to the two ECD domains and the Fc linker (FIG. 3B). As shown in FIG. 3B, the Western blots indicated the presence of a dominant dimer band in the non-reduced lanes (FIG. 3B, lane NR in each blot), which was reduced to a glycosylated monomeric band in the presence of the reducing agent, β-mercaptoethanol (FIG. 3B, lane R in each blot). The chimeric protein ran as a monomer at the predicted molecular weight of about 67 kDa in the presence of both a reducing agent (β-mercaptoethanol) and a deglycosylation agent (FIG. 3B, lane DG in each blot). These results demonstrate the native state and tendency to form a dimer of the TGFBR2-Fc-NKG2A chimeric protein.

### Example 3. Detection of Fc Domains in the Illustrative TGFBR2-Based Chimeric Proteins Using ELISA

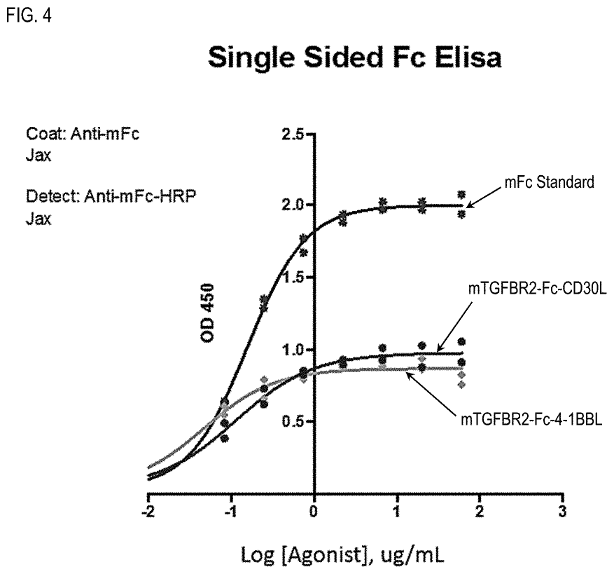

To understand whether the mouse Fc linkers from the native chimeric proteins can be accessed by anti-mouse antibodies, ELISA-based assays were performed. An anti-mFc antibody was coated on plates and increasing amounts of the mTGFBR2-Fc-CD30L and mTGFBR2-Fc-4-1BBL chimeric proteins were added to the plates for capture by the plate-bound anti-mFc antibody. Mouse Fc (mFc) IgG was used as a positive control. The binding was detected using an anti-mFc HRP. As shown in FIG. 4, dose dependent increase in signal for Fc domain of the mTGFBR2-Fc-CD30L and mTGFBR2-Fc-4-1BBL chimeric proteins suggested presence of those domains. As expected, the binding was found to be saturable. These results demonstrate that the chimeric proteins disclosed herein have an Fc domain, which can be accessed by anti-mFc antibodies.

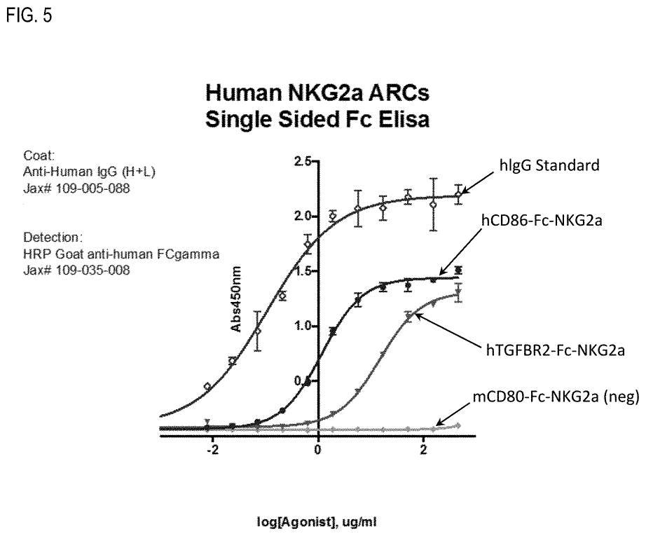

To understand whether the human Fc linkers from the native chimeric proteins can be accessed by anti-human antibodies, ELISA-based assays were performed. For these assays, an anti-human IgG antibody was coated on plates and increasing amounts of the hCD86-Fc-NKG2A, hTGFBR2-Fc-NKG2A, and mCD80-Fc-NKG2a chimeric proteins, and a human IgG antibody were added to the plates for capture by the plate-bound anti-human antibody. The human IgG antibody and the hCD86-Fc-NKG2A chimeric protein were used as a positive control. The mCD80-Fc-NKG2a chimeric protein was included as a negative control as containing a non-human Fc domain. The protein bound to the anti-human IgG antibody was detected using an anti-human Fcgamma HRP. As shown in FIG. 5, the hCD86-Fc-NKG2A and hTGFBR2-Fc-NKG2A chimeric proteins bound to the plate-bound anti-human IgG antibody in a dose-dependent manner. In contrast, the mCD80-Fc-NKG2a chimeric protein did not bind the plate-bound anti-human IgG antibody. These results demonstrate the presence of a human Fc domain and its accessibility to anti-human IgG antibodies.

These results demonstrate the presence of a Fc domains in the TGFBR2-based chimeric proteins disclosed herein, and that the Fc domains are accessible to anti-human IgG antibodies.

### Example 4. Ability of the Type II Transmembrane Protein Domain in the Illustrative TGFBR2-Based Chimeric Proteins to Bind their Natural Ligands

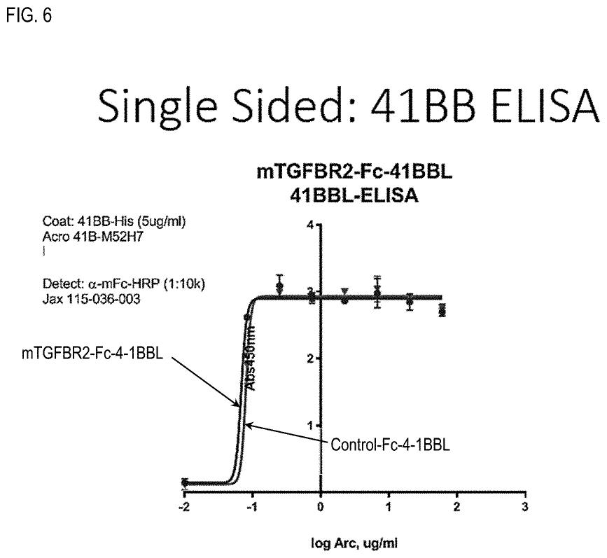

The TGFBR2-based chimeric proteins harbor type II transmembrane protein domains at or near the C-terminus. To understand whether the type II transmembrane protein domains from the native chimeric proteins can bind their natural ligands/receptors, ELISA-based assays were performed. TGFBR2-Fc-4-1BBL comprises 4-1BB ligand (4-1BBL or TNFSF9) as the type II membrane protein located at or near the C-terminus. 4-1BB (CD137, TNFRSF9) is the natural receptor/ligand of 4-1BBL. To understand whether the type II transmembrane protein domain part of the native TGFBR2-Fc-4-1BBL chimeric protein can bind 4-1BB, ELISA-based assays were performed. Recombinant 4-1BB-His protein was coated on plates. Increasing amounts of the TGFBR2-Fc-4-1BBL chimeric protein, or a positive control protein comprising Fc-4-1BBL (Control-Fc-4-1BBL) were added for capture by the plate-bound 4-1BB-His protein. Binding was detected using an anti mFc-HRP antibody. As shown in FIG. 6, the TGFBR2-Fc-4-1BBL and Control-Fc-4-1BBL chimeric proteins bound to the plate-bound 4-1BBL protein. Therefore, the type II transmembrane protein domain of the TGFBR2-Fc-4-1BBL and Control-Fc-4-1BBL chimeric proteins bind to the natural ligand/receptor of the 4-1BBL.

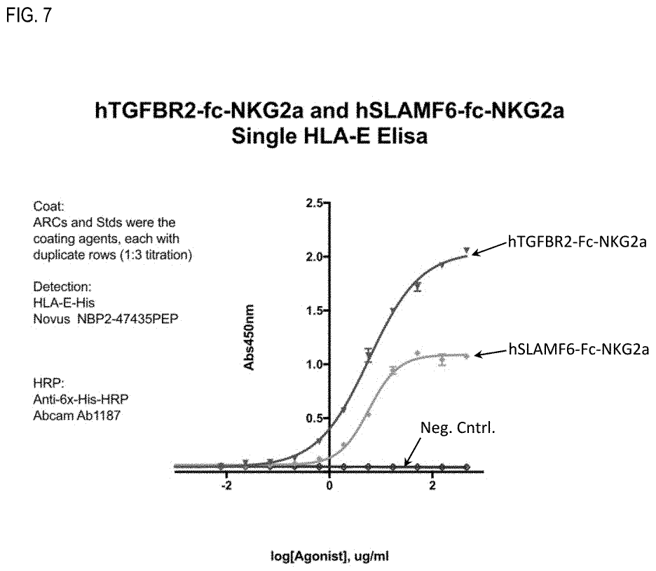

TGFBR2-Fc-NKG2A comprises NK group 2 member A (NKG2A) as the type II membrane protein located at or near the C-terminus. NKG2A binds to a nonclassical minimally polymorphic HLA class I molecule (HLA-E), which presents peptides derived from other HLA class I molecules. To understand whether the NKG2A part of the chimeric proteins disclosed herein can bind HLA-E, ELISA-based assays were performed. Briefly, increasing amounts of the human TGFBR2-Fc-C and hSLAMF6-Fc-NKG2A chimeric proteins or a negative control protein lacking CD94 were coated on plates. The hSLAMF6-Fc-NKG2A chimeric protein was used a positive control. Recombinant HLA-E-His protein was added to the plates for capture by the plate-bound chimeric proteins. The captured HLA-E-His protein was detected using an anti-6X-His-HRP antibody. As shown in FIG. 7, a dose-dependent binding of the hTGFBR2-Fc-NKG2A and hSLAMF6-Fc-NKG2A chimeric proteins to HLA-E was observed. Therefore, the NKG2A part of the chimeric proteins disclosed herein specifically bind their natural ligand.

These results demonstrate that the TGFBR2-based chimeric proteins disclosed herein bind to the natural ligand/receptors of the type II transmembrane protein domain located at their C-terminus.

### Example 5: The Ability of the TGFBR2-Based Chimeric Proteins to Bind Transforming Growth Factor (TGFβ1)

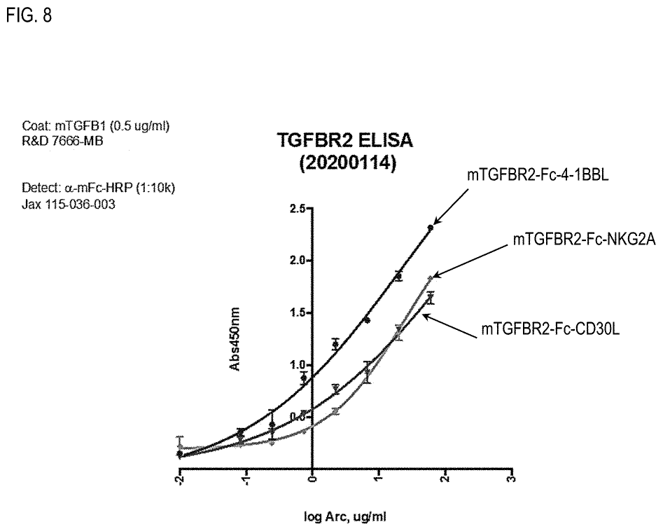

TGFβ1 is a ligand of TGFBR2. To understand whether the TGFBR2 part of the TGFBR2-based chimeric proteins disclosed herein can bind TGFβ1, ELISA-based assays were performed. Briefly, recombinant TGFβ1 protein was coated on plates and increasing amounts of the TGFBR2-Fc-4-1BBL, TGFBR2-Fc-CD30L, and TGFBR2-Fc-NKG2A chimeric proteins were added to the plates for capture by the plate-bound recombinant TGFβ1 protein. The captured TGFBR2-based chimeric proteins were detected using an anti mFc-HRP antibody. As shown in FIG. 8, the TGFβ1-bound TGFBR2-Fc-4-1BBL, TGFBR2-Fc-CD30L, and TGFBR2-Fc-NKG2A chimeric proteins increased in a dose-dependent manner. These results demonstrate that TGFBR2 part of the TGFBR2-based chimeric proteins disclosed herein bind to the natural ligand TGFβ1.

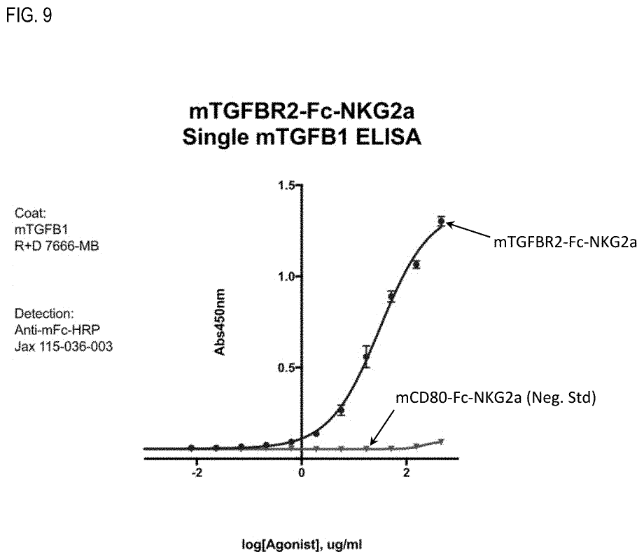

In another such experiment, recombinant TGFβ1 protein was coated on plates and increasing amounts of the TGFBR2-Fc-NKG2A and CD80-Fc-NKG2A chimeric proteins were added to the plates for capture by the plate-bound recombinant TGFβ1 protein. The CD80-Fc-NKG2A chimeric protein was included as a negative control. The captured chimeric proteins were detected using an anti mFc-HRP antibody. As shown in FIG. 9, each of the TGFBR2-Fc-NKG2A chimeric protein bound to the plate-bound recombinant TGFβ1 protein in a dose-dependent manner. These results demonstrate that the TGFBR2 part of the TGFBR2-based chimeric proteins disclosed herein specifically binds to TGFβ1 protein.

These results demonstrate that the TGFBR2 part of the TGFBR2-based chimeric proteins disclosed herein bind to the natural ligand TGFβ1 in a dose-dependent manner.

### Example 6. Kinetics of Binding to Natural Ligands by the Type II Transmembrane Protein Domain and TGFBR2 Parts of the Illustrative TGFBR2-Based Chimeric Proteins

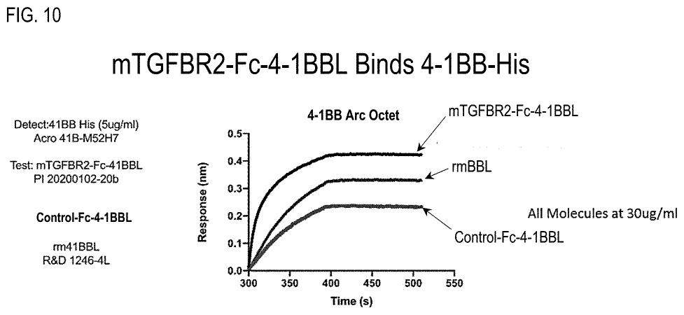

To study the binding kinetics of type II transmembrane protein domain and TGFBR2 parts of the illustrative TGFBR2-based chimeric proteins, binding assays were performed using the Octet system (ForteBio). Binding of the TGFBR2-Fc-4-1BBL chimeric protein to recombinant 4-1BB was analyzed. Briefly, recombinant 4-1BB-His protein was immobilized and detected using the mTGFBR2-Fc-4-1BBL chimeric protein, a positive control protein comprising Fc-4-1BBL (Control-Fc-4-1BBL) or commercially available recombinant mouse 4-1 BB Ligand protein (R&D Systems, Cat. No. 1246-4L). The binding response of the mTGFBR2-Fc-4-1BBL chimeric protein to 4-1BB-His protein was measured and plotted in real time on a sensorgram trace. As shown in FIG. 10, the mTGFBR2-Fc-4-1BBL chimeric protein bound to m4-1BB-His with a higher K_{D} than the commercially available recombinant mouse 4-1 BB Ligand protein.

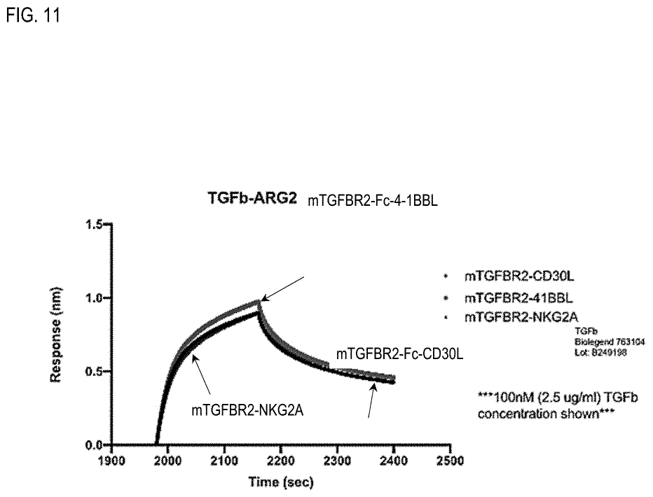

In another experiment, binding of the TGFBR2-based chimeric proteins to recombinant TGFβ1 was analyzed. Varying amounts of the recombinant Mouse TGFβ1 protein (BioLegend 763104) were covalently linked to amine-reactive (ARG2, ForteBio) OCTET tips and detected using 5 μg/ml each of the TGFBR2-Fc-4-1BBL, TGFBR2-Fc-CD30L, and TGFBR2-Fc-NKG2A chimeric proteins. FIG. 11 shows the data obtained with 100 nM recombinant Mouse TGFβ1 protein are shown. As shown in FIG. 11, the TGFBR2-Fc-4-1BBL, TGFBR2-Fc-CD30L, and TGFBR2-Fc-NKG2A chimeric proteins bound to TGFβ1 protein with similar kinetics.

These results demonstrate that both the TGFBR2 part, located at or near N-terminus, and the type II transmembrane protein domain, located at or near C-terminus, of the TGFBR2-based chimeric proteins disclosed herein bind to the natural ligand with expected kinetic parameters.

### Example 7. Sequestration of TGFβ1 by the TGFBR2-Based Chimeric Proteins

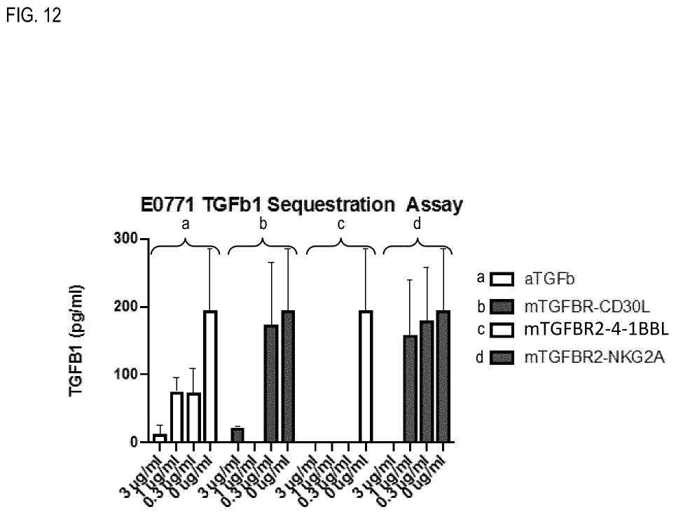

To determine the ability of the TGFBR2-based chimeric proteins to bind to and sequester TGFβ1 in a cell supernatant, TGFβ1 expressing tumor cell line EO771 was used. Briefly, EO771 cells were plated at 200,000 cfu/well in 500 μl and incubated overnight. The EO771 cells were treated with increasing amounts (0.3 μg/ml, 1 μg/ml and 3 μg/ml) of an anti-TGFB1 antibody, or the mTGFBR2-fc-4-1BBL, mTGFBR2-fc-CD30L, and mTGFBR2-fc-NKG2a chimeric proteins. Incubation was continued for another 8 hours. Cell culture supernatents were collected, frozen and stored at −20° C. TGFβ1 in the samples was assayed in duplicates using the DuoSet ELISA kit (R&D Cat. No. DY1679-05) to determine the concentration of free TGFβ1 in the cell culture. As shown in FIG. 12, each of the TGFBR2-based chimeric protein was able to sequester the secreted TGFβ1 protein. These results demonstrate that the TGFBR2-based chimeric proteins disclosed herein sequester TGFβ1.

### Example 8. Inhibition of TGFβ Signalling by the TGFBR2-Based Chimeric Proteins

To assay the effect of the TGFBR2-based chimeric proteins, the HEK BLUE TGF-β cells (Invivogen) were used. These cells release a quantifiable molecule into the supernatant when TGFβ signaling is activated. The HEK BLUE TGF-β cells were exposed to an anti-TGFB1 antibody or the mTGFBR2-Fc-4-1BBL chimeric protein for 24 hours. At the same time, 100 ng/ml TGFβ1 was added to the cells to initiate TGFβ signaling.

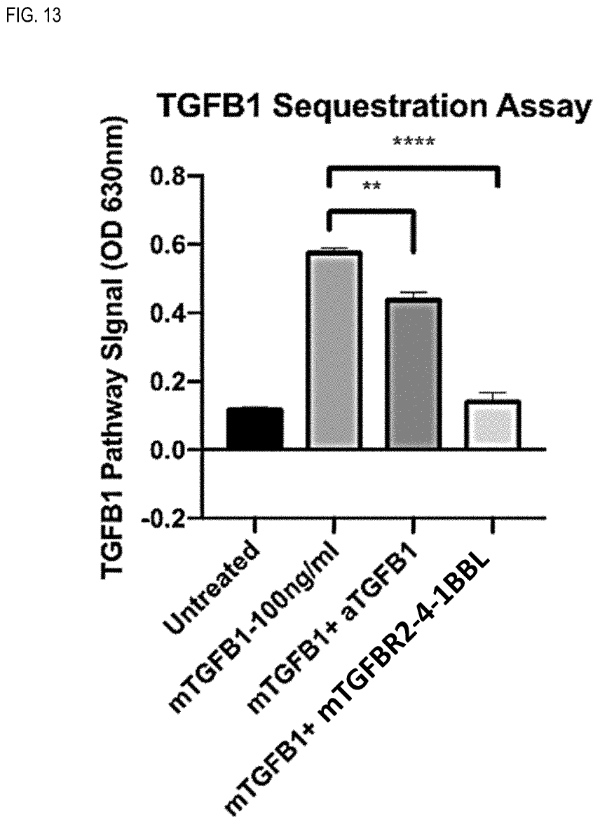

Aliquots of culture supernatant were removed and the signal for the quantifiable molecule was measured on a plate reader. In this assay, the amount of positive signal is proportional to the extent of signaling downstream to TGFβ. Compared to untreated cells, the cells treated with 100 ng/ml TGFβ1 alone showed increased TGFβ signaling (FIG. 13). As shown in FIG. 13, the anti-TGFB1 antibody and the mTGFBR2-Fc-4-1BBL chimeric protein significantly inhibited the TGFβ signaling compared to the TGFβ1 treated cells. The mTGFBR2-Fc-4-1BBL chimeric protein inhibited TGFβ signaling more than the anti-TGFB1 antibody (FIG. 13). These results demonstrate that the TGFBR2-based chimeric proteins disclosed herein inhibit the TGFβ1 signaling significantly more potently than that by the anti-TGFB1 antibody.

### Example 9: Specific Binding of the TGFBR2-Based Chimeric Proteins to Cells Expressing the Natural Ligands by the Type II Transmembrane Protein Domain

To understand whether the 4-1BBL part of the mTGFBR2-Fc-4-1BBL chimeric protein can specifically bind cells expressing 4-1BB, cells expressing 4-1 BB were generated. Specifically, the CHO-K1 cells expressing 4-1 BB were generated using standard techniques. Clone 20, a positive clone, called CHO-K1/m4-1BB+ (C20), was used for further studies. To determine whether the mTGFBR2-Fc-4-1BBL chimeric protein can specifically bind the CHO-K1/4-1BB cells, a flow-cytometry-based assay was carried out. CHO-K1/m4-1BB+(C20) and WT CHO-K1 cells were stained with increasing concentrations of the TGFBR2-Fc-4-1BBL chimeric protein, or positive control protein comprising Fc-4-1BBL (Control-Fc-4-1BBL) and analyzed by flow cytometry. Unstained CHO-K1/m4-1BB+ (C20) and WT CHO-K1 cells or CHO-K1/m4-1BB+ (C20) and WT CHO-K1 cells stained without the TGFBR2-Fc-4-1BBL chimeric protein, or positive control protein comprising Fc-4-1BBL (Control-Fc-4-1BBL) were used as control.

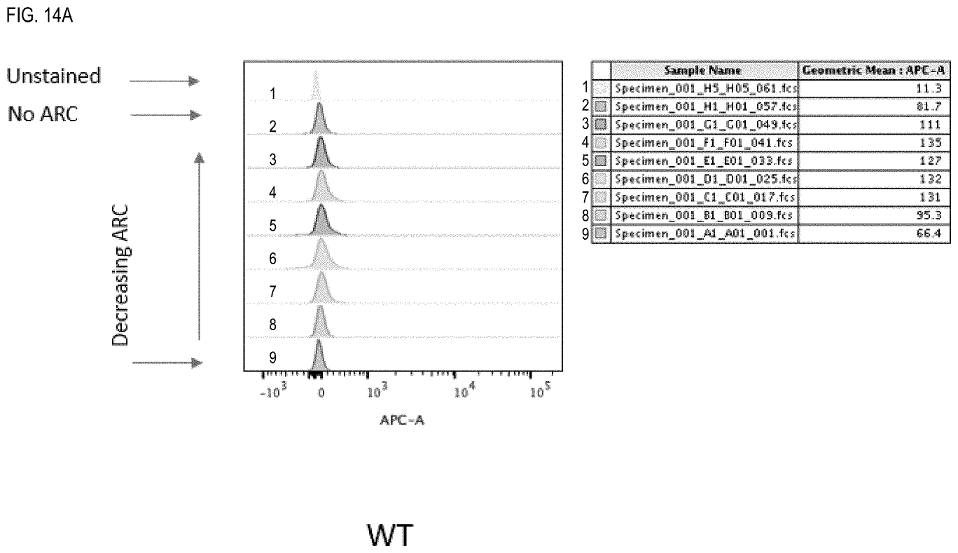

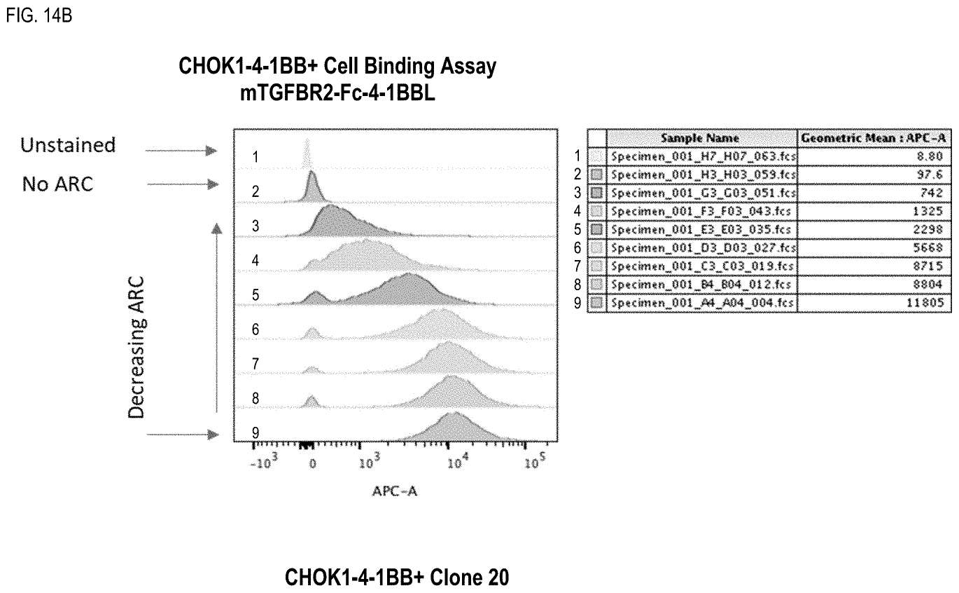

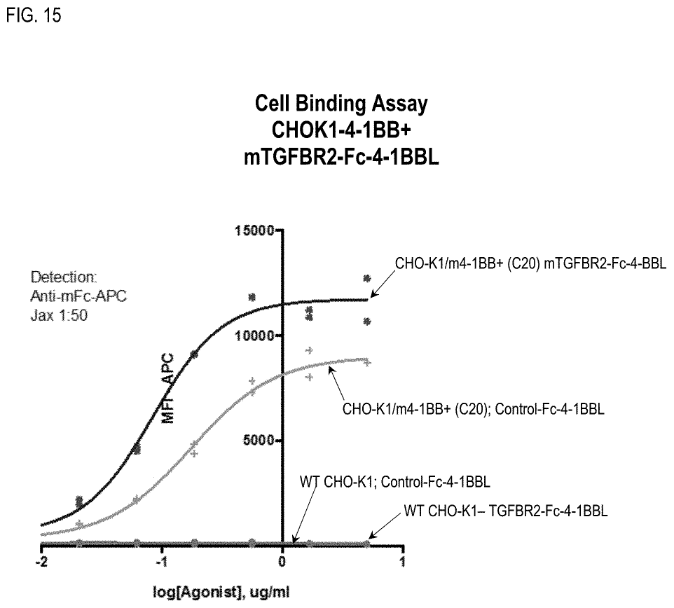

As shown in FIG. 14A and FIG. 14B, the mTGFBR2-Fc-4-1BBL chimeric protein displayed more binding to CHO-K1/4-1BB cells (FIG. 14B) compared to the WT CHO-K1 cells (FIG. 14A). The dose dependent shifts in the flow cytometry profiles illustrate a dose-dependent binding of the mTGFBR2-Fc-4-1BBL chimeric protein to 4-1BB expressed by the CHO-K1/4-1BB cells. A quantitation of binding confirmed a dose-dependent and saturable binding by both mTGFBR2-Fc-4-1BBL chimeric protein and Control-Fc-4-1BBL protein (FIG. 15). These results demonstrate that the TGFBR2-based chimeric proteins disclosed herein inhibit binds cells expressing 4-1BB in a dose-dependent and saturable manner.

### Example 10: The Ability of the Type II Transmembrane Protein Part of the TGFBR2-Based Chimeric Proteins to Initiate Signal Transduction Downstream to their Ligands

To understand whether the 4-1BBL part of the TGFBR2-based chimeric proteins can initiate signaling downstream to the ligand/receptor of the type II transmembrane protein part, clones of CHO-K1 cells expressing mQa1 (the binding partner of mouse NKG2A) were generated. The CHO-K1 cell clones used in these assays harbor an NFκB-luciferase reporter that is sensitive to the binding of a ligand to the ligands that the CHO-K1 cell clones express.

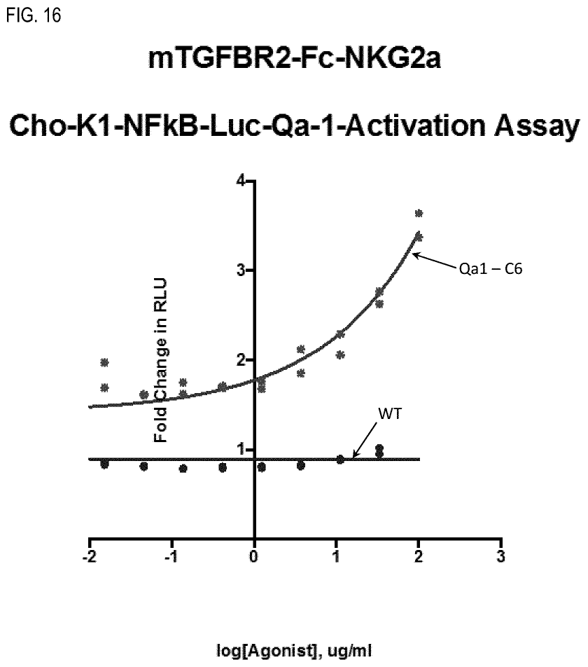

To understand whether the TGFBR2-Fc-NKG2A chimeric protein can activate Qa1 signaling, a luciferase assay was performed. Briefly, increasing amounts of the TGFBR2-Fc-NKG2A chimeric protein was incubated with the CHO-K1/mQa1 cells, or WT CHO-K1 cells, and the activation of the mQa1 was measured by a luciferase assay. As shown in FIG. 16, the TGFBR2-Fc-NKG2A chimeric protein induced luciferase activity in CHO-K1/mQa1 cells, but not WT CHO-K1 cells, in a dose dependent manner. Therefore, the TGFBR2-Fc-NKG2A chimeric protein activates mQa1 signaling.

These results demonstrate that the type II transmembrane protein part, which is located at or near the C-terminus of TGFBR2-based chimeric proteins disclosed herein activate their natural ligands in a dose-dependent manner.

### Example 11. Induction of the Activated T Cell-Mediated Killing of Antigen Positive Cells by the TGFBR2-Based Chimeric Proteins

The objective of this experiment was to understand whether the human chimeric proteins disclosed herein can induce killing of antigen positive cells mediated by the antigen activated T cells. An assay to determine if the TGFBR2-based chimeric proteins can enhance antigen specific anti-tumor response when exposed to antigen activated T cells (OT-1 naïve T cells) was developed. Target cells were antigen positive (OVA+) EO771 cells.

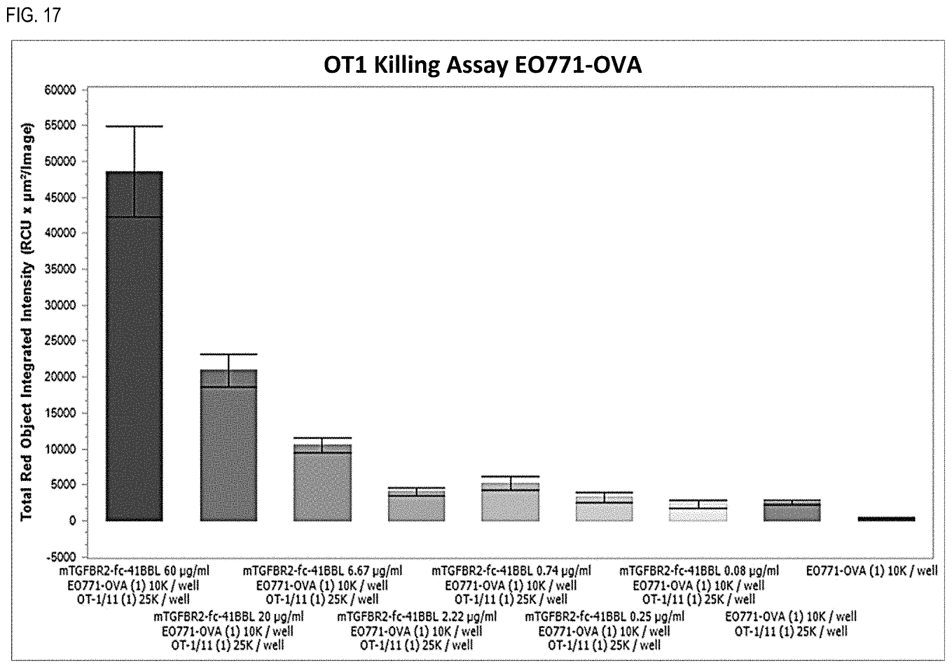

Increasing amounts of the TGFBR2-Fc-4-1BBL chimeric protein was incubated with 25,000 cells OT-1 naïve T cells per well (effector cells) and 10,000 cells EO771 OVA+ cells per well (target cells). EO771 OVA+ cells alone and EO771 OVA+ cells+OT-1 naïve T cells (without the TGFBR2-Fc-4-1BBL chimeric protein) were included as negative controls. Apoptosis was assessed by measuring caspase 3/7 activity using the INCUCYTE system, which allows live-cell imaging and fluorescent signaling of caspase 3/7 (an apoptosis marker). As shown in FIG. 17, EO771 OVA+ cells alone showed detectable levels of apoptosis. OT-1 naïve T cells modestly increased the apoptosis of EO771 OVA+ cells apoptosis in absence of the TGFBR2-Fc-4-1BBL chimeric protein (FIG. 17). The TGFBR2-Fc-4-1BBL chimeric protein induced a dose-dependent increase in apoptosis. These data demonstrate that the TGFBR2-Fc-4-1BBL chimeric protein enhanced the apoptosis of target cells when exposed to effector cells in a dose dependent manner.

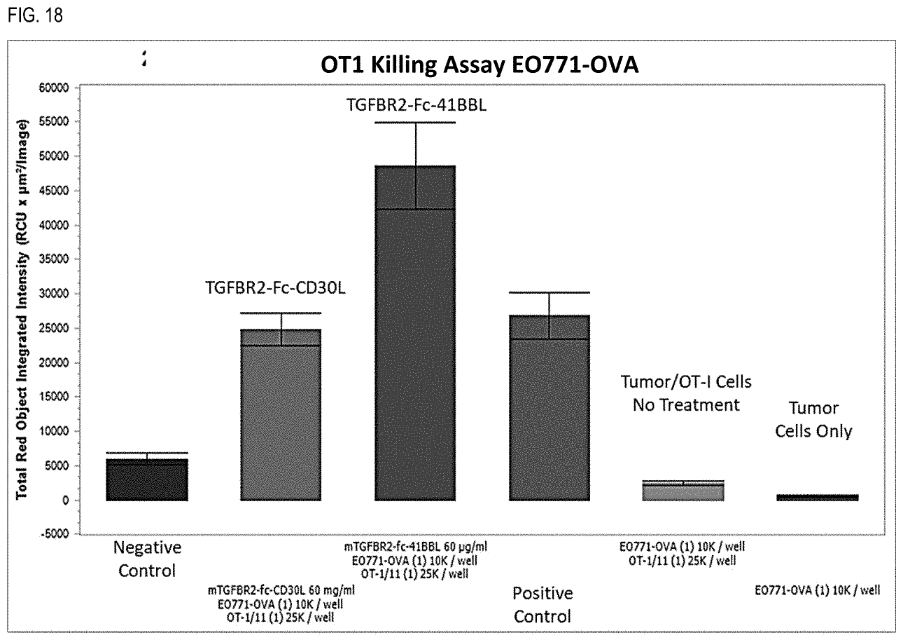

To investigate whether apoptosis induced by other TGFBR2-based chimeric protein, the TGFBR2-Fc-4-1BBL and TGFBR2-Fc-CD30L chimeric proteins, and a positive control, which induces apoptosis in this system, were incubated with 25,000 cells OT-1 naïve T cells per well (effector cells) and 10,000 cells EO771 OVA+ cells per well (target cells). 0771 OVA+ cells alone and EO771 OVA+ cells+OT-1 naïve T cells (without the TGFBR2-Fc-4-1BBL chimeric protein) were included as negative controls. As shown in FIG. 18, EO771 OVA+ cells alone showed detectable levels of apoptosis. OT-1 naïve T cells modestly increased the apoptosis of EO771 OVA+ cells apoptosis in absence of the TGFBR2-Fc-4-1BBL chimeric protein (FIG. 18). The TGFBR2-Fc-4-1BBL and TGFBR2-Fc-CD30L chimeric proteins induced apoptosis which is equivalent to more than the positive control.

These data demonstrate that the TGFBR2-based chimeric protein disclosed herein enhance activated t cell-mediated killing of antigen positive cells.

### Example 12: In Vivo Efficacy of the Chimeric Proteins of the Present Disclosure Against Triple Negative Breast Cancer Allografts

The objective of this experiment was to study the efficacy of the TGFBR2-based chimeric proteins disclosed herein against cancer. Efficacy of several chimeric proteins was studied in comparison with an anti-PD-1 antibody and anti-NKG2a antibody in murine triple negative breast cancer cell line EO771 allografts.

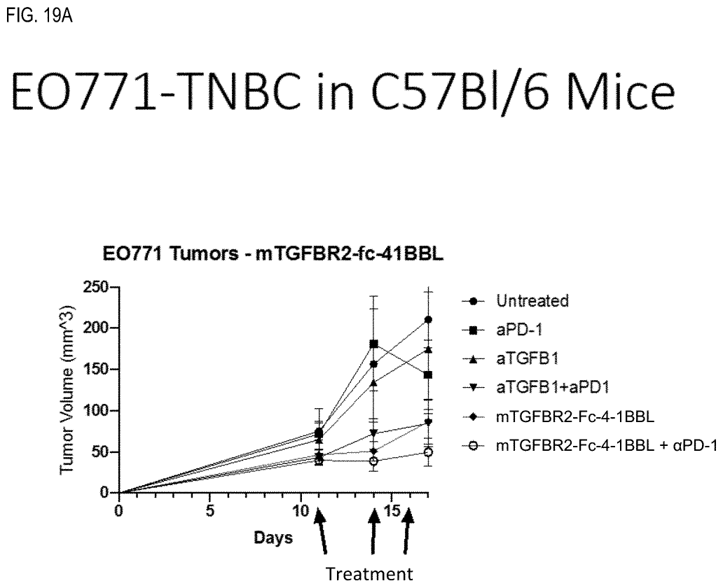

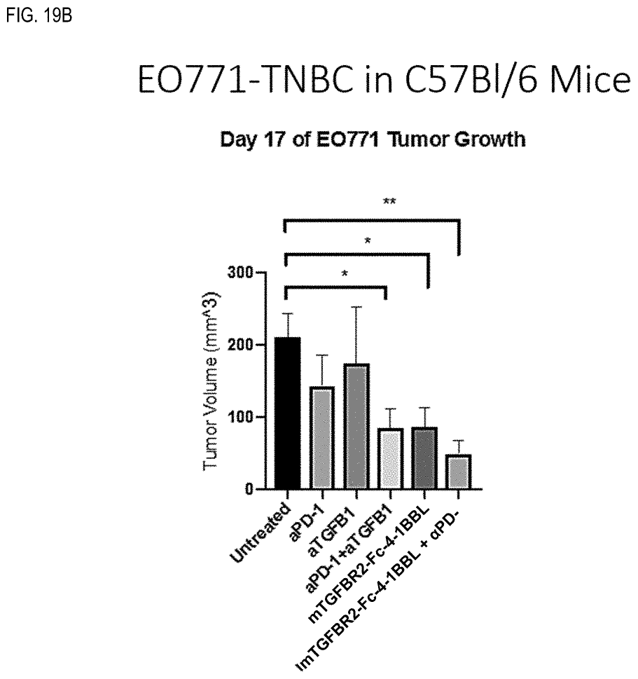

The effect of the TGFBR2-Fc-4-1BBL chimeric protein on EO771 allografts was studied in comparison with an anti-PD-1 antibody and anti-TGFB1 antibody. Briefly, Balb/c mice were inoculated with EO771 cells. After the tumors were established, the mice were randomly assigned to the following treatment groups: (1) untreated, (2) 100 μg/mouse of an anti-PD1 antibody (Bioxcell clone RMP1-14), (3) 100 μg/mouse of an anti-TGFB1 antibody (clone 1D11.16.8) and (4) 300 μg/mouse of the TGFBR2-Fc-4-1BBL chimeric protein. The mice were treated three additional times. The treatment was administered on on days 11, 14, and 16 post-inoculation. Tumor volumes were measured. As shown in FIG. 19A, the treatment with the TGFBR2-Fc-4-1BBL chimeric protein retarded the tumor growth compared to the untreated mice. The treatment with anti-PD1 and anti-TGFB1 antibodies alone did not have a significant effect but their combination a significant effect. Significantly, the combination of TGFBR2-Fc-4-1BBL chimeric protein and anti-TGFB1 antibody caused highest tumor size reduction Tumor volumes on day 18 are plotted in FIG. 19B. The treatment with the TGFBR2-Fc-4-1BBL chimeric protein significant reduction in tumor volume compared to the tumors in both untreated and anti-PD-1 antibody-treated mice. These results demonstrate that the mTGFBR2-Fc-4-1BBL chimeric protein significantly reduced tumor growth and outperformed the anti-PD-1 or anti-TGFB1 antibodies. Additionally, the combination of TGFBR2-Fc-4-1BBL chimeric protein and anti-TGFB1 antibody further improved the efficacy.

These results demonstrate that the TGFBR2-based chimeric proteins disclosed herein are effective in significantly slowing down tumor growth. Moreover, the combination of the TGFBR2-based chimeric proteins disclosed herein with an anti-PD1 antibody enhance the antitumor activity of the TGFBR2-based chimeric proteins disclosed herein.

### Example 13: In Vivo Efficacy of the Chimeric Proteins of the Present Disclosure Against Myelomonocytic Leukemia Allografts

The objective of this experiment was to further study the efficacy of the chimeric proteins disclosed herein against cancer. Efficacy of several chimeric proteins was studied in comparison with an anti-PD-1 antibody and anti-NKG2a antibody in murine myelomonocytic leukemia cell line WEHI-3 allografts.

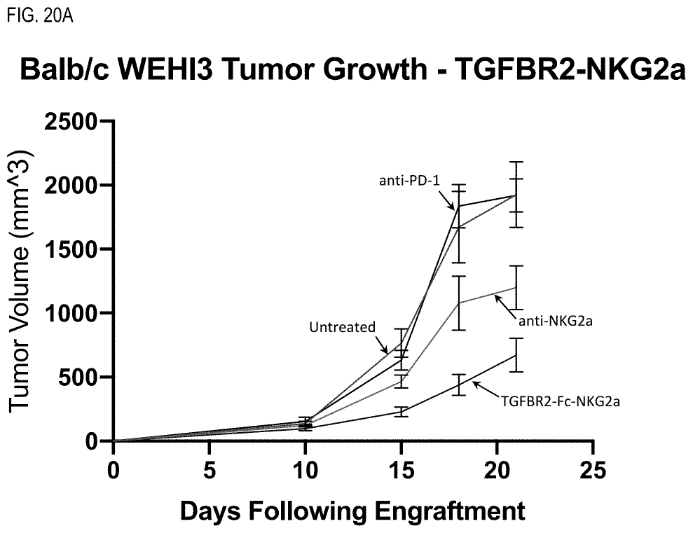

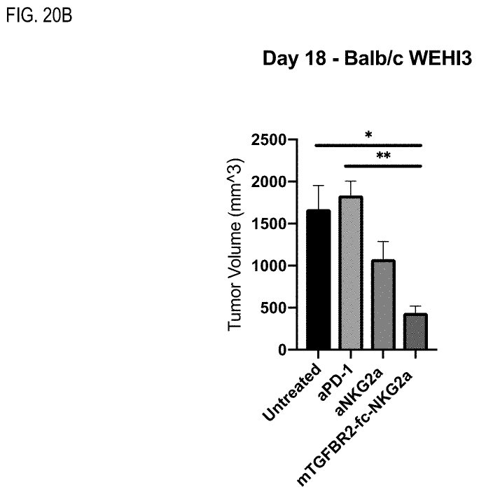

The effect of the TGFBR2-Fc-NKG2A chimeric protein on WEHI-3 allografts was studied in comparison with an anti-PD-1 antibody and anti-NKG2a antibody. Briefly, Balb/c mice were inoculated with WEHI-3 cells. After the tumors were established, the mice were randomly assigned to the following treatment groups: (1) untreated, (2) 100 μg/mouse of an anti-PD1 antibody (Bioxcell clone RMP1-14), (3) 100 μg/mouse of an anti-NKG2A antibody (BioXcell clone 20D5) and (4) 300 μg/mouse of the TGFBR2-Fc-NKG2A chimeric protein. The mice were treated on days 0, 2, 4, 6, 8 and 10 post-inoculation. The mice were treated six times, two days apart. Tumor volumes were measured. As shown in FIG. 20A, each of the treatments with anti-NKG2A antibody or the TGFBR2-Fc-NKG2A chimeric protein retarded the tumor growth compared to the untreated mice. The treatment with anti-PD1 antibody did not have a significant effect. The TGFBR2-Fc-NKG2A chimeric protein caused higher tumor size reduction compared to anti-PD1 antibody or anti-NKG2A antibody. Tumor volumes on day 18 are plotted in FIG. 20B. The treatment with the TGFBR2-Fc-NKG2A chimeric protein significant reduction in tumor volume compared to the tumors in both untreated and anti-PD-1 antibody-treated mice. These results demonstrate that the mTGFBR2-Fc-NKG2A chimeric protein significantly reduced tumor growth and outperformed the anti-PD-1 antibody. These results demonstrate that the TGFBR2-Fc-NKG2A chimeric protein outperformed the anti-PD-1 and anti-NKG2A antibodies in reducing the tumor volume of WEHI3 tumors.

### INCORPORATION BY REFERENCE

All patents and publications referenced herein are hereby incorporated by reference in their entireties.

The publications discussed herein are provided solely for their disclosure prior to the filing date of the present application. Nothing herein is to be construed as an admission that the present technology is not entitled to antedate such publication by virtue of prior invention.

As used herein, all headings are simply for organization and are not intended to limit the disclosure in any manner. The content of any individual section may be equally applicable to all sections.

### EQUIVALENTS

While the invention has been disclosed in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

Those skilled in the art will recognize, or be able to ascertain, using no more than routine experimentation, numerous equivalents to the specific embodiments disclosed specifically herein. Such equivalents are intended to be encompassed in the scope of the following claims.

1. A chimeric protein of a general structure of:
N terminus-(a)-(b)-(c)-C terminus,
wherein:
(a) is a first domain comprising an extracellular domain of a Type I transmembrane protein, the transmembrane protein being transforming growth factor, beta receptor II (TGFBRII),
(b) is a linker comprising at least one cysteine residue capable of forming a disulfide bond, and
(c) is a second domain comprising an extracellular domain of Type II transmembrane protein, the transmembrane protein being selected from 4-1BB Ligand (4-1BBL), CD30 Ligand (CD30L) and an NKG2 receptor, wherein:
the linker connects the first domain and the second domain and optionally comprises one or more joining linkers.

2. The chimeric protein of claim 1, wherein the second domain comprises an extracellular domain of 4-1BBL or CD30L.

3. (canceled)

4. The chimeric protein of claim 1, wherein the second domain is capable of binding 4-1BB or CD30.

5.-8. (canceled)

9. The chimeric protein of claim 4, wherein the second domain is capable of:
costimulating CD4 and/or CD8 T-cells, and/or
enhancing T-cell activation, proliferation and cytokine production.

10. (canceled)

11. The chimeric protein of claim 1, wherein the second domain comprises an extracellular domain of NKG2A.

12. (canceled)

13. The chimeric protein of claim 11, wherein the second domain is capable of binding HLA-E.

14. (canceled)

15. The chimeric protein of claim 13, wherein binding to the NKG2A ligand blocks transmission of an immune inhibitory signal to an NK cell.

16. (canceled)

17. The chimeric protein of claim 1, wherein the TGFBRII binds to a transforming growth factor (TGFβ).

18. The chimeric protein of claim 17, wherein the TGFβ is TGFβ3 and/or TGFβ1.

19. The chimeric protein of claim 1, wherein the binding to the TGFβ inhibits signaling by TGFβ3 and/or TGFβ1.

20. (canceled)

21. The chimeric protein of claim 1, wherein the linker comprises hinge-CH2-CH3 Fc domain derived from IgG4.

22. The chimeric protein of claim 1, wherein the linker comprises hinge-CH2-CH3 Fc domain derived from IgG1.

23. The chimeric protein of claim 21, wherein the linker comprises an amino acid sequence that is at least 95% identical to the amino acid sequence of SEQ ID NO: 25, SEQ ID NO: 26, or SEQ ID NO: 27.

24. The chimeric protein of claim 23, wherein the linker comprises one or more joining linkers independently selected from SEQ ID NOs: 28-74.

25. The chimeric protein of claim 24, wherein the linker comprises two or more joining linkers, each joining linker independently selected from SEQ ID NOs: 28-74; wherein one joining linker is N terminal to the hinge-CH2-CH3 Fc domain and another joining linker is C terminal to the hinge-CH2-CH3 Fc domain.

26. The chimeric protein of claim 1, wherein the chimeric protein is a recombinant fusion protein.

27.-38. (canceled)

39. The chimeric protein of claim 1, wherein:
the extracellular domain of TGFBRII comprises protein has an amino acid sequence that is at least 95% identical to the amino acid sequence of one of SEQ ID NO: 2;
the extracellular domain of 4-1BB ligand (4-1BBL) comprises protein has an amino acid sequence that is at least 95% identical to the amino acid sequence of one of SEQ ID NO: 4;
the extracellular domain of CD30 ligand (CD30L) comprises protein has an amino acid sequence that is at least 95% identical to the amino acid sequence of one of SEQ ID NO: 6; and/or
the extracellular domain of NKG2A comprises protein has an amino acid sequence that is at least 95% identical to the amino acid sequence of one of SEQ ID NO: 8.

40.-46. (canceled)

47. The chimeric protein of claim 1, wherein the chimeric protein has an amino acid sequence that is at least 95% identical to the amino acid sequence of one of SEQ ID NO: 9, SEQ ID NO: 10 or SEQ ID NO: 11.

48. A nucleic acid encoding the chimeric protein of claim 1.

49. A host cell, comprising the expression vector comprising the nucleic acid of claim 48.

50. A pharmaceutical composition, comprising the chimeric protein of claim 1.

51. A method of treating a cancer, a viral infection, or an inflammatory disease, comprising administering a pharmaceutical composition of claim 50 to a subject in need thereof.

52.-76. (canceled)
